SEQUENCE LISTING (100 sequences)

SEQ ID NO: 1 - protein, 14 aa, Artificial Sequence
  SARS-Cov2 spike protein Endogenous signal peptide
MFVFLVLLPLVSSQ

SEQ ID NO: 2 - protein, 23 aa, Artificial Sequence
  Tissue plasminogen activator signal peptide
MDAMKRGLCCVLLLCGAVFVSPS

SEQ ID NO: 3 - protein, 19 aa, Artificial Sequence
  Murine IgG HC signal peptide
MEWSWVFLFFLSVTTGVHS

SEQ ID NO: 4 - protein, 16 aa, Artificial Sequence
  Human IL2 signal peptide
MYRMQLLSCIALSLAL

SEQ ID NO: 5 - protein, 17 aa, Artificial Sequence
  Angiotensin-converting enzyme 2 signal peptide
MSSSSWLLLSLVAVTAA

SEQ ID NO: 6 - protein, 4 aa, Artificial Sequence
  Furin cleavage site mutation
SSAS

SEQ ID NO: 7 - protein, 4 aa, Artificial Sequence
  Furin cleavage site
GAAS

SEQ ID NO: 8 - protein, 4 aa, Artificial Sequence
  Furin cleavage site mutation
RRAR

SEQ ID NO: 9 - protein, 6 aa, Artificial Sequence
  Thrombin cleavage site
LVPRGS

SEQ ID NO: 10 - protein, 6 aa, Artificial Sequence
  TEV cleavage site
ENLYFQ

SEQ ID NO: 11 - protein, 4 aa, Artificial Sequence
  Flexible linker
GGGS

SEQ ID NO: 12 - protein, 4 aa, Artificial Sequence
  Flexible linker
GGSG

SEQ ID NO: 13 - protein, 5 aa, Artificial Sequence
  Flexible linker
GGSGG

SEQ ID NO: 14 - protein, 8 aa, Artificial Sequence
  HRV-3C Protease Cleavage Site
LEVLFQGP

SEQ ID NO: 15 - protein, 4 aa, Artificial Sequence
  Kinked linker
GGGP

SEQ ID NO: 16 - protein, 6 aa, Artificial Sequence
  Histidine tag (6x)
HHHHHH

SEQ ID NO: 17 - protein, 8 aa, Artificial Sequence
  His tag (8x)
HHHHHHHH

SEQ ID NO: 18 - protein, 10 aa, Artificial Sequence
  His tag (10x)
HHHHHHHHHH

SEQ ID NO: 19 - protein, 30 aa, Artificial Sequence
  T4 fibritin foldon domain
GSGYIPEAPRDGQAYVRKDGEWVLLSTFLG

SEQ ID NO: 20 - protein, 27 aa, Artificial Sequence
  Short T4 fibritin foldon domain
GYIPEAPRDGQAYVRKDGEWVLLSTFL

SEQ ID NO: 21 - protein, 104 aa, Artificial Sequence
  Clathrin trimerization domain
WKQSVELAKKDSLYKDAMQYASESKDTELAEELLQWFLQEEKRECFGACLFTCYDLLRPDVVLELAWRHNIMDFAMPYFIQVMKEYLTKVDKLDASESLRKEEE

SEQ ID NO: 22 - protein, 56 aa, Artificial Sequence
  Human Collagen Trimerization Domain
ASSGVRLWATRQAMLGQVHEVPEGWLIFVAEQEELYVRVQNGFRKVQLEARTPLPR

SEQ ID NO: 23 - protein, 33 aa, Artificial Sequence
  GCN4 based isoleucine zipper
RMKQLEDKVEELLSKNYHLENEVARLKKLVGER

SEQ ID NO: 24 - protein, 26 aa, Artificial Sequence
  Tetranectin trimerization domain
LKSRLDTLSQEVALLKEQQALQTVCL

SEQ ID NO: 25 - protein, 40 aa, Artificial Sequence
  p53 tetramerization domain
KPLDGEYFTLQIRGRERFEMFRELNEALELKDAQAGKEPG

SEQ ID NO: 26 - protein, 214 aa, Artificial Sequence
  Murine IgG1-Fc
VPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 27 - protein, 227 aa, Artificial Sequence
  Murine IgG1-Fc Dimerization domain
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 28 - protein, 330 aa, Artificial Sequence [sequence omitted]

SEQ ID NO: 29 - protein, 38 aa, Artificial Sequence
  Streptavidin binding peptide
MDEKTTGWRGGHVVEGLAGELEQLRARLEHHPQGQREP

SEQ ID NO: 30 - protein, 8 aa, Artificial Sequence
  Strep TagII
WSHPQFEK

SEQ ID NO: 31 - protein, 28 aa, Artificial Sequence
  TwinStrepTag
WSHPQFEKGGGSGGGSGGSAWSHPQFEK

SEQ ID NO: 32 - protein, 1213 aa, Artificial Sequence
  Full length Spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 33 - protein, 673 aa, Artificial Sequence
  S1
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSSAS

SEQ ID NO: 34 - protein, 223 aa, Artificial Sequence
  RBD
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 35 - protein, 528 aa, Artificial Sequence
  S2
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 36 - protein, 214 aa, Artificial Sequence
  Murine IgG Fc
VPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGI

SEQ ID NO: 37 - protein, 1256 aa, Artificial Sequence
  pxENB9-FLS-P.1
MFVFLVLLPLVSSQCVNFTTRNQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSGYIPEAPRDGQAYVRKDGEWVLLSTFLGGGSHHHHHHHH

SEQ ID NO: 38 - protein, 615 aa, Artificial Sequence
  ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 39 - protein, 182 aa, Artificial Sequence
  SARS-CoV2 nucleocapsid N-terminal domain
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQA

SEQ ID NO: 40 - protein, 1251 aa, Artificial Sequence
  pxAM-FLSA
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPLVPRGSGSGYIPEAPRDGQAYVRKDGEWVLLSTFLGHHHHHH

SEQ ID NO: 41 - protein, 1255 aa, Artificial Sequence
  pxAM-FLSSSAS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPLVPRGSGSGYIPEAPRDGQAYVRKDGEWVLLSTFLGHHHHHH

SEQ ID NO: 42 - protein, 1261 aa, Artificial Sequence
  pxENB1 - FLS
MFVFLVLLPLVSSQHHHHHHHHGGGSENLYFQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSLKSRLDTLSQEVALLKEQQALQTVCL

SEQ ID NO: 43 - protein, 1262 aa, Artificial Sequence
  pxENB2 - FLS
MFVFLVLLPLVSSQHHHHHHHHGGGSENLYFQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSGYIPEAPRDGQAYVRKDGEWVLLSTFL

SEQ ID NO: 44 - protein, 1231 aa, Artificial Sequence
  pxENB5 - FLS
MFVFLVLLPLVSSQHHHHHHHHGGGSENLYFQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 45 - protein, 1255 aa, Artificial Sequence
  pxENB8 - FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSLKSRLDTLSQEVALLKEQQALQTVCLGGGSHHHHHHHH

SEQ ID NO: 46 - protein, 1256 aa, Artificial Sequence
  PxENB9 - FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSGYIPEAPRDGQAYVRKDGEWVLLSTFLGGGSHHHHHHHH

SEQ ID NO: 47 - protein, 1225 aa, Artificial Sequence
  PxENB12 - FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSHHHHHHHH

SEQ ID NO: 48 - protein, 1264 aa, Artificial Sequence
  PxENBCP14-FLS
MDAMKRGLCCVLLLCGAVFVSPSVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSGYIPEAPRDGQAYVRKDGEWVLLSTFLGGGSHHHHHHHH

SEQ ID NO: 49 - protein, 1260 aa, Artificial Sequence
  PxENBCP15-FLS
MEWSWVFLFFLSVTTGVHSVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSGYIPEAPRDGQAYVRKDGEWVLLSTFLGGGSHHHHHHHH

SEQ ID NO: 50 - protein, 1257 aa, Artificial Sequence
  PxENBCP16-FLS
MYRMQLLSCIALSLALVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSGYIPEAPRDGQAYVRKDGEWVLLSTFLGGGSHHHHHHHH

SEQ ID NO: 51 - protein, 1298 aa, Artificial Sequence
  PxENBCP17-FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSGYIPEAPRDGQAYVRKDGEWVLLSTFLGGGSHHHHHHHHGGSGMDEKTTGWRGGHVVEGLAGELEQLRARLEHHPQGQREP

SEQ ID NO: 52 - protein, 1244 aa, Artificial Sequence
  PxENBCP18-FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSGYIPEAPRDGQAYVRKDGEWVLLSTFL

SEQ ID NO: 53 - protein, 1268 aa, Artificial Sequence
  PxENBCP19-FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSGYIPEAPRDGQAYVRKDGEWVLLSTFLGGGSHHHHHHHHGGSGWSHPQFEK

SEQ ID NO: 54 - protein, 1288 aa, Artificial Sequence
  PxENBCP20-FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSGYIPEAPRDGQAYVRKDGEWVLLSTFLGGGSHHHHHHHHGGSGWSHPQFEKGGGSGGGSGGSAWSHPQFEK

SEQ ID NO: 55 - protein, 1423 aa, Artificial Sequence
  PxENBEP17-FLS
MDAMKRGLCCVLLLCGAVFVSPSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQAGGSGGCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSHHHHHHHHHH

SEQ ID NO: 56 - protein, 1414 aa, Artificial Sequence
  PxENBEP18-FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGSGGMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQAGGGSHHHHHHHHHH

SEQ ID NO: 57 - protein, 1330 aa, Artificial Sequence
  PxENBEP21-FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSWKQSVELAKKDSLYKDAMQYASESKDTELAEELLQWFLQEEKRECFGACLFTCYDLLRPDVVLELAWRHNIMDFAMPYFIQVMKEYLTKVDKLDASESLRKGGGSHHHHHHHH

SEQ ID NO: 58 - protein, 1262 aa, Artificial Sequence
  PxENBEP22-FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSRMKQLEDKVEELLSKNYHLENEVARLKKLVGERGGGSHHHHHHHH

SEQ ID NO: 59 - protein, 1285 aa, Artificial Sequence
  PxENBEP23-FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSASSGVRLWATRQAMLGQVHEVPEGWLIFVAEQEELYVRVQNGFRKVQLEARTPLPRGGGSHHHHHHHH

SEQ ID NO: 60 - protein, 1372 aa, Artificial Sequence
  pxENBCP24 -FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSWKQSVELAKKDSLYKDAMQYASESKDTELAEELLQWFLQEEKRECFGACLFTCYDLLRPDVVLELAWRHNIMDFAMPYFIQVMKEYLTKVDKLDASESLRKGGGSHHHHHHHHGGSGMDEKTTGWRGGHVVEGLAGELEQLRARLEHHPQGQREP

SEQ ID NO: 61 - protein, 1304 aa, Artificial Sequence
  pxENBCP25 -FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSRMKQLEDKVEELLSKNYHLENEVARLKKLVGERGGGSHHHHHHHHGGSGMDEKTTGWRGGHVVEGLAGELEQLRARLEHHPQGQREP

SEQ ID NO: 62 - protein, 1327 aa, Artificial Sequence
  pxENBCP26 -FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSASSGVRLWATRQAMLGQVHEVPEGWLIFVAEQEELYVRVQNGFRKVQLEARTPLPRGGGSHHHHHHHHGGSGMDEKTTGWRGGHVVEGLAGELEQLRARLEHHPQGQREP

SEQ ID NO: 63 - protein, 687 aa, Artificial Sequence
  pO1-S1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPHHHHHH

SEQ ID NO: 64 - protein, 688 aa, Artificial Sequence
  pO2-S1
MYRMQLLSCIALSLALVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPHHHHHH

SEQ ID NO: 65 - protein, 699 aa, Artificial Sequence
  pO3-S1
MDAMKRGLCCVLLLCGAVFVSPSVTNSVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPHHHHHH

SEQ ID NO: 66 - protein, 695 aa, Artificial Sequence
  pO4-S1
MEWSWVFLFFLSVTTGVHSVTNSVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPHHHHHH

SEQ ID NO: 67 - protein, 698 aa, Artificial Sequence
  PxEBNCP1-S1
MFVFLVLLPLVSSQHHHHHHHHGGGSENLYFQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNS

SEQ ID NO: 68 - protein, 707 aa, Artificial Sequence
  PxEBNCP2-S1
MDAMKRGLCCVLLLCGAVFVSPSHHHHHHHHGGGSENLYFQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNS

SEQ ID NO: 69 - protein, 693 aa, Artificial Sequence
  PxEBNCP3-S1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGGSHHHHHHHH

SEQ ID NO: 70 - protein, 702 aa, Artificial Sequence
  PxEBNCP4-S1
MDAMKRGLCCVLLLCGAVFVSPSCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGGSHHHHHHHH

SEQ ID NO: 71 - protein, 908 aa, Artificial Sequence
  PxEBNCP5-S1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGI

SEQ ID NO: 72 - protein, 899 aa, Artificial Sequence
  PxEBNCP7-S1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGGPVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGI

SEQ ID NO: 73 - protein, 559 aa, Artificial Sequence
  PxEBNCP11-S2
MFVFLVLLPLVSSQHHHHHHHHGGGSENLYFQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKW

SEQ ID NO: 74 - protein, 554 aa, Artificial Sequence
  PxEBNCP13-S2
MFVFLVLLPLVSSQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSHHHHHHHH

SEQ ID NO: 75 - protein, 629 aa, Artificial Sequence
  PxWP-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADLEVLFQGPHHHHHH

SEQ ID NO: 76 - protein, 633 aa, Artificial Sequence
  PxEBN2-ACE2
MSSSSWLLLSLVAVTAAQHHHHHHHHGGGSENLYFQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 77 - protein, 627 aa, Artificial Sequence
  PxEBN4-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGSHHHHHHHH

SEQ ID NO: 78 - protein, 951 aa, Artificial Sequence
  PxEBN6-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADLVPRGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 665 aa, Artificial Sequence
  PxEBN9-ACE2b
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGSMDEKTTGWRGGHVVEGLAGELEQLRARLEHHPQGQREPHHHHHHHH

SEQ ID NO: 80 - protein, 614 aa, Artificial Sequence
  PxEBNCP9-ACE2
MYRMQLLSCIALSLALQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 81 - protein, 621 aa, Artificial Sequence
  PxEBNCP10-ACE2
MDAMKRGLCCVLLLCGAVFVSPSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 82 - protein, 657 aa, Artificial Sequence
  PxEBNCP13-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGSGMDEKTTGWRGGHVVEGLAGELEQLRARLEHHPQGQREP

SEQ ID NO: 83 - protein, 647 aa, Artificial Sequence
  PxEBNCP14-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGSGWSHPQFEKGGGSGGGSGGSAWSHPQFEK

SEQ ID NO: 84 - protein, 627 aa, Artificial Sequence
  PxEBNCP15-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGSGWSHPQFEK

SEQ ID NO: 85 - protein, 659 aa, Artificial Sequence
  PxEBNCP18-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGSKPLDGEYFTLQIRGRERFEMFRELNEALELKDAQAGKEPG

SEQ ID NO: 86 - protein, 617 aa, Artificial Sequence
  PxEBNCP19-ACE2
MEWSWVFLFFLSVTTGVHSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 87 - protein, 884 aa, Artificial Sequence
  PxEBNCP20-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGKGGSGMDEKTTGWRGGHVVEGLAGELEQLRARLEHHPQGQREP

SEQ ID NO: 88 - protein, 874 aa, Artificial Sequence
  PxEBNCP21-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGKGGSGWSHPQFEKGGGSGGGSGGSAWSHPQFEK

SEQ ID NO: 89 - protein, 842 aa, Artificial Sequence
  PxEBNCP22-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 90 - protein, 829 aa, Artificial Sequence
  PxEBNCP23-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 91 - protein, 701 aa, Artificial Sequence
  PxEBNCP24-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGSKPLDGEYFTLQIRGRERFEMFRELNEALELKDAQAGKEPGGGSGMDEKTTGWRGGHVVEGLAGELEQLRARLEHHPQGQREP

SEQ ID NO: 92 - protein, 615 aa, Artificial Sequence
  PxEBNCP25-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 93 - protein, 422 aa, Artificial Sequence
  PxEBNCP26-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIG

SEQ ID NO: 94 - protein, 511 aa, Artificial Sequence
  PxEBNCP27-ACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDAPAECLLLEPGLNEIMANSLDYNERLWAWESWRSEVGKALRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYS

SEQ ID NO: 95 - protein, 1333 aa, Artificial Sequence
  pxENBCP21-FLS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSWKQSVELAKKDSLYKDAMQYASESKDTELAEELLQWFLQEEKRECFGACLFTCYDLLRPDVVLELAWRHNIMDFAMPYFIQVMKEYLTKVDKLDASESLRKEEEGGGSHHHHHHHH

SEQ ID NO: 96 - protein, 1262 aa, Artificial Sequence; identical to SEQ ID NO: 58
  pxENBCP22-FLS

SEQ ID NO: 97 - protein, 1285 aa, Artificial Sequence; identical to SEQ ID NO: 59
  pxENBCP23-FLS

SEQ ID NO: 98 - protein, 1253 aa, Artificial Sequence
  pxENB9-FLS-B1.351
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHGAASSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSGYIPEAPRDGQAYVRKDGEWVLLSTFLGGGSHHHHHHHH

SEQ ID NO: 99 - protein, 1254 aa, Artificial Sequence
  pxENB9-FLS-B1.617.2
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSGYIPEAPRDGQAYVRKDGEWVLLSTFLGGGSHHHHHHHH

SEQ ID NO: 100 - protein, 1256 aa, Artificial Sequence
  pxENB9-FLS-B1.4.27
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGAASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGSGYIPEAPRDGQAYVRKDGEWVLLSTFLGGGSHHHHHHHH

(19) United States
(12) Patent Application Publication
(10) Pub. No.: US 2024/0400618 A1
(43) Pub. Date: Dec. 5, 2024

(54) FUSION PROTEINS COMPRISING SARS-COV-2 SPIKE PROTEIN OR THE RECEPTOR THEREOF

(71) Applicant: GRIFOLS DIAGNOSTIC SOLUTIONS INC., Emeryville, CA (US)

(72) Inventors: ELIZABETH A. BOOTH, Emeryville, CA (US); ELISABETE NASCIMENTO, Emeryville, CA (US); EFRAIN CEH PAVIA, Emeryville, CA (US); CHARLES HOLZ, Emeryville, CA (US); ZEBULON LAPOINT, Emeryville, CA (US); TRISTAN WASLEY, Emeryville, CA (US); COLLEN BROWN, Emeryville, CA (US); JODY MELTON WITT, Emeryville, CA (US)

(21) Appl. No.: 18/041,205

(22) PCT Filed: Aug. 17, 2021
(86) PCT No.: PCT/IB2021/057550

§ 371 Date: Sep. 28, 2023

Related U.S. Application Data
(60) Provisional application No. 63/066,678, filed on Aug. 17, 2020.

(51) Int. Cl. C07K 14/005; C12N 9/48

(52) U.S. Cl. CPC C07K 14/005; C12N 9/485; C12Y 304/17023; C12N 2770/20022; C12N 2770/20052

(57) ABSTRACT

A fusion protein containing the full length SARS-CoV-2 spike protein, or the S1 domain or the S2 domain of the SARS-CoV-2 spike protein or a fragment, or the human Angiotensin Converting Enzyme 2 (ACE2) receptor of the SARS-CoV-2 spike protein or a fragment, and a N-terminal signal peptide, and at least one of the following: a polyhistidine tag, a streptavidin binding domain, a linker, or an oligomerization tag.

### PRIORITY AND CROSS REFERENCE TO RELATED APPLICATIONS

This application is the U.S. National Phase application under 35 U.S.C. § 371 of International Application No. PCT/IB2021/057550, filed Aug. 17, 2021, designating in the U.S. and published as WO 2022/038504 A1 on Feb. 24, 2022, which claims the benefit of Provisional Application No. 63/066,678, filed Aug. 17, 2020. Any and all applications for which a foreign or a domestic priority is claimed is/are identified in the Application Data Sheet filed herewith and is/are hereby incorporated by reference in their entireties under 37 C.F.R. § 1.57.

### SEQUENCE LISTING

This application is being filed along with a Sequence Listing in electronic format. The replacement Sequence Listing is provided as a file entitled HOFMO28002APCCORRECTSEQLISTING.txt, which is 573,137 bytes in size and was created on Aug. 17, 2023. The information in the electronic format of the Sequence Listing is incorporated herein by reference in its entirety.

### FIELD

This application relates to the medical field of COVID-19 diagnosis or treatment, and in particular, it relates to fusion proteins comprising severe acute respiratory syndrome coronavirus 2 (SARS-CoV-2) spike protein or one of the subunits 1 or 2 of SARS-CoV-2 spike protein or the human Angiotensin Converting Enzyme 2 (ACE2) receptor of the SARS-CoV-2 spike protein. Said fusion proteins are useful for the development of assays capable of screening reagents that inhibit binding of the viral spike protein to human ACE2.

### BACKGROUND

SARS-CoV-2 is an enveloped RNA virus from the Coronaviridae family (Gorbalenya, A. E, et al., 2020, Nature Microbiology, 5(4):p. 536-544) that has several structural components, including Spike (S), Envelope (E), Membrane (M) and Nucleocapsid (N) proteins (Lu, R., et al., 2020, Lancet 395(10224):p. 565-574). The S protein consists of two subunits (S1 and S2) that form a trimer on the viral membrane; S1 contains the RBD which is responsible for binding to the ACE2 host cell receptor (Hoffmann, M., et. al., 2020, Cell, 181(2):p. 271-280.e8), while S2 enables the fusion between the host and viral membranes (Lan, J., et al., 2020, Nature, 581(7807):215-220; Wrapp, D., et al., 2020, Science, 367(6483):p. 1260-1263).

SARS-CoV-2 has caused a widespread COVID-19 pandemic that infected millions worldwide and claimed hundreds of thousands of lives. Currently, the main and most accurate method of diagnosis is by PCR testing of nasopharyngeal swabs (Peng et al., 2020, J Med Virol. 24; 10.1002/jmv.25936); yet, there is an urgent need to develop reliable, highly sensitive and specific antibody tests capable of identifying all infected individuals, irrespective of clinical symptoms. This information will be critical to establish community surveillance and implement policies that contain the viral spread.

The US Food and Drug Administration (FDA) has granted Emergency Use Authorizations (EUA) to multiple immunoassay tests in the market, but none of those assays has been fully validated. Because of the lack of validated immunoassays, key to understand risk, epidemiological factors, pathogenesis and mortality, we developed recombinant Spike and ACE2 molecular designs aimed at being a reagent in SARS-CoV-2 immunoassays.

Although there are multiple ways to neutralize the virus, due to its dependence on ACE2 for entry, there is still the need for providing composition that can be useful to neutralize the virus and also that can be useful to detect neutralizing reagents that interrupt the binding of the spike protein with ACE2.

The inventors of the present invention have developed fusion proteins comprising the full length SARS-CoV-2 spike protein (FLS), or the S1 domain or the S2 domain of the SARS-CoV-2 spike protein, or the human Angiotensin Converting Enzyme 2 (ACE2) receptor of the SARS-CoV-2 spike protein, with other structural elements or domains that can be used for the development of assays capable of screening reagents that inhibit binding of the viral spike protein to human ACE2. Although there are multiple ways to neutralize the virus, due to its dependence on ACE2 for entry, this represents one assay that could detect neutralizing reagents that interrupt the binding of the Spike protein with ACE2. The novel recombinant fusion proteins of the present invention have improved affinity, expression, solubility, manufacturability, and assay sensitivity.

### SUMMARY

In a first aspect, the present invention refers to a fusion protein comprising the full length SARS-CoV-2 spike protein, or the S1 domain or the S2 domain of the SARS-CoV-2 spike protein or a fragment thereof, or the human Angiotensin Converting Enzyme 2 (ACE2) receptor of the SARS-CoV-2 spike protein or a fragment thereof, and a N-terminal signal peptide, and at least one of a polyhistidine tag, a streptavidin binding domain, a linker, or an oligomerization tag.

In one embodiment, said N-terminal signal peptide is selected from a spike endogenous signal peptide, a tissue plasminogen activator (tPa) signal peptide, a human interleukin 2 (hIL2) signal peptide, a murine immunoglobulin G (IgG) heavy chain signal peptide and a angiotensin-converting enzyme 2 (ACE2) signal peptide.

In one embodiment, the amino acid sequence of said N-terminal signal peptide is selected from SEQ ID NO: 1, SEQ ID NO: 2, SEQ ID NO: 3, SEQ ID NO: 4 or SEQ ID NO: 5.

In another embodiment, the amino acid sequence of said linker is a flexible linker or a kinked linker. In another embodiment said linker has an amino acid sequence selected from SEQ ID NO: 11, SEQ ID NO: 12, SEQ ID NO: 13, or SEQ ID NO: 15.

In another embodiment, said polyhistidine tag consists of 4, 6, 8, 10, or 12 histidine residues. In another embodiment, the amino acid sequence of said polyhistidine tag is selected from SEQ ID NO: 16, SEQ ID NO: 17 or SEQ ID NO: 18.

In another embodiment, said oligomerization tag is selected from a T4 fibritin foldon domain, a short T4 fibritin foldon domain, a clathrin trimerization domain, a human collagen trimerization domain, a CGN4 trimerization domain, a tetranectin trimerization domain, a p53 tetramerization domain and a murine or human Fc domain. In another embodiment, the amino acid sequence of said oligomerization tag is selected from SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28 or SEQ ID NO: 36.

In another embodiment, the amino acid sequence of said streptavidin binding domain is selected from SEQ ID NO: 29, SEQ ID NO: 30, or SEQ ID NO: 31.

In some embodiments of the fusion protein of the present invention, the amino acid sequence of said full length SARS-CoV-2 spike protein is set forth in SEQ ID NO: 32.

In some embodiments of the fusion protein of the present invention, the amino acid sequence of said S1 domain of the SARS-CoV-2 spike protein or a fragment thereof is set forth in SEQ ID NO: 33.

In some embodiments of the fusion protein of the present invention, the amino acid sequence of said S2 domain of the SARS-CoV-2 spike protein or a fragment thereof is set forth in SEQ ID NO: 35.

In some embodiments of the fusion protein of the present invention, the amino acid sequence of said human Angiotensin Converting Enzyme 2 (ACE2) receptor of the SARS-CoV-2 spike protein or a fragment thereof is set forth in SEQ ID NO: 38.

In some embodiments of the fusion protein of the present invention, the full length SARS-CoV-2 spike protein, or the S1 domain or the S2 domain of the SARS-CoV-2 spike protein or a fragment thereof, comprises a mutation in one or more of the following positions: N354, D364, V367, G404, R408, A435, W436, G467, A475, T478, N481, G485, F490, Q493, G496, Q498, N501, G502, V503 or D614, relative to SEQ ID NO: 32.

In some embodiments of the fusion protein of the present invention, the ACE2 protein or the fragment thereof comprises three alanine mutations which are Q139A, Q175A and N137A, relative to SEQ ID NO: 38.

In some embodiments, the fusion protein of the present invention comprises the stabilizing mutations K986P and V987P, relative to SEQ ID NO: 32.

In another embodiment, the fusion protein of the present invention does not contain a site recognized by the furin. In another embodiment, the fusion protein of the present invention contains a thrombin cleavage site or a TEV cleavage site or a HRV-3C protease cleavage site.

In some embodiments, the fusion proteins of the present invention comprise the amino acid sequence selected from SEQ ID NO: 37, or SEQ ID NO: 40 to SEQ ID NO: 100.

In some embodiments, the fusion proteins of the present invention, when comprising the full length the SARS-CoV-2 spike protein, comprises the amino acid sequence selected from SEQ ID NO: 37 or SEQ ID NO: 40 to SEQ ID NO: 62 or SEQ ID NO: 95 to SEQ ID NO: 100.

In other embodiments, the fusion proteins of the present invention, when comprising the S1 domain of the SARS-CoV-2 spike protein or the fragment thereof, comprises the amino acid sequence selected from SEQ ID NO: 63 to 72.

In other embodiments, the fusion proteins of the present invention, when comprising the S2 domain of the SARS-CoV-2 spike protein or the fragment thereof, comprises the amino acid sequence selected from SEQ ID NO: 73 to 74.

In other embodiments, the fusion proteins of the present invention, when comprising the human Angiotensin Converting Enzyme 2 (ACE2) receptor of the SARS-CoV-2 spike protein or the fragment thereof, comprises the amino acid sequence selected from SEQ ID NO: 75 to 94.

In another aspect, the present invention refers to a cell, comprising the fusion protein as described above.

In a further aspect, the present invention refers to a nucleotide sequence encoding the fusion protein as described above, a promoter operably linked to the nucleotide sequence and a selectable marker.

In another aspect, the present invention refers to a cell comprising the above-mentioned nucleic acid.

Finally, the present invention refers to a composition comprising the above-mentioned fusion protein, and a solid support, wherein the fusion protein covalently or non-covalently bound to the solid support.

### BRIEF DESCRIPTION OF THE DRAWINGS

FIG. 1. SEC-MALS data for 50 μg injections of three ACE2 fusion proteins pxEBN4-ACE2 (red), pxEBN9-ACE2 (green), and pxWP-ACE2 (blue). The A280 trace peaks are intersected by the measured molar mass of each peak and the flat molar mass data across each peak is indicative of a monodisperse sample. pxEBN4-ACE2: MW 81.1±1.9% kDa, Mw/Mn 1.001±2.67%; pxEBN9-ACE2: 83.4±2.2%, Mw/Mn 1.001±3.12%; pxWP-ACE2: 80.9 kDa±3.1%, Mw/Mn 1.001±4.33%.

FIG. 2. BLI analysis of binding between GDS ACE2 with commercial RBD and GDS ACE2 with literature FLS (pxAM-FLS-SSAS). A) Binding affinity (KD) was measured for commercial RBD with Biotinylated pxEBN4-ACE2 loaded on Streptavidin tips. A 2× serial dilution of RBD from 100 nM to 3.13 nM was used with a reported binding affinity of 0.20±0.004 nM. B) Binding affinity (KD) was measured for pxAM-FLS-SSAS with Biotinylated pxEBN4-ACE2 loaded on Streptavidin tips. A 2× serial dilution of trimeric FLS from 33 nM to 1.04 nM was used with a reported binding affinity of 0.13±0.001 nM.

FIG. 3. BLI analysis of binding between biotinylated GDS pxEBN4-ACE2 and six candidate FLS proteins. FLS proteins included (A) pxENB1-His-FLS (B) pxENB2-FLS (C) pxENB9-FLS (D) pxENB8-FLS (E) pxENB5FLS (F) pxENB12-FLS prepared as a 2× serial dilution from 33.3 nM to 1.04 nM. KD values are reported in Table 6.

FIG. 4. Rabbit pAbs raised against S1 antigen are capable of inhibiting the binding of FLS with ACE2.

FIG. 5. Biolayer interferometry sensorgrams illustrating human ACE2 receptor-Streptavidin tip loading and binding RBD: Comparison of binding curves of immobilized ACE2 (gray) and ACE2-SBP (blue) loaded on streptavidin biosensors at 35.85 μg/mL and associating with recombinant SARS-CoV-2 RBD (pxENB17-RBD) at a concentration of 6.25 nM.

FIG. 6. pxEBN4-ACE2-AAA-His biotinylated at a 2:1 molar ratio (biotin:protein; assuming monomerization of ACE2 construct) and loaded at a concentration of 35.86 μg/mL onto SA Octet Tips. All five FLS variants were separately associated to bt-ACE2, in a two-fold serial dilution from 500 nM (assuming trimerization of FLS construct). For analysis, a minimum of 4 curves is necessary to provide reliable KD data.

FIG. 7. pxEBN4-ACE2-AAA-His biotinylated at a 2:1 molar ratio (biotin:protein; assuming monomerization of ACE2 construct) and loaded at a concentration of 35.86 μg/mL onto SA Octet Tips. All FLS/S1/S2/RBD constructs were separately associated to bt-ACE2, at 100 nM and 33.33 nM (accounting for dimerization and trimerization domains). A. Initial FLS Constructs. B. SARS-CoV-2 S1 Constructs. C. SARS-CoV-2 FLS Constructs with Alternative Signal Peptides. D. SARS-CoV-2 Nuclear Capsid—Spike Fusions. E. SARS-CoV-2 RBD Constructs.

FIG. 8. FLS constructs loaded at 33.3 nM (assuming trimerization) onto SA Octet Tips. ACE2 was associated to FLS constructs at. 33 nM (accounting for dimerization). Preliminary KD and response data can be found in Table 4 is used to compare constructs.

FIG. 9. Recombinant RBD was biotinylated at a 1:1 molar ratio (biotin:protein) and loaded at a concentration of 1.68 μg/mL onto SA Octet Tips. All ACE2 constructs were separately associated to bt-ACE2, at 100 nM and 33.33 nM (accounting for multimerization domains). A. ACE2 Constructs with p53 Subunit fusions. B. ACE2 Constructs with mIgG Fc Fusions. C. ACE2 Constructs with Alternative Signal Peptides. D. ACE2 Native Constructs with AAA mutation.

FIG. 10. ACE2 constructs loaded at a concentration of 33.3 nM by their respective Streptavidin tags onto SA Octet Tips. pxENB9-FLS-CT-His was associated to the ACE2 constructs at 33.33 nM (accounting for multimerization domains).

### DETAILED DESCRIPTION

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used and will be apparent to those of skill in the art. All publications and other references mentioned herein are incorporated by reference in their entirety. In case of conflict, the present specification, including definitions, will control. The materials, methods, and examples are illustrative only and not intended to be limiting.

Each embodiment in this specification is to be applied mutatis mutandis to every other embodiment unless expressly stated otherwise.

The following terms, unless otherwise indicated, shall be understood to have the following meanings:

As used herein, the term “nucleic acid” refers to any materials comprised of DNA or RNA. Nucleic acids can be made synthetically or by living cells.

As used herein, the term “protein” or refers to large biological molecules, or macromolecules, consisting of one or more chains of amino acid residues. Many proteins are enzymes that catalyze biochemical reactions and are vital to metabolism. Proteins also have structural or mechanical functions, such as actin and myosin in muscle and the proteins in the cytoskeleton, which form a system of scaffolding that maintains cell shape. Other proteins are important in cell signaling, immune responses, cell adhesion, and the cell cycle. However, proteins may be completely artificial or recombinant, i.e., not existing naturally in a biological system.

As used herein, the term “polypeptide” refers to both naturally-occurring and non-naturally-occurring proteins, and fragments, mutants, derivatives and analogs thereof. A polypeptide may be monomeric or polymeric. A polypeptide may comprise a number of different domains (peptides) each of which has one or more distinct activities.

As used herein, the term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, the term “fusion protein” refers to proteins comprising two or more amino acid sequences that do not co-exist in naturally-occurring proteins. A fusion protein may comprise two or more amino acid sequences from the same or from different organisms. The two or more amino acid sequences of a fusion protein are typically in frame without stop codons between them and are typically translated from mRNA as part of the fusion protein.

The term “fusion protein” and the term “recombinant” can be used interchangeably herein.

As used herein, the term “antigen” refers to a biomolecule that binds specifically to the respective antibody. An antibody from the diverse repertoire binds a specific antigenic structure by means of its variable region interaction.

The terms “antibody” or “immunoglobulin”, as used herein, have the same meaning, and will be used equally in the present invention. The term “antibody” as used herein refers to immunoglobulin molecules and immunologically active portions of immunoglobulin molecules, i.e., molecules that contain an antigen binding site that specifically binds an antigen. As such, the term antibody encompasses not only whole antibody molecules, but also antibody fragments or derivatives.

The term “binding affinity”, as used herein, refers to the strength of interaction between an antigen's epitope and an antibody's antigen binding site.

As used herein, a “promoter” is a specific nucleic acid sequence that is recognized by a DNA-dependent RNA polymerase (“transcriptase”) as a signal to bind to the nucleic acid and begin the transcription of RNA at a specific site.

The terms “modified sequence” and “modified genes” are used interchangeably herein to refer to a sequence that includes a deletion, insertion or interruption of naturally occurring nucleic acid sequence. In some preferred embodiments, the expression product of the modified sequence is a truncated protein (e.g., if the modification is a deletion or interruption of the sequence). In some particularly preferred embodiments, the truncated protein retains biological activity. In alternative embodiments, the expression product of the modified sequence is an elongated protein (e.g., modifications comprising an insertion into the nucleic acid sequence). In some embodiments, an insertion leads to a truncated protein (e.g., when the insertion results in the formation of a stop codon). Thus, an insertion may result in either a truncated protein or an elongated protein as an expression product.

As used herein, the terms “mutant sequence” and “mutant gene” are used interchangeably and refer to a sequence that has an alteration in at least one codon occurring in a host cell's wild-type sequence. The expression product of the mutant sequence is a protein with an altered amino acid sequence relative to the wild-type. The expression product may have an altered functional capacity (e.g., enhanced binding affinity).

The term “fragment” as used herein, refers to a portion of an amino acid sequence wherein said portion is smaller than the entire amino acid sequence.

The term “receptor-binding domain” or “RBD” refers to a protein in SARS-CoV-2 S that bound strongly to human and bat angiotensin-converting enzyme 2 (ACE2) receptors.

The term “spike protein”, “S protein” or “S” refers to a large type I transmembrane protein ranging from 1,160 amino acids for avian infectious bronchitis virus (IBV) and up to 1,400 amino acids for feline coronavirus (FCoV). In addition, this protein is highly glycosylated as it contains 21 to 35 N-glycosylation sites. Spike proteins assemble into trimers on the virion surface to form the distinctive “corona”, or crown-like appearance. The ectodomain of all CoV spike proteins share the same organization in two domains: a N-terminal domain named S1 that is responsible for receptor binding and a C-terminal S2 domain responsible for fusion. CoV diversity is reflected in the variable spike proteins (S proteins), which have evolved into forms differing in their receptor interactions and their response to various environmental triggers of virus-cell membrane fusion. It's been reported that 2019-nCoV can infect the human respiratory epithelial cells through interaction with the human ACE2 receptor. Indeed, the recombinant Spike protein can bind with recombinant ACE2 protein.

The term “angiotensin converting enzyme 2” or “ACE2” refers to an enzyme attached to the cell membranes of cells in the lungs, arteries, heart, kidney, and intestines. ACE2 lowers blood pressure by catalysing the hydrolysis of angiotensin II (a vasoconstrictor peptide) into angiotensin (1-7) (a vasodilator). ACE2 counters the activity of the related angiotensin-converting enzyme (ACE) by reducing the amount of angiotensin-II and increasing Ang(1-7) making it a promising drug target for treating cardiovascular diseases. ACE2 also serves as the entry point into cells for some coronaviruses, including HCoV-NL63, SARS-CoV, and SARS-CoV-2. The human version of the enzyme is often referred to as hACE2. As used herein, the term “N-terminal signal peptide” is a short peptide (usually 10-30 amino acids long) present at the N-terminus of the majority of newly synthesized proteins that are destined toward the secretory pathway. These proteins include those that reside either inside certain organelles (the endoplasmic reticulum, Golgi or endosomes), secreted from the cell, or inserted into most cellular membranes. Although most type I membrane-bound proteins have signal peptides, the majority of type II and multi-spanning membrane-bound proteins are targeted to the secretory pathway by their first transmembrane domain, which biochemically resembles a signal sequence except that it is not cleaved. They are a kind of target peptide.

As used herein, the term “purification tag” or “affinity tag” refers to a polypeptide used to purify proteins that simplifies purification and enables use of standard protocols. In the present invention, the purification tag is polyhistidine tag of 4, 6, 7, 8, 9, 10, 11 or 12. Preferably, the histidine tag has 4, 6, 8, 10, or 12 histidine residues.

As used herein, the term “linker” refers to a polypeptide comprising of 1-10 amino acids, preferably 3-6 amino acids. The amino acids of the linker may be selected from the group consisting of leucine (Leu, L), isoleucine (Ile, I), alanine (Ala, A), glycine (Gly, G), valine (Val, V), proline (Pro, P), lysine (Lys, K), arginine (Arg, R), Serine (Ser, S), asparagine (Asn, N), and glutamine (Gln, Q), tryptophan (Trp, W), methionine (Met, M), aspartic acid (Asp, D), cysteine (Cys, C), glutamic acid (Glu, E), histidine (His, H), phenylalanine (Phe, F), threonine (The, T), and tyrosine (Tyr, Y). In some preferred embodiments, the linker is a flexible linker that may consist of a sequence of consecutive amino acids that typically include at least one glycine and at least one serine. Exemplary flexible linkers include the amino acid sequences set forth in SEQ ID NO: 11 to SEQ ID NO: 13, although the precise amino acid sequence of the linker is not particularly limiting. In another embodiment, said linker is a kinked linker, which contains at least one proline, which kink the amino acid chain. Exemplary kinked linker includes the amino acid sequences set forth in SEQ ID NO: 15.

As used herein, the term “oligomerization tag” refers to a polypeptide for increasing assay avidity and sensitivity. In the present invention, the oligomerization tag is selected from a T4 fibritin foldon domain, a short T4 fibritin foldon domain, a clathrin trimerization domain, a human collagen trimerization domain, a CGN4 trimerization domain, a tetranectin trimerization domain, a p53 tetramerization domain and a murine or human Fc domain, but other oligomerization tags can also be used.

The term “diagnostic” or “diagnosed”, as used herein, means identifying the presence or nature of a pathologic condition or a patient susceptible to a disease. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein, the term “Biolayer Interferometry (BLI)” is a label-free technology for measuring biomolecular interactions. It is an optical analytical technique that analyzes the interference pattern of white light reflected from two surfaces: a layer of immobilized protein on the biosensor tip, and an internal reference layer. Any change in the number of molecules bound to the biosensor tip causes a shift in the interference pattern that can be measured in real-time.

1. FUSION PROTEINS The present invention refers to a fusion protein comprising the full length SARS-CoV-2 spike protein, or the S1 domain or the S2 domain of the SARS-CoV-2 spike protein or a fragment thereof, or the human Angiotensin Converting Enzyme 2 (ACE2) receptor of the SARS-CoV-2 spike protein or a fragment thereof, and a N-terminal signal peptide, and at least one of a polyhistidine tag, a streptavidin binding domain, a linker, or an oligomerization tag.

The SARS-CoV-2 full length Spike (FLS, GenBank MN908947.3) comprises two domains, namely S1 and S2, which are responsible for the binding step. S1 domain is involved in host cell receptor recognition and binding. Once S1 domain binds the receptor, it results in a conformational change of the S2 domain which facilitates the fusion between the viral envelope and the plasma membrane of its target cell. S2 domain contains the putative fusion peptide as well as heptad repeat HR1 and HR2.

Antibody binding on the N-terminal of SARS-CoV-2 spike protein can inhibit its activity and blocks the infection as a result. Furthermore, spike protein is the target of CD4 and CD8 immune cells. For these reasons, SARS-CoV-2 spike protein is considered as a potential target for the neutralization of SARS-CoV-2 virus.

The amino acid sequence of the full length SARS-CoV-2 Spike protein is set forth in SEQ ID NO: 32. In some embodiments of the present invention, the fusion protein comprises the full length SARS-CoV-2 Spike protein having the amino acid sequence set forth in SEQ ID NO: 32 or an amino acid sequence of at least 90% sequence identity with SEQ ID NO: 32. In some preferred embodiments, said full length SARS-CoV-2 Spike protein has an amino acid sequence of at least 95%, or at least 97%, or at least 98% identity with SEQ ID NO: 32.

The amino acid sequence of the S1 domain of the SARS-CoV-2 spike protein is set forth in SEQ ID NO: 33. In some embodiments of the present invention, the fusion protein comprises the S1 domain of the SARS-CoV-2 spike protein or a fragment thereof having the amino acid sequence set forth in SEQ ID NO: 33 or an amino acid sequence of at least 90% sequence identity with SEQ ID NO: 33. In some preferred embodiments, said S1 domain of the SARS-CoV-2 spike protein or the fragment thereof has an amino acid sequence of at least 95%, or at least 97%, or at least 98% identity with SEQ ID NO: 33.

The amino acid sequence of the RBD domain of the SARS-CoV-2 spike protein is set forth in SEQ ID NO: 34. In some embodiments of the present invention, the fusion protein comprises the RBD domain of the SARS-CoV-2 spike protein having the amino acid sequence set forth in SEQ ID NO: 34 or an amino acid sequence of at least 90% sequence identity with SEQ ID NO: 34.

The amino acid sequence of the S2 domain of the SARS-CoV-2 spike protein is set forth in SEQ ID NO: 35. Said S2 domain natively includes a furin cleavage site (RRAR), which is recognized by furin that cleaves the protein in preparation for cell entry. In the present application said furin cleavage site has been mutated in order to remove the furin recognition site and produce a full length spike protein. The mutated furin cleavage sites are set forth in SEQ ID NO: 6, SEQ ID NO: 7 or SEQ ID NO: 8. Furin is commonly expressed and inclusion of the furin cleavage site could result in degradation of the protein during production and purification. Therefore, some of the fusion proteins of the present invention do not contain a site recognized by the furin.

In some embodiments of the present invention, the fusion protein comprises the S2 domain of the SARS-CoV-2 spike protein or a fragment thereof having the amino acid sequence set forth in SEQ ID NO: 35, or an amino acid sequence of at least 90% sequence identity with SEQ ID NO: 35. In some preferred embodiments, said S2 domain of the SARS-CoV-2 spike protein or the fragment thereof has an amino acid sequence of at least 95%, or at least 97%, or at least 98% identity with SEQ ID NO: 35.

The amino acid sequence of the ACE2 protein is set forth in SEQ ID NO: 38. In some embodiments of the present invention, the fusion protein comprises the human Angiotensin Converting Enzyme 2 (ACE2) receptor of the SARS-CoV-2 spike protein or a fragment having the amino acid sequence set forth in SEQ ID NO: 38 or an amino acid sequence of at least 90% sequence identity with SEQ ID NO: 38. In some preferred embodiments, said ACE2 protein or the fragment thereof has an amino acid sequence of at least 95%, or at least 97%, or at least 98% identity with SEQ ID NO: 38.

In some embodiments of the present invention, the fusion protein comprises a N-terminal signal peptide. Said N-terminal signal peptide could result in improved expression and/or secretion of the protein during recombinant production. Moreover, inclusion of different signal peptides can alter post translational modification (PTMs) and potentially the function of the protein. Therefore, it is non-obvious that the fusion proteins of the present invention can be produced or be functional.

In some preferred embodiments, said N-terminal signal peptide is selected from a spike endogenous signal peptide, a tissue plasminogen activator (tPa) signal peptide, a human interleukin 2 (hIL2) signal peptide, a murine immunoglobulin G heavy chain (mIgGHC) signal peptide and a angiotensin-converting enzyme 2 (ACE2) signal peptide. Inclusion of each of these signal peptides result in improved expression and/or secretion of the protein during mammalian production. Moreover, inclusion of different signal peptides can alter the post-translational modifications and potentially the function of the protein. In some preferred embodiments, the amino acid sequence of said N-terminal signal peptide is selected from SEQ ID NO: 1, SEQ ID NO: 2, SEQ ID NO: 3, SEQ ID NO: 4 and SEQ ID NO: 5. In other embodiments, said N-terminal signal peptide has an amino acid sequence of at least 95%, or at least 97%, or at least 98% identity with SEQ ID NO: 1, SEQ ID NO: 2, SEQ ID NO: 3, SEQ ID NO: 4 or SEQ ID NO: 5. Linkers can be also present in the fusion proteins of the present invention. In one embodiment, said linker can be a flexible linker or a kinked linker. Flexible linkers are included when fusing domains of different proteins together. These flexible linkers may help to improve the tolerance for assembly of those domains, and are often a combination of glycine and serine while proline can be added to kink the protein. However, it is not obvious to the skilled person if the inclusion of the selected linkers would produce functional fusion proteins. In one embodiment, said linker is a flexible linker and has an amino acid sequence selected from SEQ ID NO: 11, SEQ ID NO: 12, or SEQ ID NO: 13. In other embodiment, said linker is a kinked linker and has the amino acid sequence set forth in SEQ ID NO: 15.

The fusion proteins of the present invention can be obtained by methods well-known to the skilled person. For example, said fusion proteins can be obtained recombinantly in bacteria, yeasts, fungi, or mammalian cells. In one embodiment, the fusion proteins of the present invention are produced in prokaryotic cells, such as Escherichia coli, but other prokaryotic cells can be used. In another embodiment, the fusion proteins of the present inventions are produced in human embryotic kidney (HEK) or Chinese hamster ovary (CHO) cells, but other eukaryotic cells can be used.

The fusion proteins of the present invention can be purified from the cells by methods well known to the skilled person. Said methods include, without limitation, filtration, conjugation, affinity chromatography, ion exchange chromatography, hydrophobic interaction chromatography, and size exclusion chromatography.

As previously described, the use of polyhistidine tags simplifies purification and enables use of standard protocols in the production of fusion proteins. For example, the histidine (His) tag (also known as polyhistidine or polyHis) is known to be useful, for example, in the purification by Immobilized Metal Affinity Chromatography (IMAC). The present inventors have included N-terminal polyhistidine-TEV tags and C-terminal polyhistidine tags. These tags may have an effect in the production of the protein as well as its functionality and aggregation state. Said impact is not obvious to a skilled person. Other uses of the polyhistidine tag are also well-known by the skilled person and therefore, the polyhistidine tags of the present invention are not limited to the purification functionality. In the present invention, said polyhistidine tag consists of 4, 6, 8, 10, or 12 histidine residues, preferably said polyhistidine tag can be selected from SEQ ID NO: 16, SEQ ID NO: 17 or SEQ ID NO: 18.

Different streptavidin binding domains were used in the fusion proteins of the present invention. These domains aid plate coating or conjugation of fluorophores or HRP tags for readout. Using a streptavidin binding domain can aid plate coating by improving orientation of the protein on the plate and avoiding unintentional labeling of key residues for protein-protein binding. Similarly, avoiding labeling key residues for protein-protein binding is important for reporter protein functionality. In one embodiment, said streptavidin binding domain has the amino acid sequence selected from SEQ ID NO: 29, SEQ ID NO: 30 or SEQ ID NO: 31.

In some embodiments, the fusion protein of the present invention comprises an oligomerization tags or domains. In some preferred embodiments, said oligomerization tag is selected from a T4 fibritin foldon domain, a short T4 fibritin foldon trimerization domain, a clathrin trimerization domain, a human collagen trimerization domain, a CGN4 trimerization domain, a tetranectin trimerization domain, a p53 tetramerization domain and a murine or human Fc domain.

In some preferred embodiments, the murine Fc domain is a murine IgG1 domain comprising or not the hinge region. In some preferred embodiments, the human Fc domain is a human IgG1 domain comprising or not the hinge region.

In some preferred embodiments, the amino acid sequence of said oligomerization tag is selected from SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28, or SEQ ID NO: 36. In other embodiments, said oligomerization tag has an amino acid sequence of at least 95%, or at least 97%, or at least 98% identity with SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, or SEQ ID NO: 36.

Some oligomerization domains specific for ACE2 were included in order to create avidity effects between the full length spike trimer and ACE oligomers. For that, ACE2 was fused with the Fc fusions to generate dimers and C-terminal p53 domains to generate tetramers.

As previously explained, some of the fusion proteins of the present invention do not contain a site recognized by the furin. The Spike protein natively includes a RRAR site recognized by furin that cleavages the protein in preparation for cell entry. The present inventors has used the novel GAAS mutation to remove the furin recognition site and produce a full-length spike protein. Furin is commonly expressed by mammalian cells and inclusion of the furin cleavage site could result in degradation of the protein during production and purification. In other embodiments, the fusion proteins of the present invention may contain a Thrombin cleavage site or a TEV cleavage site or HRV-3C cleavage site. In one embodiment, said Thrombin cleavage site has the amino acid sequence of SEQ ID NO: 9. In another embodiment, said TEV cleavage site has the amino acid sequence of SEQ ID NO: 10. In a further embodiment, said HRV-3C cleavage site has the amino acid sequence of SEQ ID NO: 14.

### II. EXEMPLARY FUSION PROTEINS

The fusion protein of the present invention may include at least one point mutation. In some embodiments, said at least one point mutation is within the SARS-CoV-2 spike protein. In more preferred embodiments, the SARS-CoV-2 spike protein of the fusion proteins of the present invention comprises a mutation in one or more of the following positions relative to SEQ ID NO: 32: N354, D364, V367, G404, R408, A435, W436, G467, A475, T478, N481, G485, F490, Q493, G496, Q498, N501, G502, V503 or D614.

As the fusion proteins of the present invention may comprise the full length SARS-CoV-2 spike protein, or the S1 domain or the S2 domain of the SARS-CoV-2 spike protein or a fragment thereof, said point mutations may be comprised in any of said domains.

In other embodiments, the at least one point mutation is within ACE2 domain of the fusion proteins of the present invention. In some preferred embodiments said at least one mutation are at least one of three alanine mutations in relation to SEQ ID NO: 38: Q139A, Q175A and N137A. These mutations are found at the neck of the protein where a weak dimerization occurs, and were included to aid formation of ACE2 oligomers mitigating any potential aggregation issues.

In addition, two K986P and V987P mutations (relative to SEQ ID NO: 32) have been included in some of the fusion proteins of the present invention to aid protein stabilization (Kirchdoerfer, R. N. et al. Sci. Rep. 1-11 (2018)).

Exemplary fusion proteins of the present invention comprise the full length the SARS-CoV-2 spike protein. In some embodiments, said fusion proteins comprise the amino acid sequence selected from SEQ ID NO: 37 or 40 to 62 or 95 to 100, or an amino acid sequence of at least 90%, or at least 95% sequence identity with one selected from SEQ ID NO: 37, or 40 to 62 or 95 to 100.

Exemplary fusion proteins of the present invention comprise the S1 domain of the SARS-CoV-2 spike protein or the fragment thereof. In some embodiments, said fusion proteins comprise the amino acid sequence selected from SEQ ID NO: 63 to 72, or an amino acid sequence of at least 90%, or at least 95% sequence identity with one selected from SEQ ID NO: 63 to 72.

Exemplary fusion proteins of the present invention comprise the S2 domain of the SARS-CoV-2 spike protein or the fragment thereof. In some embodiments, said fusion proteins comprise the amino acid sequence selected from SEQ ID NO: 73 to 74, or an amino acid sequence of at least 90%, or at least 95% sequence identity with one selected from SEQ ID NO: 73 to 74.

Exemplary fusion proteins of the present invention comprise the human Angiotensin Converting Enzyme 2 (ACE2) receptor of the SARS-CoV-2 spike protein or the fragment thereof. In some embodiments, said fusion proteins comprise the amino acid sequence selected from SEQ ID NO: 75 to 94, or an amino acid sequence of at least 90%, or at least 95% sequence identity with one selected from SEQ ID NO: 75 to 94.

In some embodiments, the fusion proteins of the present invention comprise the human Angiotensin Converting Enzyme 2 (ACE2) receptor of the SARS-CoV-2 spike protein or the fragment thereof and a N-terminal signal peptide, and they do not comprise any of a polyhistidine tag, a streptavidin binding domain, a linker, or an oligomerization tag. In more preferred embodiment, said fusion proteins comprise an amino acid sequence selected from SEQ ID NO: 80, SEQ ID NO: 81, SEQ ID NO: 86, SEQ ID NO: 92 or SEQ ID NO: 93, or an amino acid of at least 90%, or at least 95% sequence identity with one selected from SEQ ID NO: 80, SEQ ID NO: 81, SEQ ID NO: 86, SEQ ID NO: 92 or SEQ ID NO: 93.

In some embodiments, the fusion protein of the present invention comprises the amino acid sequence selected from SEQ ID NO: 37, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, and SEQ ID NO: 100.

In some embodiments, the fusion protein of the present invention have an amino acid sequence of at least 90% sequence identity or at least 95% sequence identity with one of SEQ ID NO: 37, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, and SEQ ID NO: 100.

### III. NUCLEIC ACIDS, CLONING CELLS, AND EXPRESSION CELLS

The present invention also relates to nucleic acids comprising a nucleotide sequence encoding the fusion proteins described herein. The nucleic acid may be DNA or RNA. DNA comprising a nucleotide sequence encoding a fusion protein described herein typically comprises a promoter that is operably-linked to the nucleotide sequence. The promoter is preferably capable of driving constitutive or inducible expression of the nucleotide sequence in an expression cell of interest. Said nucleic acid may also comprise a selectable marker useful to select the cell containing said nucleic acid of interest. Useful selectable markers are well known by the skilled person. The precise nucleotide sequence of the nucleic acid is not particularly limiting so long as the nucleotide sequence encodes a fusion protein described herein. Codons may be selected, for example, to match the codon bias of an expression cell of interest (e.g., a mammalian cell such as a human cell) and/or for convenience during cloning. DNA may be a plasmid, for example, which may comprise an origin of replication (e.g., for replication of the plasmid in a prokaryotic cell).

In one embodiment described herein, the present invention refers to a nucleic acid comprising a nucleotide sequence encoding the fusion protein, a promoter operably linked to the nucleotide sequence and a selectable marker.

Various aspects of the present invention also relate to a cell comprising a nucleic acid comprising a nucleotide sequence that encodes a fusion protein as described herein. The cell may be an expression cell or a cloning cell. Nucleic acids are typically cloned in E. coli, although other cloning cells may be used.

If the cell is an expression cell, the nucleic acid is optionally a nucleic acid of a chromosome, i.e., wherein the nucleotide sequence is integrated into the chromosome, although then nucleic acid may be present in an expression cell, for example, as extrachromosomal DNA or vectors, such as plasmids, cosmids, phages, etc. The format of the vector should not be considered limiting.

In one embodiment described herein, the cell is typically an expression cell. The nature of the expression cell is not particularly limiting. Expression cells which may be used are prokaryotic cells such as E. coli and Bacillus spp. and eukaryotic cells such as yeast cells (e.g. S. cerevisiae, S. pombe, P. pastoris, K lactis, H polymorpha), insect cells (e.g. Sf9), fungal, plant cells or mammalian cells. Mammalian expression cells may allow for favorable folding, post-translational modifications, and/or secretion of a recombinant antibody or oligomeric recombinant antibody, although other eukaryotic cells or prokaryotic cells may be used as expression cells. Exemplary expression cells include CHO, TunaCHO, ExpiCHO, Expi293, BHK, NS0, Sp2/0, COS, C127, HEK, HT-1080, PER.C6, HeLa, and Jurkat cells. The cell may also be selected for integration of a vector, more preferably for integration of a plasmid DNA.

The fusion proteins of the present invention can be produced by appropriate transfection strategy of the nucleic acids comprising a nucleotide sequence that encodes the fusion proteins into mammalian cells. The skilled person is aware of the different techniques available for transfection of nucleic acids into the cell line of choice (lipofection, electroporation, etc). Thus, the choice of the mammalian cell line and transfection strategy should not be considered limiting. The cell line could be further selected for integration of the plasmid DNA.

Various aspects of the present invention also relate to a cell comprising the fusion proteins described herein.

### IV. COMPOSITIONS AND METHODS RELATED TO ASSAYS

Various aspects of the present invention relate to compositions comprising a fusion protein as described herein. In some embodiments, the composition may comprise a pharmaceutically-acceptable carrier and/or a pharmaceutically-acceptable excipient. The composition may be, for example, a vaccine.

Various embodiments of the present invention relate to a method of treating or preventing a SARS-CoV-2 infection in a human patient comprising administering to the patient a composition comprising a fusion protein as described herein. The term “preventing” as used herein refers to prophylaxis, which includes the administration of a composition to a patient to reduce the likelihood that the patient will become infected with SARS-CoV-2 relative to an otherwise similar patient who does not receive the composition. The term preventing also includes the administration of a composition to a group of patients to reduce the number of patients in the group who become infected with SARS-CoV-2 relative to an otherwise similar group of patients who do not receive the composition.

Various embodiments of the invention relate to a method of treating or preventing a SARS-CoV-2 infection in a human patient comprising administering to the patient a vaccine according to the embodiments described herein.

A patient may be infected with SARS-CoV-2, a patient may have been exposed to SARS-CoV-2, or a patient may present with an elevated risk for exposure to and/or infection with SARS-CoV-2.

In one embodiment described herein, the composition comprises the fusion protein of the present invention and a solid support.

In other embodiment, the composition comprises the fusion protein of the present invention and a solid support, wherein the fusion protein is covalently or non-covalently bound to the solid support. The term “non-covalently bound,” as used herein, refers to specific binding such as between an antibody and its antigen, a ligand and its receptor, or an enzyme and its substrate, exemplified, for example, by the interaction between streptavidin binding protein and streptavidin or an antibody and its antigen.

In other embodiment, the composition comprises the fusion protein of the present invention and a solid support, wherein the fusion protein is directly or indirectly bound to a solid support. The term “direct” binding, as used herein, refers to the direct conjugation of a molecule to a solid support, e.g., a gold-thiol interaction that binds a cysteine thiol of a fusion protein to a gold surface. The term “indirect” binding, as used herein, includes the specific binding of a fusion protein to another molecule that is directly bound to a solid support, e.g., a fusion protein may bind an antibody that is directly bound to a solid support thereby indirectly binding the fusion protein to the solid support. The term “indirect” binding is independent of the number of molecules between the fusion protein and the solid support so long as (a) each interaction between the daisy chain of molecules is a specific or covalent interaction and (b) a terminal molecule of the daisy chain is directly bound to the solid support.

A solid support may comprise a particle, a bead, a membrane, a surface, a polypeptide chip, a microtiter plate, or the solid-phase of a chromatography column.

A composition may comprise a plurality of beads or particles, wherein each bead or particle of the plurality of beads or particles are directly or indirectly bound to at least one fusion protein as described herein. A composition may comprise a plurality of beads or particles, wherein each bead or particle of the plurality of beads or particles are covalently or non-covalently bound to at least one fusion protein as described herein.

Various aspects of the embodiments relate to a kit for detecting the presence of antibodies against the fusion proteins of the present invention, a fragment thereof in a sample, said kit comprising a fusion protein and a solid support or composition as described herein.

The compositions and kits described herewith can be either for use in an assay or in compositions that are generated during the performance of an assay. Various aspects of the invention relate to a diagnostic medical device comprising a composition as described herein.

Various aspects of the invention relate to assays for detection of anti-SARS-CoV-2 antibodies.

An assay may be an assay for measuring the relative binding affinity of the fusion protein of the present invention to antibodies against SARS-CoV-2, or fragment thereof in a sample (e.g., relative to one or more control samples or standards). An assay may be an assay for measuring the relative binding affinity of the fusion protein of the present invention to any antibody against SARS-CoV-2, or fragment thereof (e.g., relative to one or more control samples or standards).

Assays typically feature a solid support that either allows for measurement, such as by nephelometry, UV/Vis/IR spectroscopy (e.g., absorption, transmission), fluorescence, chemiluminescence or phosphorescence spectroscopy, or surface plasmon resonance, or aids in the separation of components that directly or indirectly bind the solid support from components that do not directly or indirectly bind the solid support, or both. For example, an assay may include a composition comprising particles or beads and/or that aid in the mechanical separation of components that directly or indirectly bind the particles or beads.

Other exemplary assays that may include the fusion protein or the composition of the present invention includes but it is not limited to ELISA, viscoelastic tests such as Sonoclot, gel technologies, fluorescence assays, lateral flow, single molecule counting (SMC), and other point-of-care testing using any of these techniques.

The fusion proteins of the present invention will be further illustrated by the following non-limiting examples.

### EXAMPLES

### Example 1: Expression and Purification of Fusion Proteins of the Present Invention

All the fusion proteins of the present invention were produced recombinantly in mammalian cells (CHO or HEK cells). After expressed, said proteins were detected by SDS-PAGE under reducing and/or nor reducing conditions (data not shown).

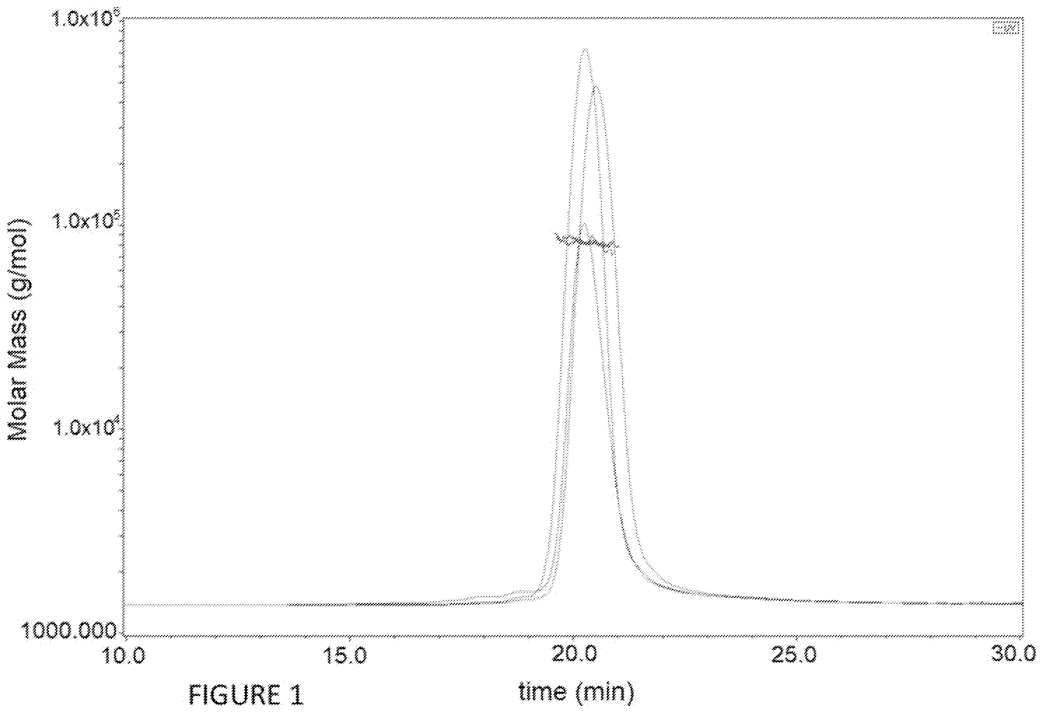

In order to assess the assembly and aggregation of the full length size (FLS) and ACE2 proteins, size exclusion multi-angle light scattering (SEC-MALS) was employed. SEC-MALS provides information about the molecular weight and the polydispersity (Mw/Mn) of the sample. FIG. 1 provides SEC-MALS data for fusion proteins containing ACE2 (SEQ ID NO: 75, 77 and 79). Generating monodisperse products with the proper oligomerization is important for consistency and reproducibility of diagnostic assays.

### Example 2: Confirmation of Functionality of ACE2 Fusion Proteins Using Commercial RBD Protein

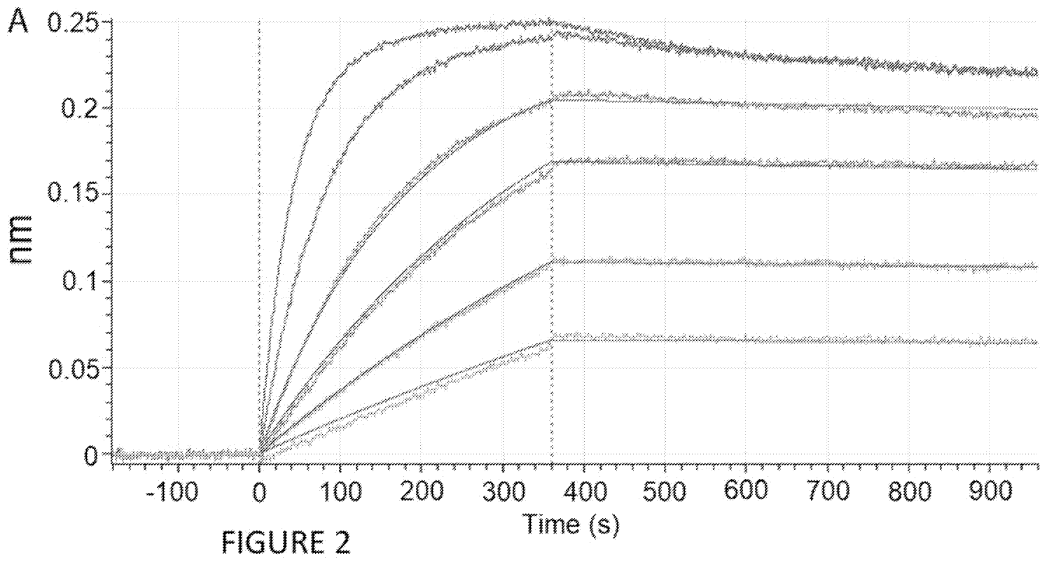

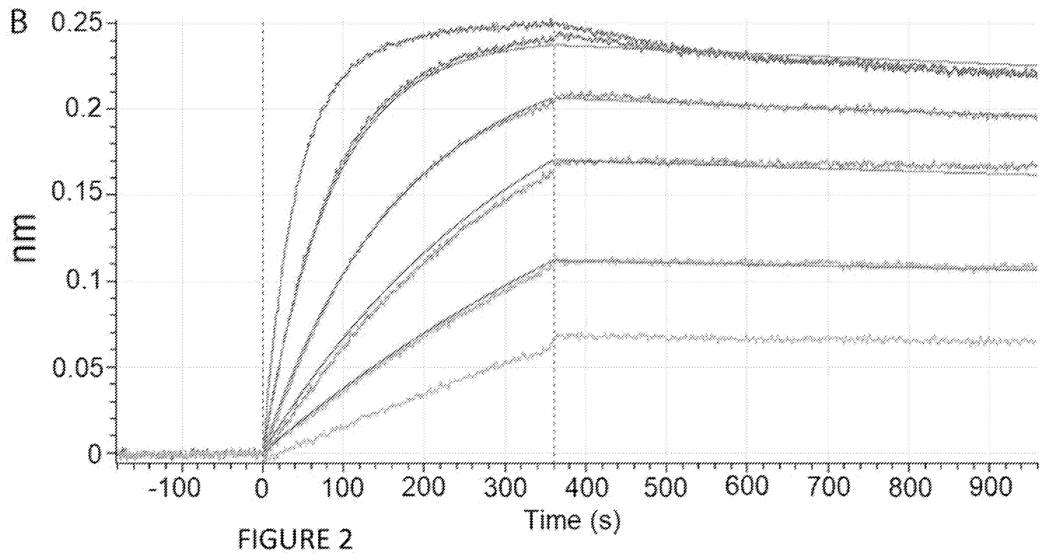

In order to evaluate the ACE2 fusion proteins, the full length size (FLS) spike and S1 proteins first confirmation that the ACE2 (SEQ ID NO: 77) fusion protein was functional capable of binding receptor binding domain (RBD) of the spike protein was required. This was done using a commercial RBD protein (FIG. 2A). Subsequently, the pxAM-FLS-SSAS (SEQ ID NO: 41) was substituted for the commercial RBD protein (FIG. 2B). In both cases, there was consistent nanomolar binding detected. Confirming the functionality of the biotinylated pxEBN4-ACE2 (SEQ ID NO: 77) with both RBD and FLS recombinant proteins allowed for extension of the octet ACE2 binding characterization to different versions of FLS fusion proteins.

### Example 3: Characterization and Down-Selection of FLS Fusion Proteins

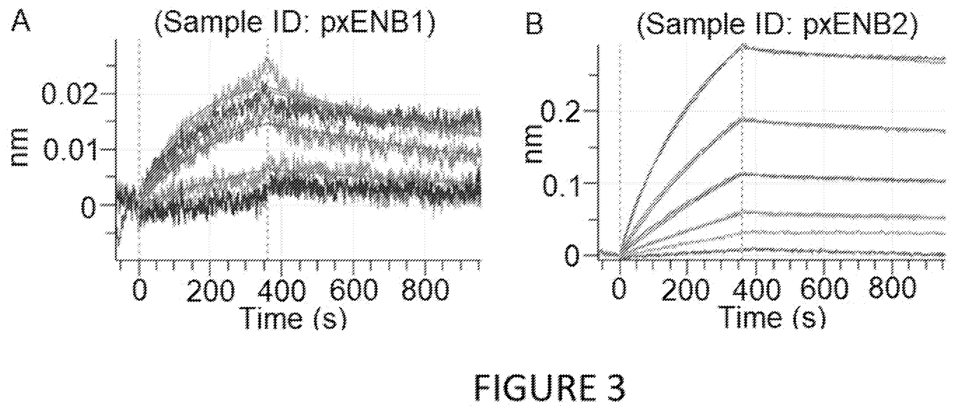

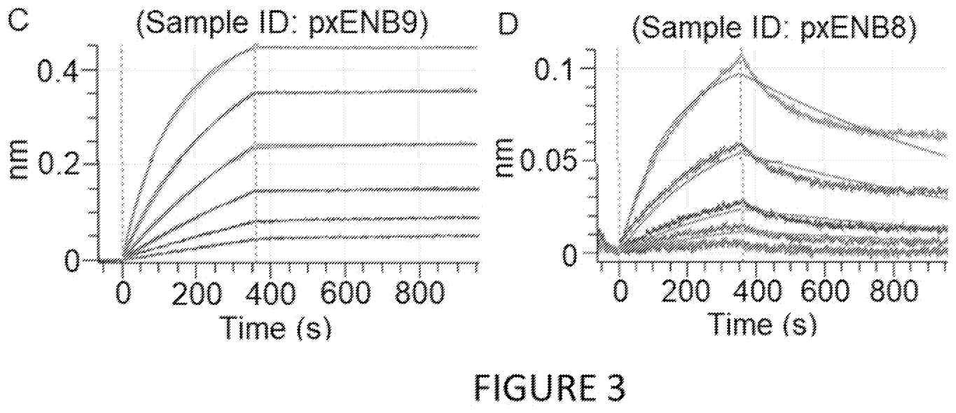

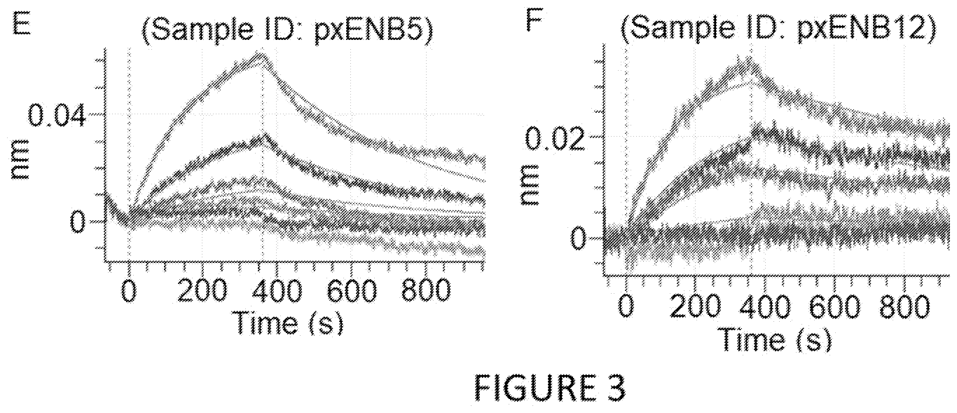

Six different FLS fusion proteins were expressed, purified, and characterized with His tags at either the N or C terminus and utilizing either no stabilizing trimerization domain or trimerization domains. All six proteins were able to bind ACE2 in the octet assay (FIG. 3, Table 1). In general constructs with no stabilizing trimerization domain were the highest expressed followed by constructs with the short T4 trimerization domain and constructs with the tetranectin trimerization domain were the lowest expressed. Either N or C terminally tagged constructs with the short T4 trimerization domain exhibited tighter affinity to ACE2 than constructs with the tetranectin trimerization domain, constructs without a trimerization domain showed the weakest affinity. From these studies, pxENB9-FLS (SEQ ID NO:46) was selected for further analysis. pxENB9-FLS had the tightest binding and was well produced in this comparison study.

TABLE 1
Summary of Data for Down-Selection of FLS Constructs from
initial transient transfection including estimated yield/1
L of culture media, binding affinity (KD (M)) with ACE2.

| | Yield | Ranked Binding to |
|---|---|---|
| Fusion protein | (mg/1 L) | pxEBN4-ACE2-AAA-His |
| pxAM-FLS-SSAS | 0.26 | ++ |
| (SEQ ID NO: 41) | | (5.24E−10M) |
| pxENB1-FLS | 1.11 | + |
| (SEQ ID NO: 42) | | (3.9E−9M) |
| pxENB2-FLS | 1.28 | ++ |
| (SEQ ID NO: 43) | | (1.1E−9M) |
| pxENB5-FLS | 3.36 | + |
| (SEQ ID NO: 44) | | (1.66E−8M) |
| pxENB8-FLS | 0.78 | ++ |
| (SEQ ID NO: 45) | | (6.51E−9M) |
| pxENB9-FLS | 2.21 | +++ |
| (SEQ ID NO: 46) | | (<1E−12M) |
| pxENB12-FLS | 2.27 | + |
| (SEQ ID NO: 47) | | (3.19E−9M) |

### Example 4: Rabbit Polyclonal Antibodies Raised Against S1 Fusion Protein are Capable of Inhibiting the Binding of FLS and ACE2

Further BioLayer Interferometry (BLI) was utilized to demonstrate that polyclonal antibodies (pAb) raised against S1 fusion protein interfere with the binding of ACE2 and FLS. Additionally, the inclusion of streptavidin binding site (SBP) at the C-terminus of ACE2 was able to bind streptavidin tips while ACE without the SBP fusion partner showed no response.

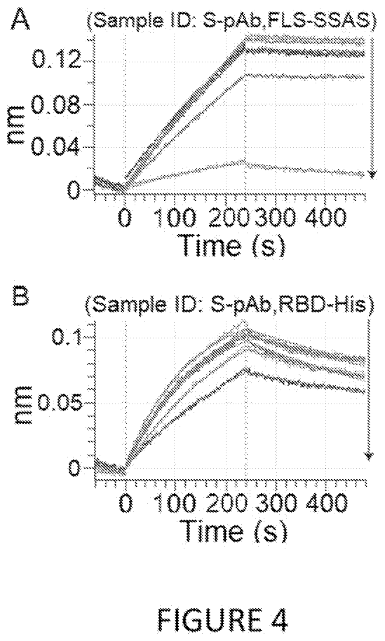

FIG. 4 shows BLI sensorgrams with biotinylated pxEBN4-ACE2 (SEQ ID NO:77) was loaded on streptavidin tips followed by either (A) pxAM-FLS-SSAS (4 nM) (SEQ ID NO: 41) or (B) pxENB17-RBD (12 nM) preincubated with a serial dilution of Sino Biological Rabbit anti-S1 pAb. In the presence of the increased pAb concentration there is a decreased response, illustrated by the downward arrow, indicating decreased binding between the RBD and FLS. The FLS showed increased sensitivity to the pre-incubation with the pAb relative to the RBD.

### Example 5: BLI Sensorgrams Show Human ACE2 Receptor-Streptavidin Tip Loading and Binding RBD

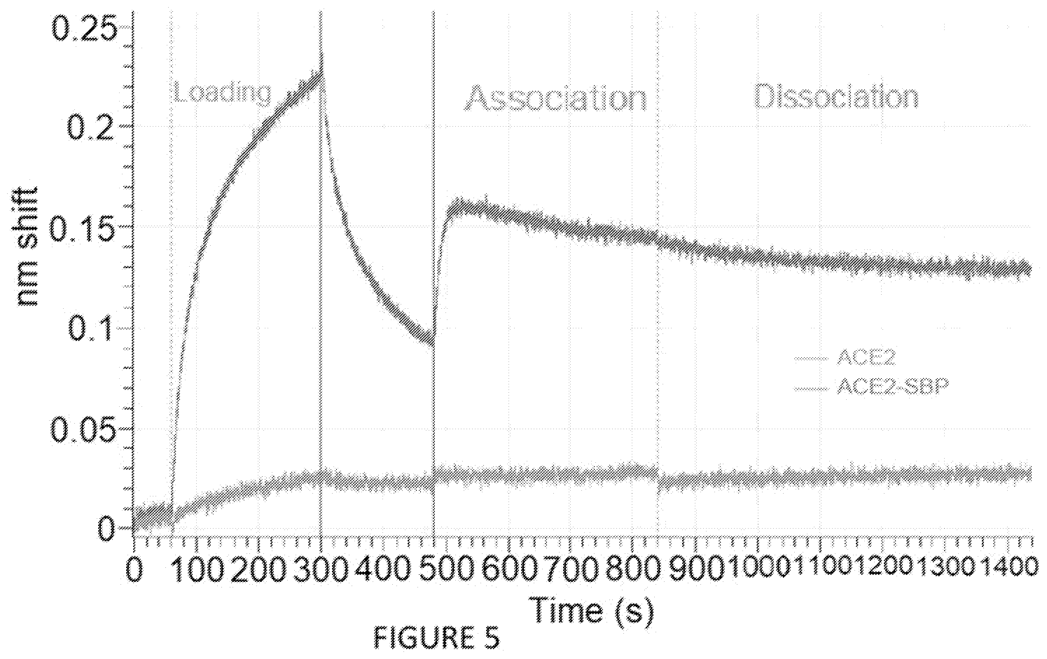

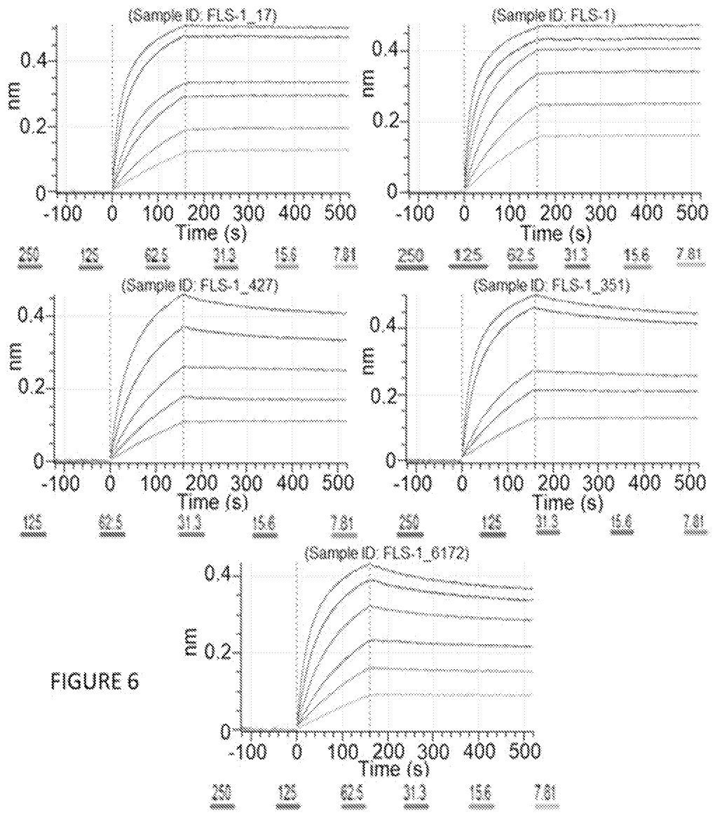

FIG. 5 shows a comparison of binding curves of immobilized ACE2 (gray) and ACE2-SBP (blue) loaded on streptavidin biosensors at 35.85 μg/mL and associating with recombinant SARS-CoV-2 RBD (pxENB17-RBD) at a concentration of 6.25 nM.

### Example 6: Characterization of Mutant FLS Constructs

FLS mutant constructs were expressed and purified based on the sequences for emergent SARS-CoV-2 variants currently identified worldwide (CDC website: https://www.cdc.gov/coronavirus/2019-ncov/variants/variant-info.html #Concern) pxENB9-FLS (SEQ ID NO: 46), the top-performing FLS construct, was used as a template for these mutants. The five different FLS proteins with His tags at the C− terminus and utilizing the stabilization trimerization T4 phage head fibrinitin domain were expressed, purified, and characterized. All five proteins were able to bind recombinant hACE2 in the ELI assay (FIG. 6, Table 2). The UK and Brazil Variants (FLS-B.1.1.7 and FLS-P.1, respectively.) have shown the tightest binding to rhACE2. All five proteins were expressed with similar yields.

TABLE 2
Summary of Data for Down-Selection of FLS Constructs from initial
transient transfection including yield/1 L of culture media, binding
affinity (K_{D} (M)) with ACE2, and final purity.

| | | Ranked Binding to | |
|---|---|---|---|
| | Yield | pxEBN4-ACE2- | |
| Construct | (mg/1 L) | AAA-His, K_{D} | Purity |
| UK, Alpha, | 3.01 | ++ | >99% Total |
| pxENB9-FLS | | (1.16E−12M) | 93.2% Monomer |
| (SEQ ID NO: 46) | | | (by SDS-PAGE) |
| SA, Beta, | 3.51 | ++ | >99% Total |
| pxENB9-FLS- | | (1.57E−09M) | 95.0% Monomer |
| B.1.351 | | | (by SDS-PAGE) |
| (SEQ ID NO: 98) | | | |
| India, Delta, | 4.10 | +++ | >99% Total |
| pxENB9-FLS- | | (2.55E−09M) | 96.2% Monomer |
| B.1.617.2 | | | (by SDS-PAGE) |
| (SEQ ID NO: 99) | | | |
| US/CA, Epsilon, | 5.06 | ++ | >99% Total |
| pxENB9-FLS- | | (1.30E−09M) | 95.1% Monomer |
| B.1.427 | | | (by SDS-PAGE) |
| (SEQ ID NO: | | | |
| 100) | | | |
| Brazil, Gamma, | 3.50 | +++ | >99% Total |
| pxENB9-FLS-P.1 | | (1.59E−12M) | 95.1% Monomer |
| (SEQ ID NO: 37) | | | (by SDS-PAGE) |

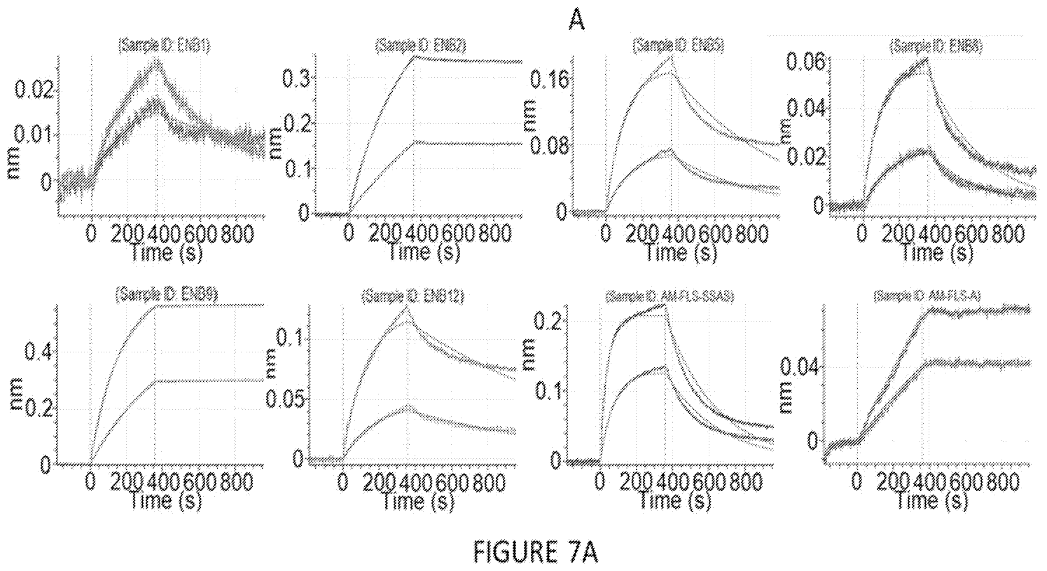

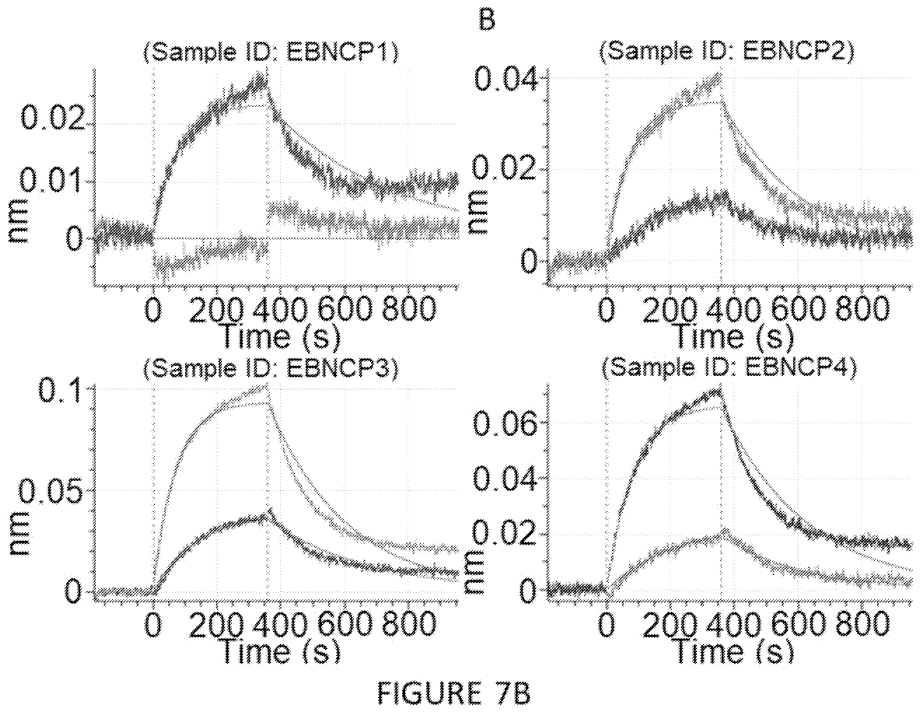

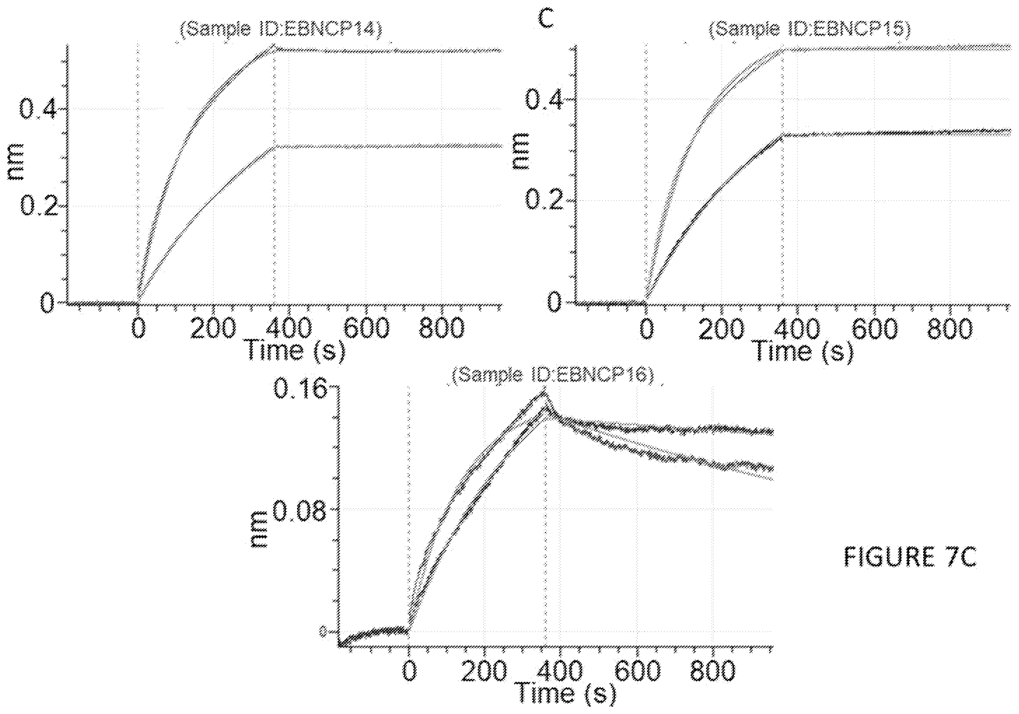

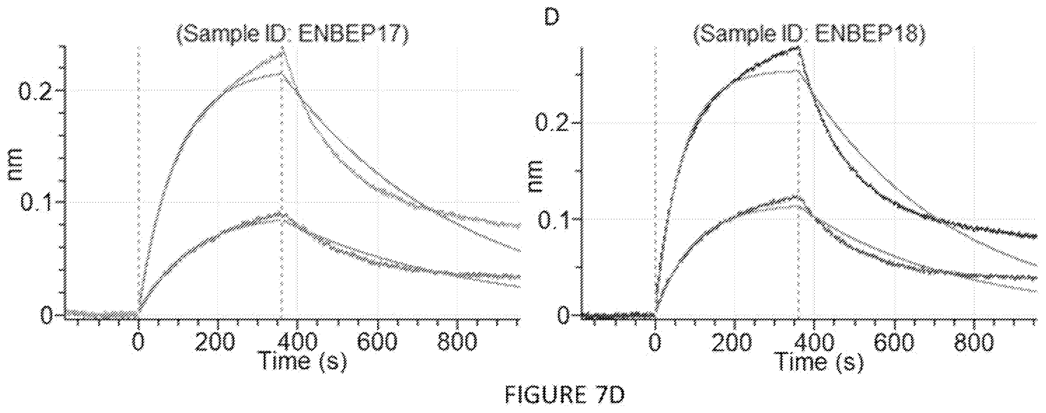

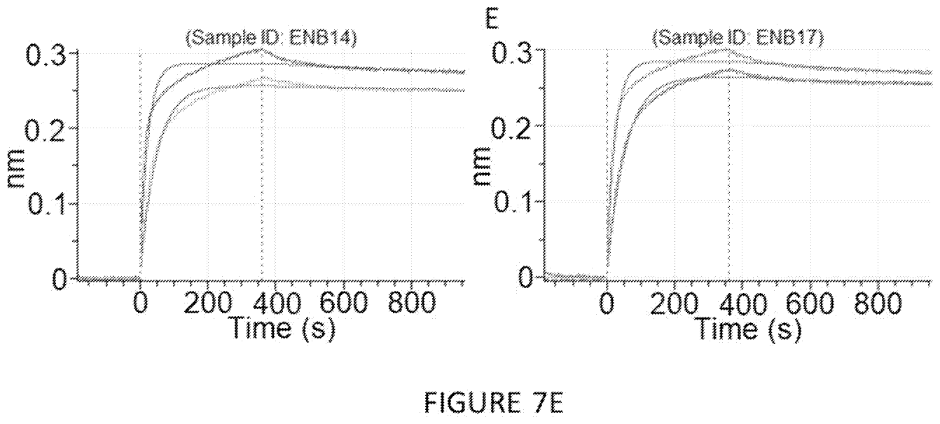

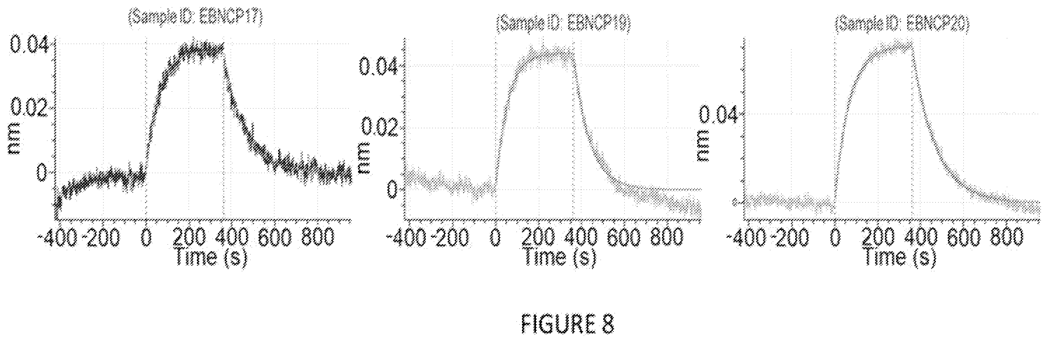

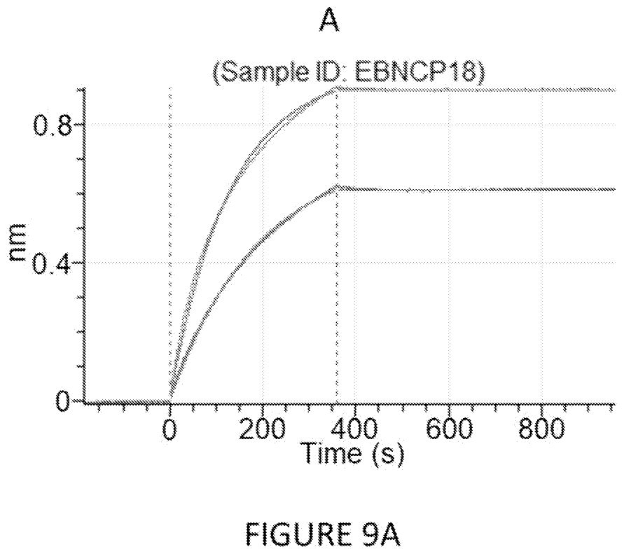

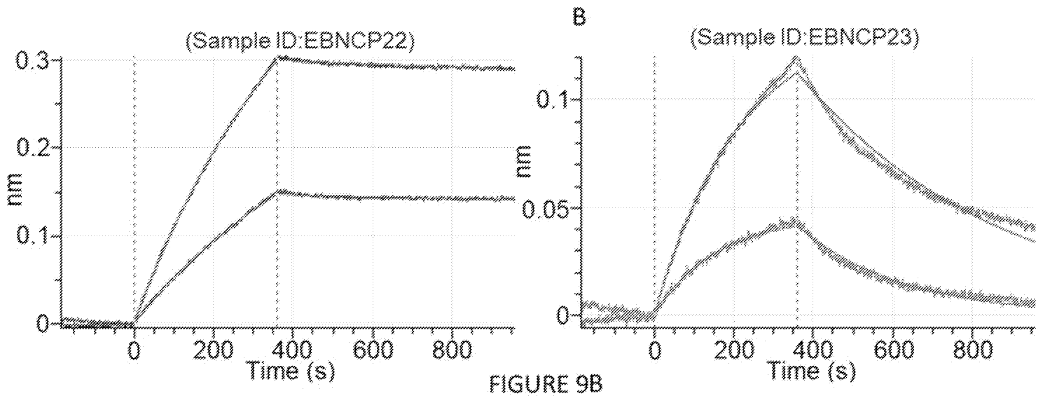

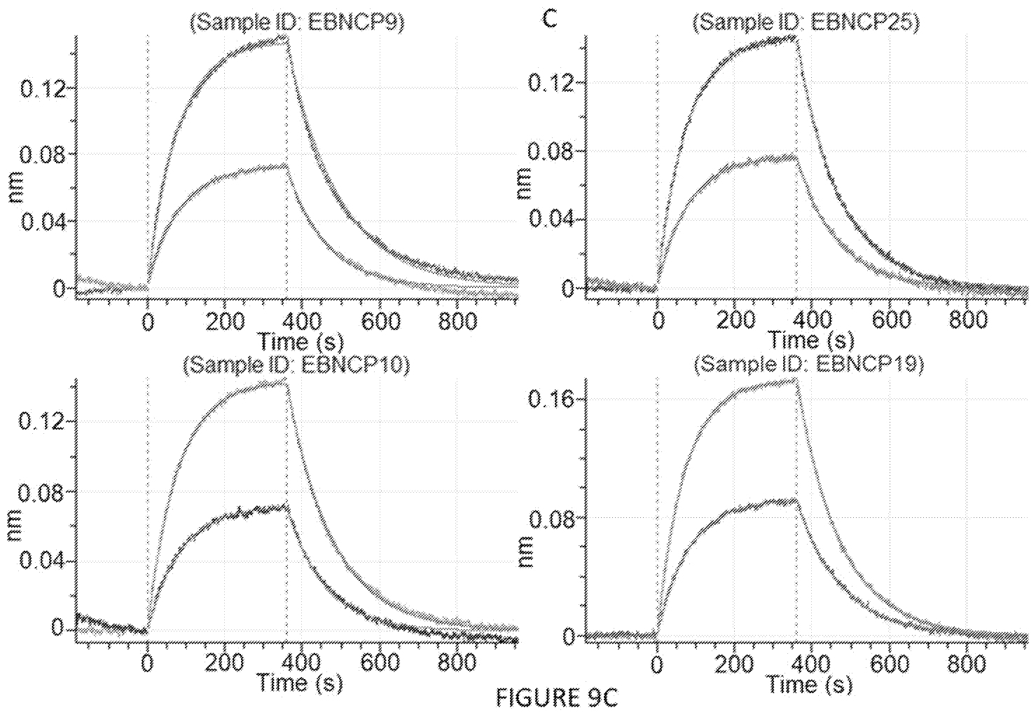

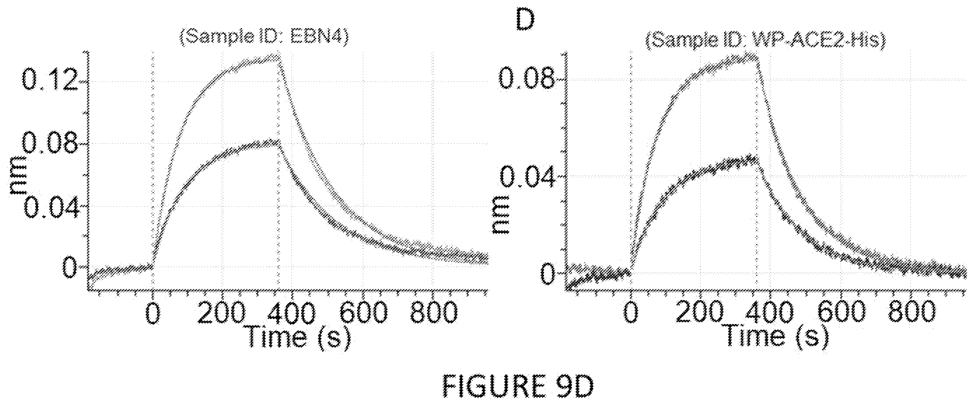

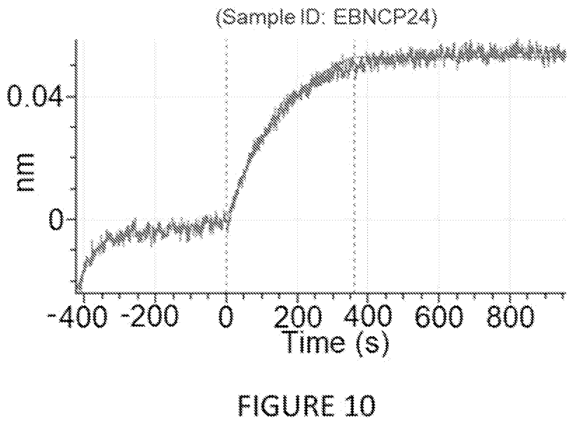

pxEBN4-ACE2-AAA-His biotinylated at a 2:1 molar ratio (biotin:protein; assuming monomerization of ACE2 construct) and loaded at a concentration of 35.86 μg/mL onto SA Octet Tips. All FLS/S1/S2/RBD constructs were separately associated to bt-ACE2, at 100 nM and 33.33 nM (accounting for dimerization and trimerization domains). Preliminary KD and response data can be found in Table 3 is used to compare constructs. As shown in FIG. 7: A. Initial FLS Constructs. B. SARS-CoV-2 S1 Constructs. C. SARS-CoV-2 FLS Constructs with Alternative Signal Peptides. 0. SARS-CoV-2 Nuclear Capsid—Spike Fusions. E. SARS-CoV-2 RED Constructs. A. Initial FLS Constructs. B. SARS-CoV-2 S1 Constructs. C. SARS-CoV-2 FLS Constructs with Alternative Signal Peptides. 0. SARS-CoV-2 Nuclear Capsid—Spike Fusions. E. SARS-CoV-2 RED Constructs.

TABLE 3
Summary of Data for Down-Selection of FLS/S1/S2/RBD Constructs from initial transient
transfection, including yield from culture media, binding affinity (KD (M)) with
biotinylated ACE2, in-assay binding response, and final purity. Biotinylated ACE2
was loaded onto SA Octet tips, then FLS/S1/S2/RBD was associated and dissociated.

| | | | Response, | |
|---|---|---|---|---|
| | | | ranked from | |
| | | | greatest to | |
| | | Ranked Binding | least shift in | |
| | | to bt-pxEBN4- | Wavelength | |
| Construct | Yield | ACE2-AAA-His | (nm) | Purity |
| pxENB1-His-TEV-FLS-TN | See Table 1 | + | − | See |
| (SEQ ID NO: 42) | | (6.92E−08) | (0.0257) | Table 1 |
| pxENB2-His-TEV-FLS-CT | See Table 1 | ++ | ++ | See |
| (SEQ ID NO: 43) | | (4.50E−10) | (0.3438) | Table 1 |
| pxENB5-his-TEV-FLS | See Table 1 | + | + | See |
| (SEQ ID NO: 44) | | (1.95E−08) | (0.1848) | Table 1 |
| pxENB8-FLS-TN-His | See Table 1 | + | − | See |
| (SEQ ID NO: 45) | | (1.24E−08) | (0.0591) | Table 1 |
| pxENB9-FLS-CT-His | See Table 1 | +++ | +++ | See |
| (SEQ ID NO: 46) | | (1.00E−12) | (0.5548) | Table 1 |
| pxENB12-FLS-His | See Table 1 | + | + | See |
| (SEQ ID NO: 47) | | (1.31E−08) | (0.1266) | Table 1 |
| pxAM-FLS-SSAS-TB-His | See Table 1 | + | ++ | See |
| (SEQ ID NO: 41) | | (5.62E−09) | (0.2219) | Table 1 |
| pxAM-FLS-A-TB-His | 0.718 mg/ | +++ | − | Not |
| (SEQ ID NO: 40) | 150 mL | (7.09E−12) | (0.0651) | Available |
| pxEBNCP1-His-TEV-S1 | 0.0435 mg/ | + | − | 61.4% |
| (SEQ ID NO: 67) | 960 mL | (2.28E−08) | (0.0268) | |
| pxEBNCP2-tPa-His-TEV-S1 | 0.0285 mg/ | + | − | 50.2% |
| (SEQ ID NO: 68) | 960 mL | (3.42E−08) | (0.0391) | |
| pxEBNCP3-S1-His | 0.0713 mg/ | + | + | 77.4% |
| (SEQ ID NO: 69) | 960 mL | (3.96E−08) | (0.1004) | |
| pxEBNCP4-tPa-S1-His | 0.212 mg/ | + | − | 77.2% |
| (SEQ ID NO: 70) | 960 mL | (4.50E−08) | (0.0703) | |
| pxEBNCP14-tPa-FLS-CT- | 0.25 mg/ | +++ | +++ | 83% |
| His | 750 mL | (6.13E−12) | (0.529) | |
| (SEQ ID NO: 48) | | | | |
| pxEBNCP15-mIgGHC- | 0.233 mg/ | +++ | +++ | 95% |
| FLS-CT-His | 750 mL | (1.00E−12) | (0.4921) | |
| (SEQ ID NO: 49) | | | | |
| pxEBNCP16-IL2-FLS-CT- | 0.0248 mg/ | + | + | 30%* |
| His | 750 mL | (1.46E−09) | (0.145) | |
| (SEQ ID NO: 50) | | | | |
| pxENBEP17_TPA_Nuc(1- | 7.52 mg/ | + | ++ | Not |
| 182)_Spike (15- | 1000 mL | (2.69E−08) | (0.2318) | Available |
| 1216, GAAS, PP)_10xHis | | | | |
| pxENBEP18_Spike_Nuc(1- | 8.91 mg/ | + | ++ | Not |
| 182)_10xHis | 1000 mL | (2.16E−08) | (0.2768) | Available |
| pxENB14-His-TEV-RBD | 3.7 mg/ | ++ | ++ | Not |
| | 100 mL | (1.56E−10) | (0.3044) | Available |
| pxENB17-RBD-His | 3.0 mg/ | ++ | ++ | Not |
| | 100 mL | (2.42E−10) | (0.3008) | Available |

FLS constructs were loaded at 33.3 nM (assuming trimerization) onto SA Octet Tips. ACE2 was associated to FLS constructs at. 33 nM (accounting for dimerization). Preliminary KD and response data can be found in Table 4 is used to compare constructs.

TABLE 4
Summary of Data for Down-Selection of FLS Constructs with Streptavidin
tags from initial transient transfection, including yield from culture media,
binding affinity [K_{D} (M)] with ACE2, in-assay binding response,
and final purity. FLS was loaded onto SA Octet tips by their respective
tags, then ACE2 was associated and dissociated.

| | | | Response, | |
|---|---|---|---|---|
| | | | ranked from | |
| | | Ranked | greatest to | |
| | | Binding | least shift in | |
| | | to pxEBN4- | Wavelength | |
| Construct | Yield | ACE2-AAA-His | (nm) | Purity |
| pxENBCP17-FLS-CT- | 2.77 mg/ | + | − | 92% |
| His-SBP | 750 mL | (5.15E−08) | (0.0382) | |
| (SEQ ID NO: 51) | | | | |
| pxENBCP19-FLS-CT- | 1.89 mg/ | + | − | 93% |
| His-StrepTagII | 750 mL | (9.08E−08) | (0.042) | |
| (SEQ ID NO: 53) | | | | |
| pxENBCP20-FLS-CT- | 1.66 mg/ | + | − | 95% |
| His-TST | 750 mL | (5.16E−08) | (0.0719) | |
| (SEQ ID NO: 54) | | | | |

A recombinant RED was biotinylated at a 1:1 molar ratio (biotin:protein) and loaded at a concentration of 1.68 μg/mL onto SA Octet Tips. All ACE2 constructs were separately associated to bt-ACE2, at 100 nM and 33.33 nM (accounting for multimerization domains). Preliminary KD and response data can be found in Table 5 is used to compare constructs. Data found in Table 6 is not shown but follows the same set-up with the only difference being that bt-pxENE9-FLS-CT-His was loaded onto SA Octet Tips. This data cannot be relied upon to provide significantly accurate KD data. A. ACE2 Constructs with p53 Subunit fusions. E. ACE2 Constructs with mIgG Fc Fusions. C. ACE2 Constructs with Alternative Signal Peptides. D. ACE2 Native Constructs with AAA mutation.

TABLE 5
Summary of Data for Down-Selection of ACE2 Constructs from initial
transient transfection, including yield from culture media, binding
affinity (K_{D} (M)) with biotinylated ACE2, in-assay binding response,
and final purity. Biotinylated RBD was loaded onto SA Octet
tips, then ACE2 was associated and dissociated.

| | | Response, |
|---|---|---|
| | | ranked from |
| | Ranked | greatest to |
| | Binding to | least shift in |
| | bt-pxENB17- | Wavelength |
| Construct | RBD-His | (nm) |
| pxEBNCP18_hACE2-AAA_p53 | +++ | +++ |
| (SEQ ID NO: 85) | (1.00E−12) | (0.9023) |
| pxEBNCP22_hACE2_3A-mIgG(h23) | ++ | ++ |
| (SEQ ID NO: 89) | (9.53E−10) | (0.299) |
| pxENBCP23_hACE2_3A-mIgG(23) | + | + |
| (SEQ ID NO: 90) | (2.45E−08) | (0.1187) |
| pxEBNCP9_hIL2-hACE2(18-615 | + | + |
| (SEQ ID NO: 80) | (4.51E−08) | (0.1496) |
| pxEBNCP10_tPa-hACE2(18-615)-AAA | + | + |
| (SEQ ID NO: 81) | (6.08E−08) | (0.1445) |
| pxEBNCP19_mIgGHC_hACE2-3A | + | + |
| (SEQ ID NO: 86) | (5.62E−08) | (0.1729) |
| pxEBNCP25_hACE2-AAA(1-615) | + | + |
| (SEQ ID NO: 92) | (7.33E−08) | (0.1457) |
| pxEBN4-ACE2-AAA-His | + | + |
| (SEQ ID NO: 77) | (3.26E−08) | (0.1372) |
| pxWP-ACE2-His | + | + |
| (SEQ ID NO: 75) | (4.71E−08) | (0.0896) |

TABLE 6
Summary of Data for Down-Selection of ACE2 Constructs from initial
transient transfection, including yield from culture media, binding
affinity (K_{D} (M)) with biotinylated ACE2, in-assay binding response,
and final purity. Biotinylated FLS was loaded onto SA Octet
tips, then ACE2 was associated and dissociated.

| | | | Response, | |
|---|---|---|---|---|
| | | | ranked from | |
| | | Ranked | greatest to | |
| | | Binding to | least shift in | |
| | | bt-pxENB9- | Wavelength | |
| Construct | Yield | FLS-CT-His | (nm) | Purity |
| pxEBNCP18_hACE2-AAA_p53 | 5.78 mg/ | +++ | +++ | 92% |
| (SEQ ID NO: 85) | 240 mL | (1.00E−12) | (0.5447) | |
| pxEBNCP22_hACE2_3A- | 7.93 mg/ | +++ | + | 89% |
| mIgG(h23) | 240 mL | (1.00E−12) | (0.1456) | |
| (SEQ ID NO: 89) | | | | |
| pxENBCP23_hACE2_3A- | 2.57 mg/ | + | − | 48%* |
| mIgG(23) | 240 mL | (3.87E−08) | (0.066) | |
| (SEQ ID NO: 90) | | | | |
| pxEBNCP9_hIL2-hACE2(18- | 14.4 mg/ | + | + | 98% |
| 615) | 240 mL | (3.05E−08) | (0.0828) | |
| (SEQ ID NO: 80) | | | | |
| pxEBNCP10_tPa-hACE2(18- | 21.7 mg/ | + | − | 98% |
| 615)-AAA | 240 mL | (9.39E−08) | (0.0729) | |
| (SEQ ID NO: 81) | | | | |
| pxEBNCP19_mIgGHC_hACE2- | 21.5 mg/ | + | + | 98% |
| 3^{a} | 240 mL | (4.24E−08) | (0.0985) | |
| (SEQ ID NO: 86) | | | | |
| pxEBNCP25_hACE2-AAA(1- | 20.4 mg/ | + | + | 98% |
| 615) | 240 mL | (4.26E−08) | (0.0853) | |
| (SEQ ID NO: 92) | | | | |
| pxEBN4-ACE2-AAA-His | 11.0 mg/ | + | + | Not |
| (SEQ ID NO: 77) | 150 mL | (3.66E−08) | (0.0966) | Available |
| pxWP-ACE2-His | 8.04 mg/ | + | − | Not |
| (SEQ ID NO: 75) | 150 mL | (4.21E−08) | (0.0676) | Available |

*49% of the bands corresponded to a lower MW version (~120 kDa). Reducing the sample results in ~95% of the protein in the lower 120 kDa band.

ACE2 constructs were loaded at a concentration of 33.3 nM by their respective Streptavidin tags onto SA Octet Tips. pxENB9-FLS-CT-His was associated to the ACE2 constructs at 33.33 nM (accounting for multimerization domains). Preliminary KD and response data can be found in Table 7 is used to compare constructs. Data found in Table 8 is not shown but follows the same set-up with the only difference being that pxENB17-RBD-His was associated to ACE2 constructs. The only data shown is that of pxEBNCP24-hACE2-AAA-p53-SBP because all the other ACE2 constructs with Streptavidin tags performed negligibly.

TABLE 7
Summary of Data for Down-Selection of ACE2 Constructs with Streptavidin
tags from initial transient transfection, including yield from culture media,
binding affinity (K_{D} (M)) with ACE2, in-assay binding response, and
final purity. ACE2 was loaded onto SA Octet tips by their respective
tags, then FLS was associated and dissociated.

| | | | Response, | |
|---|---|---|---|---|
| | | | ranked from | |
| | | Ranked | greatest to | |
| | | Binding to | least shift in | |
| | | pxENB9- | Wavelength | |
| Construct | Yield | FLS-CT-His | (nm) | Purity |
| pxEBNCP24_hACE2-AAA- | 9.87 mg/ | +++ | − | 97 |
| p53-SBP | 240 mL | (1.00E−12) | (0.0508) | |
| (SEQ ID NO: 91) | | | | |
| pxEBNCP21_hACE2-3A- | 5.07 mg/ | − | − | 92 |
| mIgG1(h23)TST | 240 mL | (1.00E−07) | (−0.02292) | |
| (SEQ ID NO: 88) | | | | |
| pxEBNCP15_hACE2-AAA- | 18.4 mg/ | − | − | 97 |
| StrepTagII | 240 mL | (4.71E−06) | (0.0043) | |
| (SEQ ID NO: 84) | | | | |
| pxEBNCP14_hACE2_3ATST | 19.4 mg/ | − | − | 98 |
| (SEQ ID NO: 83) | 240 mL | (1.91E−04) | (0.0027) | |
| pxEBNCP13_hACE2-AAA- | 28.4 mg/ | − | − | 98 |
| SBP | 240 mL | (1.05E−05) | (0.0019) | |
| (SEQ ID NO: 82) | | | | |
| pxEBN9-ACE2-AAA-SBP- | 8.4 mg/ | − | − | Not |
| His | 150 mL | (1.34E−05) | −0.00064) | Available |
| (SEQ ID NO: 79) | | | | |

TABLE 8
Summary of Data for Down-Selection of ACE2 Constructs with
Streptavidin tags from initial transient transfection, including
yield from culture media, binding affinity (K_{D} (M)) with
ACE2, in-assay binding response, and final purity. ACE2
was loaded onto SA Octet tips by their respective tags,
then RBD was associated and dissociated.

| | | Response, |
|---|---|---|
| | | ranked from |
| | | greatest to |
| | Ranked Binding | least shift in |
| | to pxENB17- | Wavelength |
| Construct | RBD-His | (nm) |
| pxEBNCP24_hACE2-AAA- | ++ | − |
| p53-SBP | (2.31E−10) | (0.028) |
| (SEQ ID NO: 91) | | |
| pxEBNCP21_hACE2-3A- | + | − |
| mIgG1(h23)TST | (5.65E−08) | (0.0063) |
| (SEQ ID NO: 88) | | |
| pxEBNCP15_hACE2-AAA- | − | − |
| StrepTagII | (1.10E−07) | (0.0062) |
| (SEQ ID NO: 84) | | |
| pxEBNCP14_hACE2_3ATST | − | − |
| (SEQ ID NO: 83) | (4.48E−07) | (0.0089) |
| pxEBNCP13_hACE2-AAA- | − | − |
| SBP | (4.01E−07) | (0.0102) |
| (SEQ ID NO: 82) | | |
| pxEBN9-ACE2-AAA-SBP- | − | − |
| His | (3.26E−05) | (0.0002) |
| (SEQ ID NO: 79) | | |

| SEQ ID NOS | Sequence (5′ to 3′) | Comments |
|---|---|---|
| SEQ ID NO: 1 | MFVFLVLLPLVSSQ | SARS-Cov-2 spike |
| | | protein Endogenous |
| | | signal peptide |
| SEQ ID NO: 2 | MKRGLCCVLLLCGAVEVSPS | Tissue plasminogen |
| | | activator signal |
| | | peptide |
| SEQ ID NO: 3 | MEWSWVFLFFLSVTTGVHS | Murine IgG HC |
| | | signal peptide |
| SEQ ID NO: 4 | MYRMQLLSCIALSLAL | Human IL2 signal |
| | | peptide |
| SEQ ID NO: 5 | MSSSSWLLLSLVAVTAA | Angiotensin- |
| | | converting enzyme 2 |
| | | signal peptide |
| SEQ ID NO: 6 | SSAS | Furin cleavage site |
| | | mutation |
| SEQ ID NO: 7 | GAAS | Furin cleavage site |
| | | mutation |
| SEQ ID NO: 8 | RRAR | Furin cleavage site |
| | | mutation |
| SEQ ID NO: 9 | LVPRGS | Thrombin cleavage |
| | | site |
| SEQ ID NO: 10 | ENLYFQ | TEV cleavage site |
| SEQ ID NO: 11 | GGGS | Flexible linker |
| SEQ ID NO: 12 | GGSG | Flexible linker |
| SEQ ID NO: 13 | GGSGG | Flexible linker |
| SEQ ID NO: 14 | LEVLFQGP | HRV-3C Protease |
| | | Cleavage Site |
| SEQ ID NO: 15 | GGGP | Kinked linker |
| SEQ ID NO: 16 | HHHHHH | Histidine tag (6x) |
| SEQ ID NO: 17 | HHHHHHHH | His tag (8x) |
| SEQ ID NO: 18 | HHHHHHHHHH | His tag (10x) |
| SEQ ID NO: 19 | GSGYIPEAPRDGQAYVRKDGEWVLLSTELG | T4 fibritin foldon |
| | | domain |
| SEQ ID NO: 20 | GYIPEAPRDGQAYVRKDGEWVLLSTEL | Short T4 fibritin |
| | | foldon domain |
| SEQ ID NO: 21 | WKQSVELAKKDSLYKDAMQYASESKDTELAEELLQWELQEEKREC | Clathrin |
| | FGACLFTCYDLLRPDVVLELAWRHNIMDFAMPYFIQVMKEYLTKV | trimerization |
| | DKLDASESLRKEEE | domain |
| SEQ ID NO: 22 | ASSGVRLWATRQAMLGQVHEVPEGWLIFVAEQEELYVRVQNGERK | Human Collagen |
| | VQLEARTPLPR | Trimerization Domain |
| SEQ ID NO: 23 | RMKQLEDKVEELLSKNYHLENEVARLKKLVGER | GCN4 based |
| | | isoleucine zipper |
| SEQ ID NO: 24 | LKSRLDTLSQEVALLKEQQALQTVCL | Tetranectin |
| | | trimerization domain |
| SEQ ID NO: 25 | KPLDGEYFTLQIRGRERFEMFRELNEALELKDAQAGKEPG | p53 tetramerization |
| | | domain |
| SEQ ID NO: 26 | VPEVSSVFIFPPKPKDVLTITLTPKVTCVVVDISKDDPEVQFSWE | Murine IgG1-Fc |
| | VDDVEVHTAQTQPREEQFNSTERSVSELPIMHQDWLNGKEFKCRV | (without hinge) |
| | NSAAFPAPIEKTISKTKGRPKAPQVYTIPPPKEQMAKDKVSLTCM | |
| | ITDFFPEDITVEWQWNGQPAENYKNTQPIMDTDGSYFVYSKLNVQ | |
| | KSNWEAGNTFTCSVLHEGLHNHHTEKSLSHSPGK | |
| SEQ ID NO: 27 | VPRDCGCKPCICTVPEVSSVFIFPPKPKDVLTITLTPKVTCVVVD | Murine IgG1-Fc |
| | ISKDDPEVQFSWFVDDVEVHTAQTQPREEQFNSTERSVSELPIMH | Dimerization domain |
| | QDWLNGKEFKCRVNSAAFPAPIEKTISKTKGRPKAPQVYTIPPPK | |
| | EQMAKDKVSLTCMITDFFPEDITVEWQWNGQPAENYKNTQPIMDT | |
| | DGSYFVYSKLNVQKSNWEAGNTFTCSVLHEGLHNHHTEKSLSHSP | |
| | GK | |
| SEQ ID NO: 28 | ASTKGPSVFPLAPSSKSTSGGTAALGCLVKDYFPEPVTVSWNSGA | Human IgG1 Fc |
| | LTSGVHTFPAVLQSSGLYSLSSVVTVPSSSLGTQTYICNVNHKPS | domain (CH1-hinge- |
| | NTKVDKKVEPKSCDKTHTCPPCPAPELLGGPSVELFPPKPKDTLM | CH2,CH3) |
| | ISRTPEVTCVVVDVSHEDPEVKENWYVDGVEVHNAKTKPREEQYN | |
| | STYRVVSVLTVLHQDWLNGKEYKCKVSNKALPAPIEKTISKAKGQ | |
| | PREPQVYTLPPSRDELTKNQVSLTCLVKGFYPSDIAVEWESNGQP | |
| | ENNYKTTPPVLDSDGSFFLYSKLTVDKSRWQQGNVFSCSVMHEAL | |
| | HNHYTQKSLSLSPGK | |
| SEQ ID NO: 29 | MDEKTTGWRGGHVVEGLAGELEQLRARLEHHPQGQREP | Streptavidin binding |
| | | peptide |
| SEQ ID NO: 30 | WSHPQFEK | Strep TagII |
| SEQ ID NO: 31 | WSHPQFEKGGGSGGGSGGSAWSHPQFEK | TwinStrepTag |
| SEQ ID NO: 32 | MFVFLVLLPLVSSQCVNLTTRTQLPPAYTNSFTRGVYYPDKVERS | Full length Spike |
| | SVLHSTQDLFLPFFSNVTWFHAIHVSGTNGTKRFDNPVLPENDGV | protein |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTFLLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLFRKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPSSASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGENESQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWP | |
| SEQ ID NO: 33 | SQCVNLTTRTQLPPAYTNSFTRGVYYPDKVFRSSVLHSTQDLFLP | S1 |
| | FFSNVTWFHAIHVSGTNGTKREDNPVLPENDGVYFASTEKSNIIR | |
| | GWIFGTTLDSKTQSLLIVNNATNVVIKVCEFQFCNDPFLGVYYHK | |
| | NNKSWMESEFRVYSSANNCTFEYVSQPFLMDLEGKQGNEKNLREF | |
| | VFKNIDGYFKIYSKHTPINLVRDLPQGESALEPLVDLPIGINITR | |
| | FQTLLALHRSYLTPGDSSSGWTAGAAAYYVGYLQPRTELLKYNEN | |
| | GTITDAVDCALDPLSETKCTLKSFTVEKGIYQTSNERVQPTESIV | |
| | RFPNITNLCPFGEVENATRFASVYAWNRKRISNCVADYSVLYNSA | |
| | SFSTFKCYGVSPTKLNDLCFTNVYADSEVIRGDEVRQIAPGQTGK | |
| | IADYNYKLPDDFTGCVIAWNSNNLDSKVGGNYNYLYRLERKSNLK | |
| | PFERDISTEIYQAGSTPCNGVEGENCYFPLQSYGFQPTNGVGYQP | |
| | YRVVVLSFELLHAPATVCGPKKSTNLVKNKCVNFNFNGLTGTGVL | |
| | TESNKKELPFQQFGRDIADTTDAVRDPQTLEILDITPCSFGGVSV | |
| | ITPGTNTSNQVAVLYQDVNCTEVPVAIHADQLTPTWRVYSTGSNV | |
| | FQTRAGCLIGAEHVNNSYECDIPIGAGICASYQTQTNSPSSAS | |
| SEQ ID NO: 34 | RVQPTESIVRFPNITNLCPFGEVENATRFASVYAWNRKRISNCVA | RBD |
| | DYSVLYNSASFSTFKCYGVSPTKLNDLCFTNVYADSFVIRGDEVR | |
| | QIAPGQTGKIADYNYKLPDDFTGCVIAWNSNNLDSKVGGNYNYLY | |
| | RLFRKSNLKPFERDISTEIYQAGSTPCNGVEGENCYFPLQSYGFQ | |
| | PTNGVGYQPYRVVVLSFELLHAPATVCGPKKSTNLVKNKCVNF | |
| SEQ ID NO: 35 | SVASQSIIAYTMSLGAENSVAYSNNSIAIPTNFTISVTTEILPVS | S2 |
| | MTKTSVDCTMYICGDSTECSNLLLQYGSFCTQLNRALTGIAVEQD | |
| | KNTQEVFAQVKQIYKTPPIKDFGGFNFSQILPDPSKPSKRSFIED | |
| | LLENKVTLADAGFIKQYGDCLGDIAARDLICAQKENGLTVLPPLL | |
| | TDEMIAQYTSALLAGTITSGWTFGAGAALQIPFAMQMAYRENGIG | |
| | VTQNVLYENQKLIANQFNSAIGKIQDSLSSTASALGKLQDVVNQN | |
| | AQALNTLVKQLSSNFGAISSVLNDILSRLDPPEAEVQIDRLITGR | |
| | LQSLQTYVTQQLIRAAEIRASANLAATKMSECVLGQSKRVDECGK | |
| | GYHLMSFPQSAPHGVVFLHVTYVPAQEKNFTTAPAICHDGKAHFP | |
| | REGVFVSNGTHWFVTQRNFYEPQIITTDNTFVSGNCDVVIGIVNN | |
| | TVYDPLQPELDSFKEELDKYFKNHTSPDVDLGDISGINASVVNIQ | |
| | KEIDRLNEVAKNLNESLIDLQELGKYEQYIKWP | |
| SEQ ID NO: 36 | VPEVSSVFIFPPKPKDVLTITLTPKVTCVVVDISKDDPEVQESWE | Murine IgG Fc |
| | VDDVEVHTAQTQPREEQFNSTFRSVSELPIMHQDWLNGKEFKCRV | |
| | NSAAFPAPIEKTISKTKGRPKAPQVYTIPPPKEQMAKDKVSLTCM | |
| | ITDFFPEDITVEWQWNGQPAENYKNTQPIMDTDGSYFVYSKLNVQ | |
| | KSNWEAGNTFTCSVLHEGLHNHHTEKSLSHSPGI | |
| SEQ ID NO: 37 | MFVFLVLLPLVSSQCVNFTTRNQLPSAYTNSFTRGVYYPDKVERS | pxENB9-FLS-P.1 |
| | SVLHSTQDLFLPFFSNVTWFHAIHVSGTNGTKRFDNPVLPENDGV | |
| | YFASTEKSNIIRGWIFGTTLDSKTQSLLIVNNATNVVIKVCEFQF | |
| | CNYPFLGVYYHKNNKSWMESEFRVYSSANNCTFEYVSQPELMDLE | |
| | GKQGNFKNLSEFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGTIADYNYKLPDDFTGCVIAWNSNNLDSKVGGNYN | |
| | YLYRLERKSNLKPFERDISTEIYQAGSTPCNGVKGENCYFPLQSY | |
| | GFQPTYGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQGVNCTEVPVAIHADQLT | |
| | PTWRVYSTGSNVFQTRAGCLIGAEYVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQTYKTPPIKDFGGENFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAAIKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSGYIPEAPRDGQAYVRKDGEWVLLSTFLGGGSHHHHHHHH | |
| SEQ ID NO: 38 | MSSSSWLLLSLVAVTAAQSTIEEQAKTELDKENHEAEDLFYQSSL | ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DNPQECLLLEPGLNEIMANSLDYNERLWAWESWRSEVGKQLRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPELLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLENMLRLGKSEPWTLALENVVGAKNMNVRPLL | |
| | NYFEPLFTWLKDQNKNSFVGWSTDWSPYAD | |
| SEQ ID NO: 39 | MSDNGPQNQRNAPRITFGGPSDSTGSNQNGERSGARSKQRRPQGL | SARS-CoV-2 |
| | PNNTASWFTALTQHGKEDLKFPRGQGVPINTNSSPDDQIGYYRRA | nucleocapsid N- |
| | TRRIRGGDGKMKDLSPRWYFYYLGTGPEAGLPYGANKDGIIWVAT | terminal domain |
| | EGALNTPKDHIGTRNPANNAAIVLQLPQGTTLPKGFYAEGSRGGS | |
| | QA | |
| SEQ ID NO: 40 | MFVFLVLLPLVSSQCVNLTTRTQLPPAYTNSFTRGVYYPDKVERS | pxAM-FLS-A |
| | SVLHSTQDLFLPFFSNVTWFHAIHVSGTNGTKRFDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTFLLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLERKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPASVASQSIIAYTMSLGAENSVAYSNNSIAIPTNFTISVT | |
| | TEILPVSMTKTSVDCTMYICGDSTECSNLLLQYGSFCTQLNRALT | |
| | GIAVEQDKNTQEVFAQVKQIYKTPPIKDEGGENESQILPDPSKPS | |
| | KRSFIEDLLENKVTLADAGFIKQYGDCLGDIAARDLICAQKENGL | |
| | TVLPPLLTDEMIAQYTSALLAGTITSGWTFGAGAALQIPFAMQMA | |
| | YRENGIGVTQNVLYENQKLIANQFNSAIGKIQDSLSSTASALGKL | |
| | QDVVNQNAQALNTLVKQLSSNFGAISSVLNDILSRLDPPEAEVQI | |
| | DRLITGRLQSLQTYVTQQLIRAAEIRASANLAATKMSECVLGQSK | |
| | RVDFCGKGYHLMSFPQSAPHGVVFLHVTYVPAQEKNFTTAPAICH | |
| | DGKAHFPREGVFVSNGTHWFVTQRNFYEPIITTDNTFVSGNCDVV | |
| | IGIVNNTVYDPLQPELDSFKEELDKYFKNHTSPDVDLGDISGINA | |
| | SVVNIQKEIDRLNEVAKNLNESLIDLQELGKYEQYIKWPLVPRGS | |
| | GSGYIPEAPRDGQAYVRKDGEWVLLSTFLGHHHHHH | |
| SEQ ID NO: 41 | MFVFLVLLPLVSSQCVNLTTRTQLPPAYTNSFTRGVYYPDKVERS | pxAM-FLS-SSAS |
| | SVLHSTQDLFLPFFSNVTWFHAIHVSGTNGTKREDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNERVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLFRKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPSSASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGFNFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPLV | |
| | PRGSGSGYIPEAPRDGQAYVRKDGEWVLLSTELGHHHHHH | |
| SEQ ID NO: 42 | MFVFLVLLPLVSSQHHHHHHHHGGGSENLYFQCVNLTTRTQLPPA | pxENB1-FLS |
| | YTNSFTRGVYYPDKVERSSVLHSTQDLFLPFFSNVTWFHAIHVSG | |
| | TNGTKRFDNPVLPENDGVYFASTEKSNIIRGWIFGTTLDSKTQSL | |
| | LIVNNATNVVIKVCEFQFCNDPFLGVYYHKNNKSWMESEFRVYSS | |
| | ANNCTFEYVSQPFLMDLEGKQGNEKNLREFVEKNIDGYFKIYSKH | |
| | TPINLVRDLPQGFSALEPLVDLPIGINITRFQTLLALHRSYLTPG | |
| | DSSSGWTAGAAAYYVGYLQPRTELLKYNENGTITDAVDCALDPLS | |
| | ETKCTLKSFTVEKGIYQTSNERVQPTESIVRFPNITNLCPFGEVE | |
| | NATRFASVYAWNRKRISNCVADYSVLYNSASESTFKCYGVSPTKL | |
| | NDLCFTNVYADSFVIRGDEVRQIAPGQTGKIADYNYKLPDDETGC | |
| | VIAWNSNNLDSKVGGNYNYLYRLFRKSNLKPFERDISTEIYQAGS | |
| | TPCNGVEGENCYFPLQSYGFQPTNGVGYQPYRVVVLSFELLHAPA | |
| | TVCGPKKSTNLVKNKCVNFNFNGLTGTGVLTESNKKELPFQQFGR | |
| | DIADTTDAVRDPQTLEILDITPCSFGGVSVITPGTNTSNQVAVLY | |
| | QDVNCTEVPVAIHADQLTPTWRVYSTGSNVFQTRAGCLIGAEHVN | |
| | NSYECDIPIGAGICASYQTQTNSPGAASSVASQSIIAYTMSLGAE | |
| | NSVAYSNNSIAIPTNFTISVTTEILPVSMTKTSVDCTMYICGDST | |
| | ECSNLLLQYGSFCTQLNRALTGIAVEQDKNTQEVFAQVKQIYKTP | |
| | PIKDFGGFNFSQILPDPSKPSKRSFIEDLLENKVTLADAGFIKQY | |
| | GDCLGDIAARDLICAQKENGLTVLPPLLTDEMIAQYTSALLAGTI | |
| | TSGWTFGAGAALQIPFAMQMAYRENGIGVTQNVLYENQKLIANQE | |
| | NSAIGKIQDSLSSTASALGKLQDVVNQNAQALNTLVKQLSSNEGA | |
| | ISSVLNDILSRLDPPEAEVQIDRLITGRLQSLQTYVTQQLIRAAE | |
| | IRASANLAATKMSECVLGQSKRVDFCGKGYHLMSFPQSAPHGVVF | |
| | LHVTYVPAQEKNFTTAPAICHDGKAHFPREGVFVSNGTHWFVTQR | |
| | NFYEPQIITTDNTFVSGNCDVVIGIVNNTVYDPLQPELDSFKEEL | |
| | DKYFKNHTSPDVDLGDISGINASVVNIQKEIDRLNEVAKNLNESL | |
| | IDLQELGKYEQYIKWPGGGSLKSRLDTLSQEVALLKEQQALQTVC | |
| | L | |
| SEQ ID NO: 43 | MFVFLVLLPLVSSQHHHHHHHHGGGSENLYFQCVNLTTRTQLPPA | pxENB2-FLS |
| | YTNSFTRGVYYPDKVFRSSVLHSTQDLFLPFFSNVTWFHAIHVSG | |
| | TNGTKRFDNPVLPENDGVYFASTEKSNIIRGWIFGTTLDSKTQSL | |
| | LIVNNATNVVIKVCEFQFCNDPFLGVYYHKNNKSWMESEFRVYSS | |
| | ANNCTFEYVSQPFLMDLEGKQGNEKNLREFVEKNIDGYFKIYSKH | |
| | TPINLVRDLPQGFSALEPLVDLPIGINITRFQTLLALHRSYLTPG | |
| | DSSSGWTAGAAAYYVGYLQPRTELLKYNENGTITDAVDCALDPLS | |
| | ETKCTLKSFTVEKGIYQTSNERVQPTESIVRFPNITNLCPFGEVE | |
| | NATRFASVYAWNRKRISNCVADYSVLYNSASFSTFKCYGVSPTKL | |
| | NDLCFTNVYADSFVIRGDEVRQIAPGQTGKIADYNYKLPDDETGC | |
| | VIAWNSNNLDSKVGGNYNYLYRLERKSNLKPFERDISTEIYQAGS | |
| | TPCNGVEGENCYFPLQSYGFQPTNGVGYQPYRVVVLSFELLHAPA | |
| | TVCGPKKSTNLVKNKCVNFNFNGLTGTGVLTESNKKELPFQQFGR | |
| | DIADTTDAVRDPQTLEILDITPCSFGGVSVITPGTNTSNQVAVLY | |
| | QDVNCTEVPVAIHADQLTPTWRVYSTGSNVFQTRAGCLIGAEHVN | |
| | NSYECDIPIGAGICASYQTQTNSPGAASSVASQSIIAYTMSLGAE | |
| | NSVAYSNNSIAIPTNFTISVTTEILPVSMTKTSVDCTMYICGDST | |
| | ECSNLLLQYGSFCTQLNRALTGIAVEQDKNTQEVFAQVKQIYKTP | |
| | PIKDFGGFNFSQILPDPSKPSKRSFIEDLLENKVTLADAGFIKQY | |
| | GDCLGDIAARDLICAQKENGLTVLPPLLTDEMIAQYTSALLAGTI | |
| | TSGWTFGAGAALQIPFAMQMAYRENGIGVTQNVLYENQKLIANQE | |
| | NSAIGKIQDSLSSTASALGKLQDVVNQNAQALNTLVKQLSSNFGA | |
| | ISSVLNDILSRLDPPEAEVQIDRLITGRLQSLQTYVTQQLIRAAE | |
| | IRASANLAATKMSECVLGQSKRVDFCGKGYHLMSFPQSAPHGVVE | |
| | LHVTYVPAQEKNFTTAPAICHDGKAHFPREGVFVSNGTHWFVTQR | |
| | NFYEPQIITTDNTFVSGNCDVVIGIVNNTVYDPLQPELDSFKEEL | |
| | DKYFKNHTSPDVDLGDISGINASVVNIQKEIDRLNEVAKNLNESL | |
| | IDLQELGKYEQYIKWPGGGSGYIPEAPRDGQAYVRKDGEWVLLST | |
| | FL | |
| SEQ ID NO: 44 | MFVFLVLLPLVSSQHHHHHHHHGGGSENLYFQCVNLTTRTQLPPA | pxENB5-FLS |
| | YTNSFTRGVYYPDKVFRSSVLHSTQDLFLPFFSNVTWFHAIHVSG | |
| | TNGTKRFDNPVLPENDGVYFASTEKSNIIRGWIFGTTLDSKTQSL | |
| | LIVNNATNVVIKVCEFQFCNDPFLGVYYHKNNKSWMESEFRVYSS | |
| | ANNCTFEYVSQPFLMDLEGKQGNEKNLREFVEKNIDGYFKIYSKH | |
| | TPINLVRDLPQGFSALEPLVDLPIGINITRFQTLLALHRSYLTPG | |
| | DSSSGWTAGAAAYYVGYLQPRTELLKYNENGTITDAVDCALDPLS | |
| | ETKCTLKSFTVEKGIYQTSNERVQPTESIVRFPNITNLCPFGEVE | |
| | NATRFASVYAWNRKRISNCVADYSVLYNSASFSTFKCYGVSPTKL | |
| | NDLCFTNVYADSFVIRGDEVRQIAPGQTGKIADYNYKLPDDFTGC | |
| | VIAWNSNNLDSKVGGNYNYLYRLERKSNLKPFERDISTEIYQAGS | |
| | TPCNGVEGENCYFPLQSYGFQPTNGVGYQPYRVVVLSFELLHAPA | |
| | TVCGPKKSTNLVKNKCVNFNFNGLTGTGVLTESNKKELPFQQFGR | |
| | DIADTTDAVRDPQTLEILDITPCSFGGVSVITPGTNTSNQVAVLY | |
| | QDVNCTEVPVAIHADQLTPTWRVYSTGSNVFQTRAGCLIGAEHVN | |
| | NSYECDIPIGAGICASYQTQTNSPGAASSVASQSIIAYTMSLGAE | |
| | NSVAYSNNSIAIPTNFTISVTTEILPVSMTKTSVDCTMYICGDST | |
| | ECSNLLLQYGSFCTQLNRALTGIAVEQDKNTQEVFAQVKQIYKTP | |
| | PIKDFGGFNFSQILPDPSKPSKRSFIEDLLENKVTLADAGFIKQY | |
| | GDCLGDIAARDLICAQKENGLTVLPPLLTDEMIAQYTSALLAGTI | |
| | TSGWTFGAGAALQIPFAMQMAYRENGIGVTQNVLYENQKLIANQF | |
| | NSAIGKIQDSLSSTASALGKLQDVVNQNAQALNTLVKQLSSNFGA | |
| | ISSVLNDILSRLDPPEAEVQIDRLITGRLQSLQTYVTQQLIRAAE | |
| | IRASANLAATKMSECVLGQSKRVDFCGKGYHLMSFPQSAPHGVVE | |
| | LHVTYVPAQEKNFTTAPAICHDGKAHFPREGVFVSNGTHWFVTQR | |
| | NFYEPQIITTDNTFVSGNCDVVIGIVNNTVYDPLQPELDSFKEEL | |
| | DKYFKNHTSPDVDLGDISGINASVVNIQKEIDRLNEVAKNLNESL | |
| | IDLQELGKYEQYIKWP | |
| SEQ ID NO: 45 | MFVFLVLLPLVSSQCVNLTTRTQLPPAYTNSFTRGVYYPDKVERS | pxENB8-FLS |
| | SVLHSTQDLFLPFFSNVTWFHAIHVSGTNGTKRFDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLERKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGFNFSQILPDPS | |
| | KPSKRSFIEDLLENKVTLADAGFIKQYGDCLGDIAARDLICAQKF | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSLKSRLDTLSQEVALLKEQQALQTVCLGGGSHHHHHHHH | |
| SEQ ID NO: 46 | MFVFLVLLPLVSSQCVNLTTRTQLPPAYTNSFTRGVYYPDKVERS | PxENB9-FLS |
| | SVLHSTQDLFLPFFSNVTWFHAIHVSGTNGTKREDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGFSALEP | |
| | LVDLPIGINITRFQTLLALHRSYLTPGDSSSGWTAGAAAYYVGYL | |
| | QPRTFLLKYNENGTITDAVDCALDPLSETKCTLKSFTVEKGIYQT | |
| | SNERVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDETGCVIAWNSNNLDSKVGGNYN | |
| | YLYRLFRKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGENFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSGYIPEAPRDGQAYVRKDGEWVLLSTFLGGGSHHHHHHHH | |
| SEQ ID NO: 47 | MFVFLVLLPLVSSQCVNLTTRTQLPPAYTNSFTRGVYYPDKVERS | PxENB12-FLS |
| | SVLHSTQDLFLPFFSNVTWFHAIHVSGTNGTKREDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNEKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTFLLKYNENGTITDAVDCALDPLSETKCTLKSFTVEKGIYQT | |
| | SNERVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLERKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGENFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSHHHHHHHH | |
| SEQ ID NO: 48 | MDAMKRGLCCVLLLCGAVFVSPSVNLTTRTQLPPAYTNSFTRGVY | PxENBCP14-FLS |
| | YPDKVFRSSVLHSTQDLFLPFFSNVTWFHAIHVSGTNGTKREDNP | |
| | VLPENDGVYFASTEKSNIIRGWIFGTTLDSKTQSLLIVNNATNVV | |
| | IKVCEFQFCNDPFLGVYYHKNNKSWMESEFRVYSSANNCTFEYVS | |
| | QPFLMDLEGKQGNEKNLREFVEKNIDGYFKIYSKHTPINLVRDLP | |
| | QGFSALEPLVDLPIGINITRFQTLLALHRSYLTPGDSSSGWTAGA | |
| | AAYYVGYLQPRTELLKYNENGTITDAVDCALDPLSETKCTLKSFT | |
| | VEKGIYQTSNERVQPTESIVRFPNITNLCPFGEVENATRFASVYA | |
| | WNRKRISNCVADYSVLYNSASFSTFKCYGVSPTKLNDLCFTNVYA | |
| | DSFVIRGDEVRQIAPGQTGKIADYNYKLPDDFTGCVIAWNSNNLD | |
| | SKVGGNYNYLYRLERKSNLKPFERDISTEIYQAGSTPCNGVEGEN | |
| | CYFPLQSYGFQPTNGVGYQPYRVVVLSFELLHAPATVCGPKKSTN | |
| | LVKNKCVNFNFNGLTGTGVLTESNKKELPFQQFGRDIADTTDAVR | |
| | DPQTLEILDITPCSFGGVSVITPGTNTSNQVAVLYQDVNCTEVPV | |
| | AIHADQLTPTWRVYSTGSNVFQTRAGCLIGAEHVNNSYECDIPIG | |
| | AGICASYQTQTNSPGAASSVASQSIIAYTMSLGAENSVAYSNNSI | |
| | AIPTNFTISVTTEILPVSMTKTSVDCTMYICGDSTECSNLLLQYG | |
| | SFCTQLNRALTGIAVEQDKNTQEVFAQVKQIYKTPPIKDEGGENF | |
| | SQILPDPSKPSKRSFIEDLLENKVTLADAGFIKQYGDCLGDIAAR | |
| | DLICAQKENGLTVLPPLLTDEMIAQYTSALLAGTITSGWTFGAGA | |
| | ALQIPFAMQMAYRENGIGVTQNVLYENQKLIANQFNSAIGKIQDS | |
| | LSSTASALGKLQDVVNQNAQALNTLVKQLSSNFGAISSVLNDILS | |
| | RLDPPEAEVQIDRLITGRLQSLQTYVTQQLIRAAEIRASANLAAT | |
| | KMSECVLGQSKRVDFCGKGYHLMSFPQSAPHGVVFLHVTYVPAQE | |
| | KNFTTAPAICHDGKAHFPREGVFVSNGTHWFVTQRNFYEPQIITT | |
| | DNTFVSGNCDVVIGIVNNTVYDPLQPELDSFKEELDKYFKNHTSP | |
| | DVDLGDISGINASVVNIQKEIDRLNEVAKNLNESLIDLQELGKYE | |
| | QYIKWPGGGSGYIPEAPRDGQAYVRKDGEWVLLSTFLGGGSHHHH | |
| | HHHH | |
| SEQ ID NO: 49 | MEWSWVFLFFLSVTTGVHSVNLTTRTQLPPAYTNSFTRGVYYPDK | PxENBCP15-FLS |
| | VFRSSVLHSTQDLFLPFFSNVTWFHAIHVSGTNGTKREDNPVLPF | |
| | NDGVYFASTEKSNIIRGWIFGTTLDSKTQSLLIVNNATNVVIKVC | |
| | EFQFCNDPFLGVYYHKNNKSWMESEFRVYSSANNCTFEYVSQPFL | |
| | MDLEGKQGNEKNLREFVEKNIDGYFKIYSKHTPINLVRDLPQGES | |
| | ALEPLVDLPIGINITRFQTLLALHRSYLTPGDSSSGWTAGAAAYY | |
| | VGYLQPRTELLKYNENGTITDAVDCALDPLSETKCTLKSFTVEKG | |
| | IYQTSNFRVQPTESIVRFPNITNLCPFGEVENATRFASVYAWNRK | |
| | RISNCVADYSVLYNSASFSTFKCYGVSPTKLNDLCFTNVYADSFV | |
| | IRGDEVRQIAPGQTGKIADYNYKLPDDFTGCVIAWNSNNLDSKVG | |
| | GNYNYLYRLERKSNLKPFERDISTEIYQAGSTPCNGVEGENCYFP | |
| | LQSYGFQPTNGVGYQPYRVVVLSFELLHAPATVCGPKKSTNLVKN | |
| | KCVNFNFNGLTGTGVLTESNKKELPFQQFGRDIADTTDAVRDPQT | |
| | LEILDITPCSFGGVSVITPGTNTSNQVAVLYQDVNCTEVPVAIHA | |
| | DQLTPTWRVYSTGSNVFQTRAGCLIGAEHVNNSYECDIPIGAGIC | |
| | ASYQTQTNSPGAASSVASQSIIAYTMSLGAENSVAYSNNSIAIPT | |
| | NFTISVTTEILPVSMTKTSVDCTMYICGDSTECSNLLLQYGSFCT | |
| | QLNRALTGIAVEQDKNTQEVFAQVKQIYKTPPIKDFGGENESQIL | |
| | PDPSKPSKRSFIEDLLENKVTLADAGFIKQYGDCLGDIAARDLIC | |
| | AQKFNGLTVLPPLLTDEMIAQYTSALLAGTITSGWTFGAGAALQI | |
| | PFAMQMAYRENGIGVTQNVLYENQKLIANQFNSAIGKIQDSLSST | |
| | ASALGKLQDVVNQNAQALNTLVKQLSSNFGAISSVLNDILSRLDP | |
| | PEAEVQIDRLITGRLQSLQTYVTQQLIRAAEIRASANLAATKMSE | |
| | CVLGQSKRVDFCGKGYHLMSFPQSAPHGVVELHVTYVPAQEKNET | |
| | TAPAICHDGKAHFPREGVFVSNGTHWFVTQRNFYEPQIITTDNTE | |
| | VSGNCDVVIGIVNNTVYDPLQPELDSFKEELDKYFKNHTSPDVDL | |
| | GDISGINASVVNIQKEIDRLNEVAKNLNESLIDLQELGKYEQYIK | |
| | WPGGGSGYIPEAPRDGQAYVRKDGEWVLLSTFLGGGSHHHHHHHH | |
| SEQ ID NO: 50 | MYRMQLLSCIALSLALVNLTTRTQLPPAYTNSFTRGVYYPDKVER | PxENBCP16-FLS |
| | SSVLHSTQDLFLPFFSNVTWFHAIHVSGTNGTKRFDNPVLPENDG | |
| | VYFASTEKSNIIRGWIFGTTLDSKTQSLLIVNNATNVVIKVCEFQ | |
| | FCNDPFLGVYYHKNNKSWMESEFRVYSSANNCTFEYVSQPFLMDL | |
| | EGKQGNFKNLREFVFKNIDGYFKIYSKHTPINLVRDLPQGESALE | |
| | PLVDLPIGINITRFQTLLALHRSYLTPGDSSSGWTAGAAAYYVGY | |
| | LQPRTFLLKYNENGTITDAVDCALDPLSETKCTLKSFTVEKGIYQ | |
| | TSNFRVQPTESIVRFPNITNLCPFGEVENATRFASVYAWNRKRIS | |
| | NCVADYSVLYNSASFSTFKCYGVSPTKLNDLCFTNVYADSFVIRG | |
| | DEVRQIAPGQTGKIADYNYKLPDDFTGCVIAWNSNNLDSKVGGNY | |
| | NYLYRLFRKSNLKPFERDISTEIYQAGSTPCNGVEGENCYFPLQS | |
| | YGFQPTNGVGYQPYRVVVLSFELLHAPATVCGPKKSTNLVKNKCV | |
| | NFNFNGLTGTGVLTESNKKELPFQQFGRDIADTTDAVRDPQTLEI | |
| | LDITPCSFGGVSVITPGTNTSNQVAVLYQDVNCTEVPVAIHADQL | |
| | TPTWRVYSTGSNVFQTRAGCLIGAEHVNNSYECDIPIGAGICASY | |
| | QTQTNSPGAASSVASQSIIAYTMSLGAENSVAYSNNSIAIPTNFT | |
| | ISVTTEILPVSMTKTSVDCTMYICGDSTECSNLLLQYGSFCTQLN | |
| | RALTGIAVEQDKNTQEVFAQVKQIYKTPPIKDEGGENFSQILPDP | |
| | SKPSKRSFIEDLLENKVTLADAGFIKQYGDCLGDIAARDLICAQK | |
| | FNGLTVLPPLLTDEMIAQYTSALLAGTITSGWTFGAGAALQIPFA | |
| | MQMAYRENGIGVTQNVLYENQKLIANQFNSAIGKIQDSLSSTASA | |
| | LGKLQDVVNQNAQALNTLVKQLSSNFGAISSVLNDILSRLDPPEA | |
| | EVQIDRLITGRLQSLQTYVTQQLIRAAEIRASANLAATKMSECVL | |
| | GQSKRVDFCGKGYHLMSFPQSAPHGVVFLHVTYVPAQEKNETTAP | |
| | AICHDGKAHFPREGVFVSNGTHWFVTQRNFYEPQIITTDNTFVSG | |
| | NCDVVIGIVNNTVYDPLQPELDSFKEELDKYFKNHTSPDVDLGDI | |
| | SGINASVVNIQKEIDRLNEVAKNLNESLIDLQELGKYEQYIKWPG | |
| | GGSGYIPEAPRDGQAYVRKDGEWVLLSTFLGGGSHHHHHHHH | |
| SEQ ID NO: 51 | MFVFLVLLPLVSSQCVNLTTRTQLPPAYTNSFTRGVYYPDKVERS | PxENBCP17-FLS |
| | SVLHSTQDLFLPFFSNVTWFHAIHVSGTNGTKRFDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGFSALEP | |
| | LVDLPIGINITRFQTLLALHRSYLTPGDSSSGWTAGAAAYYVGYL | |
| | QPRTFLLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLERKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGENFSQILPDPS | |
| | KPSKRSFIEDLLENKVTLADAGFIKQYGDCLGDIAARDLICAQKF | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSGYIPEAPRDGQAYVRKDGEWVLLSTFLGGGSHHHHHHHHGGSG | |
| | MDEKTTGWRGGHVVEGLAGELEQLRARLEHHPQGQREP | |
| SEQ ID NO: 52 | MFVFLVLLPLVSSQCVNLTTRTQLPPAYTNSFTRGVYYPDKVERS | PxENBCP18-FLS |
| | SVLHSTQDLFLPFFSNVTWFHAIHVSGTNGTKREDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNERVQPTESIVREPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLERKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGFNFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVELHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSGYIPEAPRDGQAYVRKDGEWVLLSTEL | |
| SEQ ID NO: 53 | MFVFLVLLPLVSSQCVNLTTRTQLPPAYTNSFTRGVYYPDKVERS | PxENBCP19-FLS |
| | SVLHSTQDLFLPFFSNVTWFHAIHVSGTNGTKRFDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGFSALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLERKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGFNFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSGYIPEAPRDGQAYVRKDGEWVLLSTFLGGGSHHHHHHHHGGSG | |
| | WSHPQFEK | |
| SEQ ID NO: 54 | MFVFLVLLPLVSSQCVNLTTRTQLPPAYTNSFTRGVYYPDKVERS | PxENBCP20-FLS |
| | SVLHSTQDLFLPFFSNVTWFHAIHVSGTNGTKREDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLFRKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGENFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNETTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSGYIPEAPRDGQAYVRKDGEWVLLSTELGGGSHHHHHHHHGGSG | |
| | WSHPQFEKGGGSGGGSGGSAWSHPQFEK | |
| SEQ ID NO: 55 | MDAMKRGLCCVLLLCGAVFVSPSMSDNGPQNQRNAPRITFGGPSD | PxENBEP17-FLS |
| | STGSNQNGERSGARSKQRRPQGLPNNTASWFTALTQHGKEDLKEP | |
| | RGQGVPINTNSSPDDQIGYYRRATRRIRGGDGKMKDLSPRWYFYY | |
| | LGTGPEAGLPYGANKDGIIWVATEGALNTPKDHIGTRNPANNAAI | |
| | VLQLPQGTTLPKGFYAEGSRGGSQAGGSGGCVNLTTRTQLPPAYT | |
| | NSFTRGVYYPDKVERSSVLHSTQDLFLPFFSNVTWFHAIHVSGTN | |
| | GTKRFDNPVLPENDGVYFASTEKSNIIRGWIFGTTLDSKTQSLLI | |
| | VNNATNVVIKVCEFQFCNDPFLGVYYHKNNKSWMESEFRVYSSAN | |
| | NCTFEYVSQPFLMDLEGKQGNEKNLREFVEKNIDGYFKIYSKHTP | |
| | INLVRDLPQGFSALEPLVDLPIGINITRFQTLLALHRSYLTPGDS | |
| | SSGWTAGAAAYYVGYLQPRTELLKYNENGTITDAVDCALDPLSET | |
| | KCTLKSFTVEKGIYQTSNERVQPTESIVRFPNITNLCPFGEVENA | |
| | TREASVYAWNRKRISNCVADYSVLYNSASFSTFKCYGVSPTKLND | |
| | LCFTNVYADSFVIRGDEVRQIAPGQTGKIADYNYKLPDDFTGCVI | |
| | AWNSNNLDSKVGGNYNYLYRLFRKSNLKPFERDISTEIYQAGSTP | |
| | CNGVEGENCYFPLQSYGFQPTNGVGYQPYRVVVLSFELLHAPATV | |
| | CGPKKSTNLVKNKCVNFNFNGLTGTGVLTESNKKELPFQQFGRDI | |
| | ADTTDAVRDPQTLEILDITPCSFGGVSVITPGTNTSNQVAVLYQD | |
| | VNCTEVPVAIHADQLTPTWRVYSTGSNVFQTRAGCLIGAEHVNNS | |
| | YECDIPIGAGICASYQTQTNSPGAASSVASQSIIAYTMSLGAENS | |
| | VAYSNNSIAIPTNFTISVTTEILPVSMTKTSVDCTMYICGDSTEC | |
| | SNLLLQYGSFCTQLNRALTGIAVEQDKNTQEVFAQVKQIYKTPPI | |
| | KDFGGFNFSQILPDPSKPSKRSFIEDLLENKVTLADAGFIKQYGD | |
| | CLGDIAARDLICAQKENGLTVLPPLLTDEMIAQYTSALLAGTITS | |
| | GWTFGAGAALQIPFAMQMAYRENGIGVTQNVLYENQKLIANQENS | |
| | AIGKIQDSLSSTASALGKLQDVVNQNAQALNTLVKQLSSNFGAIS | |
| | SVLNDILSRLDPPEAEVQIDRLITGRLQSLQTYVTQQLIRAAEIR | |
| | ASANLAATKMSECVLGQSKRVDECGKGYHLMSFPQSAPHGVVELH | |
| | VTYVPAQEKNFTTAPAICHDGKAHFPREGVFVSNGTHWFVTQRNE | |
| | YEPQIITTDNTFVSGNCDVVIGIVNNTVYDPLQPELDSFKEELDK | |
| | YFKNHTSPDVDLGDISGINASVVNIQKEIDRLNEVAKNLNESLID | |
| | LQELGKYEQYIKWPgggsHHHHHHHHHH | |
| SEQ ID NO: 56 | MFVFLVLLPLVSSQCVNLTTRTQLPPAYTNSFTRGVYYPDKVERS | PxENBEP18-FLS |
| | SVLHSTQDLFLPFFSNVTWFHAIHVSGTNGTKRFDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNFRVQPTESIVREPNITNLCPFGEVENATRFASVYAWNRKRISN | |
| | CVADYSVLYNSASFSTFKCYGVSPTKLNDLCFTNVYADSEVIRGD | |
| | EVRQIAPGQTGKIADYNYKLPDDFTGCVIAWNSNNLDSKVGGNYN | |
| | YLYRLFRKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | SVTTEILPVSMTKTSVDCTMYICGDSTECSNLLLQYGSFCTQLNR | |
| | TQTNSPGAASSVASQSIIAYTMSLGAENSVAYSNNSIAIPTNFTI | |
| | ALTGIAVEQDKNTQEVFAQVKQIYKTPPIKDFGGENFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | SGGMSDNGPQNQRNAPRITFGGPSDSTGSNQNGERSGARSKQRRP | |
| | QGLPNNTASWFTALTQHGKEDLKFPRGQGVPINTNSSPDDQIGYY | |
| | RRATRRIRGGDGKMKDLSPRWYFYYLGTGPEAGLPYGANKDGIIW | |
| | VATEGALNTPKDHIGTRNPANNAAIVLQLPQGTTLPKGFYAEGSR | |
| | GGSQAGGGSHHHHHHHHHH | |
| SEQ ID NO: 57 | MFVFLVLLPLVSSQCVNLTTRTQLPPAYTNSFTRGVYYPDKVERS | PxENBEP21-FLS |
| | SVLHSTQDLFLPFFSNVTWFHAIHVSGTNGTKREDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLERKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGFNFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSWKQSVELAKKDSLYKDAMQYASESKDTELAEELLQWELQEEKR | |
| | ECFGACLFTCYDLLRPDVVLELAWRHNIMDFAMPYFIQVMKEYLT | |
| | KVDKLDASESLRKGGGSHHHHHHHH | |
| SEQ ID NO: 58 | MFVFLVLLPLVSSQCVNLTTRTQLPPAYTNSFTRGVYYPDKVERS | PxENBEP22-FLS |
| | SVLHSTQDLFLPFFSNVTWFHAIHVSGTNGTKREDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLFRKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGENFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSRMKQLEDKVEELLSKNYHLENEVARLKKLVGERGGGSHHHHHH | |
| | HH | |
| SEQ ID NO: 59 | MFVFLVLLPLVSSQCVNLTTRTQLPPAYTNSFTRGVYYPDKVERS | PxENBEP23-FLS |
| | SVLHSTQDLFLPFFSNVTWFHAIHVSGTNGTKREDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPELMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDETGCVIAWNSNNLDSKVGGNYN | |
| | YLYRLERKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKFLPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGFNFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSASSGVRLWATRQAMLGQVHEVPEGWLIFVAEQEELYVRVQNGE | |
| | RKVQLEARTPLPRGGGSHHHHHHHH | |
| SEQ ID NO: 60 | MFVFLVLLPLVSSQCVNLTTRTQLPPAYTNSFTRGVYYPDKVERS | pxENBCP24-FLS |
| | SVLHSTQDLFLPFFSNVTWFHAIHVSGTNGTKRFDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNFRVQPTESIVREPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLFRKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGFNFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVELHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSWKQSVELAKKDSLYKDAMQYASESKDTELAEELLQWELQEEKR | |
| | ECFGACLFTCYDLLRPDVVLELAWRHNIMDFAMPYFIQVMKEYLT | |
| | KVDKLDASESLRKGGGSHHHHHHHHGGSGMDEKTTGWRGGHVVEG | |
| | LAGELEQLRARLEHHPQGQREP | |
| SEQ ID NO: 61 | MFVFLVLLPLVSSQCVNLTTRTQLPPAYTNSFTRGVYYPDKVERS | pxENBCP25-FLS |
| | SVLHSTQDLFLPFFSNVTWFHAIHVSGTNGTKRFDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLFRKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGENFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSRMKQLEDKVEELLSKNYHLENEVARLKKLVGERGGGSHHHHHH | |
| | HHGGSGMDEKTTGWRGGHVVEGLAGELEQLRARLEHHPQGQREP | |
| SEQ ID NO: 62 | MFVFLVLLPLVSSQCVNLTTRTQLPPAYTNSFTRGVYYPDKVERS | pxENBCP26-FLS |
| | SVLHSTQDLFLPFFSNVTWFHAIHVSGTNGTKREDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTFLLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLERKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGENFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQENSAIGKIQDSLSSTASAL | |
| | GKLQDVVNQNAQALNTLVKQLSSNEGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNETTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSASSGVRLWATRQAMLGQVHEVPEGWLIFVAEQEELYVRVQNGE | |
| | RKVQLEARTPLPRGGGSHHHHHHHHGGSGMDEKTTGWRGGHVVEG | |
| | LAGELEQLRARLEHHPQGQREP | |
| SEQ ID NO: 63 | MFVFLVLLPLVSSQCVNLTTRTQLPPAYTNSFTRGVYYPDKVERS | pO1-S1 |
| | SVLHSTQDLFLPFFSNVTWFHAIHVSGTNGTKRFDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTFLLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSEVIRGD | |
| | EVRQIAPGQTGKIADYNYKLPDDFTGCVIAWNSNNLDSKVGGNYN | |
| | YLYRLFRKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPHHHHHH | |
| SEQ ID NO: 64 | MYRMQLLSCIALSLALVNLTTRTQLPPAYTNSFTRGVYYPDKVER | pO2-S1 |
| | SSVLHSTQDLFLPFFSNVTWFHAIHVSGTNGTKRFDNPVLPENDG | |
| | VYFASTEKSNIIRGWIFGTTLDSKTQSLLIVNNATNVVIKVCEFQ | |
| | FCNDPFLGVYYHKNNKSWMESEFRVYSSANNCTFEYVSQPFLMDL | |
| | EGKQGNFKNLREFVEKNIDGYFKIYSKHTPINLVRDLPQGESALE | |
| | PLVDLPIGINITRFQTLLALHRSYLTPGDSSSGWTAGAAAYYVGY | |
| | LQPRTFLLKYNENGTITDAVDCALDPLSETKCTLKSFTVEKGIYQ | |
| | TSNERVQPTESIVRFPNITNLCPFGEVENATRFASVYAWNRKRIS | |
| | NCVADYSVLYNSASFSTFKCYGVSPTKLNDLCFTNVYADSFVIRG | |
| | DEVRQIAPGQTGKIADYNYKLPDDFTGCVIAWNSNNLDSKVGGNY | |
| | NYLYRLFRKSNLKPFERDISTEIYQAGSTPCNGVEGENCYFPLQS | |
| | YGFQPTNGVGYQPYRVVVLSFELLHAPATVCGPKKSTNLVKNKCV | |
| | NFNFNGLTGTGVLTESNKKELPFQQFGRDIADTTDAVRDPQTLEI | |
| | LDITPCSFGGVSVITPGTNTSNQVAVLYQDVNCTEVPVAIHADQL | |
| | TPTWRVYSTGSNVFQTRAGCLIGAEHVNNSYECDIPIGAGICASY | |
| | QTQTNSPHHHHHH | |
| SEQ ID NO: 65 | MDAMKRGLCCVLLLCGAVFVSPSVTNSVNLTTRTQLPPAYTNSFT | pO3-S1 |
| | RGVYYPDKVFRSSVLHSTQDLFLPFFSNVTWFHAIHVSGTNGTKR | |
| | FDNPVLPENDGVYFASTEKSNIIRGWIFGTTLDSKTQSLLIVNNA | |
| | TNVVIKVCEFQFCNDPFLGVYYHKNNKSWMESEFRVYSSANNCTE | |
| | EYVSQPFLMDLEGKQGNEKNLREFVEKNIDGYFKIYSKHTPINLV | |
| | RDLPQGFSALEPLVDLPIGINITRFQTLLALHRSYLTPGDSSSGW | |
| | TAGAAAYYVGYLQPRTELLKYNENGTITDAVDCALDPLSETKCTL | |
| | KSFTVEKGIYQTSNERVQPTESIVRFPNITNLCPFGEVENATRFA | |
| | SVYAWNRKRISNCVADYSVLYNSASFSTFKCYGVSPTKLNDLCFT | |
| | NVYADSFVIRGDEVRQIAPGQTGKIADYNYKLPDDFTGCVIAWNS | |
| | NNLDSKVGGNYNYLYRLERKSNLKPFERDISTEIYQAGSTPCNGV | |
| | EGENCYFPLQSYGFQPTNGVGYQPYRVVVLSFELLHAPATVCGPK | |
| | KSTNLVKNKCVNFNFNGLTGTGVLTESNKKELPFQQFGRDIADTT | |
| | DAVRDPQTLEILDITPCSFGGVSVITPGTNTSNQVAVLYQDVNCT | |
| | EVPVAIHADQLTPTWRVYSTGSNVFQTRAGCLIGAEHVNNSYECD | |
| | IPIGAGICASYQTQTNSPHHHHHH | |
| SEQ ID NO: 66 | MEWSWVFLFFLSVTTGVHSVTNSVNLTTRTQLPPAYTNSFTRGVY | pO4-S1 |
| | YPDKVFRSSVLHSTQDLFLPFFSNVTWFHAIHVSGTNGTKREDNP | |
| | VLPENDGVYFASTEKSNIIRGWIFGTTLDSKTQSLLIVNNATNVV | |
| | IKVCEFQFCNDPFLGVYYHKNNKSWMESEFRVYSSANNCTFEYVS | |
| | QPFLMDLEGKQGNEKNLREFVEKNIDGYFKIYSKHTPINLVRDLP | |
| | QGFSALEPLVDLPIGINITRFQTLLALHRSYLTPGDSSSGWTAGA | |
| | AAYYVGYLQPRTELLKYNENGTITDAVDCALDPLSETKCTLKSFT | |
| | VEKGIYQTSNERVQPTESIVRFPNITNLCPFGEVENATRFASVYA | |
| | WNRKRISNCVADYSVLYNSASFSTFKCYGVSPTKLNDLCFTNVYA | |
| | DSFVIRGDEVRQIAPGQTGKIADYNYKLPDDFTGCVIAWNSNNLD | |
| | SKVGGNYNYLYRLERKSNLKPFERDISTEIYQAGSTPCNGVEGEN | |
| | CYFPLQSYGFQPTNGVGYQPYRVVVLSFELLHAPATVCGPKKSTN | |
| | LVKNKCVNFNFNGLTGTGVLTESNKKELPFQQFGRDIADTTDAVR | |
| | DPQTLEILDITPCSFGGVSVITPGTNTSNQVAVLYQDVNCTEVPV | |
| | AIHADQLTPTWRVYSTGSNVFQTRAGCLIGAEHVNNSYECDIPIG | |
| | AGICASYQTQTNSPHHHHHH | |
| SEQ ID NO: 67 | MFVFLVLLPLVSSQHHHHHHHHGGGSENLYFQCVNLTTRTQLPPA | PxEBNCP1-S1 |
| | YTNSFTRGVYYPDKVERSSVLHSTQDLFLPFFSNVTWFHAIHVSG | |
| | TNGTKRFDNPVLPENDGVYFASTEKSNIIRGWIFGTTLDSKTQSL | |
| | LIVNNATNVVIKVCEFQFCNDPFLGVYYHKNNKSWMESEFRVYSS | |
| | ANNCTFEYVSQPFLMDLEGKQGNEKNLREFVEKNIDGYFKIYSKH | |
| | TPINLVRDLPQGFSALEPLVDLPIGINITRFQTLLALHRSYLTPG | |
| | DSSSGWTAGAAAYYVGYLQPRTELLKYNENGTITDAVDCALDPLS | |
| | ETKCTLKSFTVEKGIYQTSNERVQPTESIVRFPNITNLCPFGEVE | |
| | NATRFASVYAWNRKRISNCVADYSVLYNSASFSTFKCYGVSPTKL | |
| | NDLCFTNVYADSFVIRGDEVRQIAPGQTGKIADYNYKLPDDETGC | |
| | VIAWNSNNLDSKVGGNYNYLYRLERKSNLKPFERDISTEIYQAGS | |
| | TPCNGVEGENCYFPLQSYGFQPTNGVGYQPYRVVVLSFELLHAPA | |
| | TVCGPKKSTNLVKNKCVNFNFNGLTGTGVLTESNKKELPFQQFGR | |
| | DIADTTDAVRDPQTLEILDITPCSFGGVSVITPGTNTSNQVAVLY | |
| | QDVNCTEVPVAIHADQLTPTWRVYSTGSNVFQTRAGCLIGAEHVN | |
| | NSYECDIPIGAGICASYQTQTNS | |
| SEQ ID NO: 68 | MDAMKRGLCCVLLLCGAVEVSPSHHHHHHHHGGGSENLYFQCVNL | PxEBNCP2-S1 |
| | TTRTQLPPAYTNSFTRGVYYPDKVERSSVLHSTQDLFLPFFSNVT | |
| | WFHAIHVSGTNGTKRFDNPVLPENDGVYFASTEKSNIIRGWIFGT | |
| | TLDSKTQSLLIVNNATNVVIKVCEFQFCNDPFLGVYYHKNNKSWM | |
| | ESEFRVYSSANNCTFEYVSQPFLMDLEGKQGNEKNLREFVEKNID | |
| | GYFKIYSKHTPINLVRDLPQGESALEPLVDLPIGINITRFQTLLA | |
| | LHRSYLTPGDSSSGWTAGAAAYYVGYLQPRTELLKYNENGTITDA | |
| | VDCALDPLSETKCTLKSFTVEKGIYQTSNERVQPTESIVREPNIT | |
| | NLCPFGEVENATRFASVYAWNRKRISNCVADYSVLYNSASFSTEK | |
| | CYGVSPTKLNDLCFTNVYADSFVIRGDEVRQIAPGQTGKIADYNY | |
| | KLPDDFTGCVIAWNSNNLDSKVGGNYNYLYRLERKSNLKPFERDI | |
| | STEIYQAGSTPCNGVEGENCYFPLQSYGFQPTNGVGYQPYRVVVL | |
| | SFELLHAPATVCGPKKSTNLVKNKCVNENENGLTGTGVLTESNKK | |
| | FLPFQQFGRDIADTTDAVRDPQTLEILDITPCSFGGVSVITPGTN | |
| | TSNQVAVLYQDVNCTEVPVAIHADQLTPTWRVYSTGSNVFQTRAG | |
| | CLIGAEHVNNSYECDIPIGAGICASYQTQTNS | |
| SEQ ID NO: 69 | MFVFLVLLPLVSSQCVNLTTRTQLPPAYTNSFTRGVYYPDKVERS | PxEBNCP3-S1 |
| | SVLHSTQDLFLPFFSNVTWFHAIHVSGTNGTKREDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTFLLKYNENGTITDAVDCALDPLSETKCTLKSFTVEKGIYQT | |
| | SNFRVQPTESIVREPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLERKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGGGSHHHHHHHH | |
| SEQ ID NO: 70 | MDAMKRGLCCVLLLCGAVFVSPSCVNLTTRTQLPPAYTNSFTRGV | PxEBNCP4-S1 |
| | YYPDKVFRSSVLHSTQDLFLPFFSNVTWFHAIHVSGTNGTKREDN | |
| | PVLPENDGVYFASTEKSNIIRGWIFGTTLDSKTQSLLIVNNATNV | |
| | VIKVCEFQFCNDPFLGVYYHKNNKSWMESEFRVYSSANNCTFEYV | |
| | SQPFLMDLEGKQGNEKNLREFVFKNIDGYFKIYSKHTPINLVRDL | |
| | PQGFSALEPLVDLPIGINITRFQTLLALHRSYLTPGDSSSGWTAG | |
| | AAAYYVGYLQPRTELLKYNENGTITDAVDCALDPLSETKCTLKSF | |
| | TVEKGIYQTSNFRVQPTESIVRFPNITNLCPFGEVENATRFASVY | |
| | AWNRKRISNCVADYSVLYNSASFSTFKCYGVSPTKLNDLCFTNVY | |
| | ADSFVIRGDEVRQIAPGQTGKIADYNYKLPDDFTGCVIAWNSNNL | |
| | DSKVGGNYNYLYRLERKSNLKPFERDISTEIYQAGSTPCNGVEGF | |
| | NCYFPLQSYGFQPTNGVGYQPYRVVVLSFELLHAPATVCGPKKST | |
| | NLVKNKCVNFNFNGLTGTGVLTESNKKELPFQQFGRDIADTTDAV | |
| | RDPQTLEILDITPCSFGGVSVITPGTNTSNQVAVLYQDVNCTEVP | |
| | VAIHADQLTPTWRVYSTGSNVFQTRAGCLIGAEHVNNSYECDIPI | |
| | GAGICASYQTQTNSPGGGSHHHHHHHH | |
| SEQ ID NO: 71 | MFVFLVLLPLVSSQCVNLTTRTQLPPAYTNSFTRGVYYPDKVERS | PxEBNCP5-S1 |
| | SVLHSTQDLFLPFFSNVTWFHAIHVSGTNGTKREDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTFLLKYNENGTITDAVDCALDPLSETKCTLKSFTVEKGIYQT | |
| | SNFRVQPTESIVREPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLERKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPVPRDCGCKPCICTVPEVSSVFIFPPKPKDVLTITLTPKV | |
| | TCVVVDISKDDPEVQFSWFVDDVEVHTAQTQPREEQENSTERSVS | |
| | ELPIMHQDWLNGKEFKCRVNSAAFPAPIEKTISKTKGRPKAPQVY | |
| | TIPPPKEQMAKDKVSLTCMITDFFPEDITVEWQWNGQPAENYKNT | |
| | QPIMDTDGSYFVYSKLNVQKSNWEAGNTFTCSVLHEGLHNHHTEK | |
| | SLSHSPGI | |
| SEQ ID NO: 72 | MFVFLVLLPLVSSQCVNLTTRTQLPPAYTNSFTRGVYYPDKVERS | PxEBNCP7-S1 |
| | SVLHSTQDLFLPFFSNVTWFHAIHVSGTNGTKRFDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTFLLKYNENGTITDAVDCALDPLSETKCTLKSFTVEKGIYQT | |
| | SNFRVQPTESIVREPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLERKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGGGPVPEVSSVFIFPPKPKDVLTITLTPKVTCVVVDISK | |
| | DDPEVQFSWFVDDVEVHTAQTQPREEQENSTERSVSELPIMHQDW | |
| | LNGKEFKCRVNSAAFPAPIEKTISKTKGRPKAPQVYTIPPPKEQM | |
| | AKDKVSLTCMITDFFPEDITVEWQWNGQPAENYKNTQPIMDTDGS | |
| | YFVYSKLNVQKSNWEAGNTFTCSVLHEGLHNHHTEKSLSHSPGI | |
| SEQ ID NO: 73 | MFVFLVLLPLVSSQHHHHHHHHGGGSENLYFQSVASQSIIAYTMS | PxEBNCP11-S2 |
| | LGAENSVAYSNNSIAIPTNFTISVTTEILPVSMTKTSVDCTMYIC | |
| | GDSTECSNLLLQYGSFCTQLNRALTGIAVEQDKNTQEVFAQVKQI | |
| | YKTPPIKDFGGFNFSQILPDPSKPSKRSFIEDLLENKVTLADAGF | |
| | IKQYGDCLGDIAARDLICAQKENGLTVLPPLLTDEMIAQYTSALL | |
| | AGTITSGWTFGAGAALQIPFAMQMAYRENGIGVTQNVLYENQKLI | |
| | ANQFNSAIGKIQDSLSSTASALGKLQDVVNQNAQALNTLVKQLSS | |
| | NEGAISSVLNDILSRLDPPEAEVQIDRLITGRLQSLQTYVTQQLI | |
| | RAAEIRASANLAATKMSECVLGQSKRVDFCGKGYHLMSFPQSAPH | |
| | GVVFLHVTYVPAQEKNFTTAPAICHDGKAHFPREGVFVSNGTHWE | |
| | VTQRNFYEPQIITTDNTFVSGNCDVVIGIVNNTVYDPLQPELDSE | |
| | KEELDKYFKNHTSPDVDLGDISGINASVVNIQKEIDRLNEVAKNL | |
| | NESLIDLQELGKYEQYIKW | |
| SEQ ID NO: 74 | MFVFLVLLPLVSSQSVASQSIIAYTMSLGAENSVAYSNNSIAIPT | PxEBNCP13-S2 |
| | NFTISVTTEILPVSMTKTSVDCTMYICGDSTECSNLLLQYGSFCT | |
| | QLNRALTGIAVEQDKNTQEVFAQVKQIYKTPPIKDEGGENESQIL | |
| | PDPSKPSKRSFIEDLLENKVTLADAGFIKQYGDCLGDIAARDLIC | |
| | AQKFNGLTVLPPLLTDEMIAQYTSALLAGTITSGWTFGAGAALQI | |
| | PFAMQMAYRENGIGVTQNVLYENQKLIANQFNSAIGKIQDSLSST | |
| | ASALGKLQDVVNQNAQALNTLVKQLSSNFGAISSVLNDILSRLDP | |
| | PEAEVQIDRLITGRLQSLQTYVTQQLIRAAEIRASANLAATKMSE | |
| | CVLGQSKRVDFCGKGYHLMSFPQSAPHGVVFLHVTYVPAQEKNET | |
| | TAPAICHDGKAHFPREGVFVSNGTHWFVTQRNFYEPQIITTDNTF | |
| | VSGNCDVVIGIVNNTVYDPLQPELDSFKEELDKYFKNHTSPDVDL | |
| | GDISGINASVVNIQKEIDRLNEVAKNLNESLIDLQELGKYEQYIK | |
| | WPGGGSHHHHHHHH | |
| SEQ ID NO: 75 | MSSSSWLLLSLVAVTAAQSTIEEQAKTFLDKENHEAEDLFYQSSL | PxWP-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DNPQECLLLEPGLNEIMANSLDYNERLWAWESWRSEVGKQLRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPELLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLENMLRLGKSEPWTLALENVVGAKNMNVRPLL | |
| | NYFEPLFTWLKDQNKNSFVGWSTDWSPYADLEVLFQGPHHHHHH | |
| SEQ ID NO: 76 | MSSSSWLLLSLVAVTAAQHHHHHHHHGGGSENLYFQSTIEEQAKT | PxEBN2-ACE2 |
| | FLDKENHEAEDLFYQSSLASWNYNTNITEENVQNMNNAGDKWSAF | |
| | LKEQSTLAQMYPLQEIQNLTVKLQLQALQQNGSSVLSEDKSKRLN | |
| | TILNTMSTIYSTGKVCNPDAPAECLLLEPGLNEIMANSLDYNERL | |
| | WAWESWRSEVGKALRPLYEEYVVLKNEMARANHYEDYGDYWRGDY | |
| | EVNGVDGYDYSRGQLIEDVEHTFEEIKPLYEHLHAYVRAKLMNAY | |
| | PSYISPIGCLPAHLLGDMWGRFWTNLYSLTVPFGQKPNIDVTDAM | |
| | VDQAWDAQRIFKEAEKFFVSVGLPNMTQGFWENSMLTDPGNVQKA | |
| | VCHPTAWDLGKGDFRILMCTKVTMDDELTAHHEMGHIQYDMAYAA | |
| | QPFLLRNGANEGFHEAVGEIMSLSAATPKHLKSIGLLSPDFQEDN | |
| | ETEINFLLKQALTIVGTLPFTYMLEKWRWMVFKGEIPKDQWMKKW | |
| | WEMKREIVGVVEPVPHDETYCDPASLFHVSNDYSFIRYYTRTLYQ | |
| | FQFQEALCQAAKHEGPLHKCDISNSTEAGQKLENMLRLGKSEPWT | |
| | LALENVVGAKNMNVRPLLNYFEPLFTWLKDQNKNSFVGWSTDWSP | |
| | YAD | |
| SEQ ID NO: 77 | MSSSSWLLLSLVAVTAAQSTIEEQAKTFLDKENHEAEDLFYQSSL | PxEBN4-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPFLLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLENMLRLGKSEPWTLALENVVGAKNMNVRPLL | |
| | NYFEPLFTWLKDQNKNSFVGWSTDWSPYADGGGSHHHHHHHH | |
| SEQ ID NO: 78 | MSSSSWLLLSLVAVTAAQSTIEEQAKTELDKENHEAEDLFYQSSL | PxEBN6-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGREWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPELLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLENMLRLGKSEPWTLALENVVGAKNMNVRPLL | |
| | NYFEPLFTWLKDQNKNSFVGWSTDWSPYADLVPRGSASTKGPSVE | |
| | PLAPSSKSTSGGTAALGCLVKDYFPEPVTVSWNSGALTSGVHTFP | |
| | AVLQSSGLYSLSSVVTVPSSSLGTQTYICNVNHKPSNTKVDKKVE | |
| | PKSCDKTHTCPPCPAPELLGGPSVFLFPPKPKDTLMISRTPEVTC | |
| | VVVDVSHEDPEVKENWYVDGVEVHNAKTKPREEQYNSTYRVVSVL | |
| | TVLHQDWLNGKEYKCKVSNKALPAPIEKTISKAKGQPREPQVYTL | |
| | PPSRDELTKNQVSLTCLVKGFYPSDIAVEWESNGQPENNYKTTPP | |
| | VLDSDGSFFLYSKLTVDKSRWQQGNVESCSVMHEALHNHYTQKSL | |
| | SLSPGK | |
| SEQ ID NO: 79 | MSSSSWLLLSLVAVTAAQSTIEEQAKTFLDKENHEAEDLFYQSSL | PxEBN9-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPFLLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLFNMLRLGKSEPWTLALENVVGAKNMNVRPLL | |
| | NYFEPLFTWLKDQNKNSFVGWSTDWSPYADGGGSMDEKTTGWRGG | |
| | HVVEGLAGELEQLRARLEHHPQGQREPHHHHHHHH | |
| SEQ ID NO: 80 | MYRMQLLSCIALSLALQSTIEEQAKTFLDKENHEAEDLFYQSSLA | PxEBNCP9-ACE2 |
| | SWNYNTNITEENVQNMNNAGDKWSAFLKEQSTLAQMYPLQEIQNL | |
| | TVKLQLQALQQNGSSVLSEDKSKRLNTILNTMSTIYSTGKVCNPD | |
| | APAECLLLEPGLNEIMANSLDYNERLWAWESWRSEVGKALRPLYE | |
| | EYVVLKNEMARANHYEDYGDYWRGDYEVNGVDGYDYSRGQLIEDV | |
| | EHTFEEIKPLYEHLHAYVRAKLMNAYPSYISPIGCLPAHLLGDMW | |
| | GRFWTNLYSLTVPFGQKPNIDVTDAMVDQAWDAQRIFKEAEKFFV | |
| | SVGLPNMTQGFWENSMLTDPGNVQKAVCHPTAWDLGKGDFRILMC | |
| | TKVTMDDELTAHHEMGHIQYDMAYAAQPELLRNGANEGFHEAVGE | |
| | IMSLSAATPKHLKSIGLLSPDFQEDNETEINFLLKQALTIVGTLP | |
| | FTYMLEKWRWMVFKGEIPKDQWMKKWWEMKREIVGVVEPVPHDET | |
| | YCDPASLFHVSNDYSFIRYYTRTLYQFQFQEALCQAAKHEGPLHK | |
| | CDISNSTEAGQKLENMLRLGKSEPWTLALENVVGAKNMNVRPLLN | |
| | YFEPLFTWLKDQNKNSFVGWSTDWSPYAD | |
| SEQ ID NO: 81 | MDAMKRGLCCVLLLCGAVFVSPSQSTIEEQAKTFLDKENHEAEDL | PxEBNCP10-ACE2 |
| | FYQSSLASWNYNTNITEENVQNMNNAGDKWSAFLKEQSTLAQMYP | |
| | LQEIQNLTVKLQLQALQQNGSSVLSEDKSKRLNTILNTMSTIYST | |
| | GKVCNPDAPAECLLLEPGLNEIMANSLDYNERLWAWESWRSEVGK | |
| | ALRPLYEEYVVLKNEMARANHYEDYGDYWRGDYEVNGVDGYDYSR | |
| | GQLIEDVEHTFEEIKPLYEHLHAYVRAKLMNAYPSYISPIGCLPA | |
| | HLLGDMWGRFWTNLYSLTVPFGQKPNIDVTDAMVDQAWDAQRIFK | |
| | EAEKFFVSVGLPNMTQGFWENSMLTDPGNVQKAVCHPTAWDLGKG | |
| | DERILMCTKVTMDDELTAHHEMGHIQYDMAYAAQPELLRNGANEG | |
| | FHEAVGEIMSLSAATPKHLKSIGLLSPDFQEDNETEINFLLKQAL | |
| | TIVGTLPFTYMLEKWRWMVFKGEIPKDQWMKKWWEMKREIVGVVE | |
| | PVPHDETYCDPASLFHVSNDYSFIRYYTRTLYQFQFQEALCQAAK | |
| | HEGPLHKCDISNSTEAGQKLENMLRLGKSEPWTLALENVVGAKNM | |
| | NVRPLLNYFEPLFTWLKDQNKNSFVGWSTDWSPYAD | |
| SEQ ID NO: 82 | MSSSSWLLLSLVAVTAAQSTIEEQAKTELDKENHEAEDLFYQSSL | PxEBNCP13-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPFLLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLENMLRLGKSEPWTLALENVVGAKNMNVRPLL | |
| | NYFEPLFTWLKDQNKNSFVGWSTDWSPYADGGSGMDEKTTGWRGG | |
| | HVVEGLAGELEQLRARLEHHPQGQREP | |
| SEQ ID NO: 83 | MSSSSWLLLSLVAVTAAQSTIEEQAKTFLDKENHEAEDLFYQSSL | PxEBNCP14-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPELLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLENMLRLGKSEPWTLALENVVGAKNMNVRPLL | |
| | NYFEPLFTWLKDQNKNSFVGWSTDWSPYADGGSGWSHPQFEKGGG | |
| | SGGGSGGSAWSHPQFEK | |
| SEQ ID NO: 84 | MSSSSWLLLSLVAVTAAQSTIEEQAKTFLDKENHEAEDLFYQSSL | PxEBNCP15-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPELLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLENMLRLGKSEPWTLALENVVGAKNMNVRPLL | |
| | NYFEPLFTWLKDQNKNSFVGWSTDWSPYADGGSGWSHPQFEK | |
| SEQ ID NO: 85 | MSSSSWLLLSLVAVTAAQSTIEEQAKTFLDKENHEAEDLFYQSSL | PxEBNCP18-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGREWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPFLLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLENMLRLGKSEPWTLALENVVGAKNMNVRPLL | |
| | NYFEPLFTWLKDQNKNSFVGWSTDWSPYADGGGSKPLDGEYFTLQ | |
| | IRGRERFEMFRELNEALELKDAQAGKEPG | |
| SEQ ID NO: 86 | MEWSWVFLFFLSVTTGVHSQSTIEEQAKTFLDKENHEAEDLFYQS | PxEBNCP19-ACE2 |
| | SLASWNYNTNITEENVQNMNNAGDKWSAFLKEQSTLAQMYPLQEI | |
| | QNLTVKLQLQALQQNGSSVLSEDKSKRLNTILNTMSTIYSTGKVC | |
| | NPDAPAECLLLEPGLNEIMANSLDYNERLWAWESWRSEVGKALRP | |
| | LYEEYVVLKNEMARANHYEDYGDYWRGDYEVNGVDGYDYSRGQLI | |
| | EDVEHTFEEIKPLYEHLHAYVRAKLMNAYPSYISPIGCLPAHLLG | |
| | DMWGRFWTNLYSLTVPFGQKPNIDVTDAMVDQAWDAQRIFKEAEK | |
| | FFVSVGLPNMTQGFWENSMLTDPGNVQKAVCHPTAWDLGKGDERI | |
| | LMCTKVTMDDELTAHHEMGHIQYDMAYAAQPFLLRNGANEGFHEA | |
| | VGEIMSLSAATPKHLKSIGLLSPDFQEDNETEINFLLKQALTIVG | |
| | TLPFTYMLEKWRWMVFKGEIPKDQWMKKWWEMKREIVGVVEPVPH | |
| | DETYCDPASLFHVSNDYSFIRYYTRTLYQFQFQEALCQAAKHEGP | |
| | LHKCDISNSTEAGQKLFNMLRLGKSEPWTLALENVVGAKNMNVRP | |
| | LLNYFEPLFTWLKDQNKNSFVGWSTDWSPYAD | |
| SEQ ID NO: 87 | MSSSSWLLLSLVAVTAAQSTIEEQAKTELDKENHEAEDLFYQSSL | PxEBNCP20-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPELLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLFNMLRLGKSEPWTLALENVVGAKNMNVRPLL | |
| | NYFEPLFTWLKDQNKNSFVGWSTDWSPYADVPRDCGCKPCICTVP | |
| | EVSSVFIFPPKPKDVLTITLTPKVTCVVVDISKDDPEVQFSWFVD | |
| | DVEVHTAQTQPREEQFNSTERSVSELPIMHQDWLNGKEFKCRVNS | |
| | AAFPAPIEKTISKTKGRPKAPQVYTIPPPKEQMAKDKVSLTCMIT | |
| | DFFPEDITVEWQWNGQPAENYKNTQPIMNINGSYFVYSKLNVQKS | |
| | NWEAGNTFTCSVLHEGLHNHHTEKSLSHSPGKGGSGMDEKTTGWR | |
| | GGHVVEGLAGELEQLRARLEHHPQGQREP | |
| SEQ ID NO: 88 | MSSSSWLLLSLVAVTAAQSTIEEQAKTFLDKFNHEAEDLFYQSSL | PxEBNCP21-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPELLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLENMLRLGKSEPWTLALENVVGAKNMNVRPLL | |
| | NYFEPLFTWLKDQNKNSFVGWSTDWSPYADVPRDCGCKPCICTVP | |
| | EVSSVFIFPPKPKDVLTITLTPKVTCVVVDISKDDPEVQFSWFVD | |
| | DVEVHTAQTQPREEQFNSTERSVSELPIMHQDWLNGKEFKCRVNS | |
| | AAFPAPIEKTISKTKGRPKAPQVYTIPPPKEQMAKDKVSLTCMIT | |
| | DFFPEDITVEWQWNGQPAENYKNTQPIMNINGSYFVYSKLNVQKS | |
| | NWEAGNTFTCSVLHEGLHNHHTEKSLSHSPGKGGSGWSHPQFEKG | |
| | GGSGGGSGGSAWSHPQFEK | |
| SEQ ID NO: 89 | MSSSSWLLLSLVAVTAAQSTIEEQAKTFLDKENHEAEDLFYQSSL | PxEBNCP22-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPELLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLENMLRLGKSEPWTLALENVVGAKNMNVRPLL | |
| | NYFEPLFTWLKDQNKNSFVGWSTDWSPYADVPRDCGCKPCICTVP | |
| | EVSSVFIFPPKPKDVLTITLTPKVTCVVVDISKDDPEVQFSWEVD | |
| | DVEVHTAQTQPREEQFNSTERSVSELPIMHQDWLNGKEFKCRVNS | |
| | AAFPAPIEKTISKTKGRPKAPQVYTIPPPKEQMAKDKVSLTCMIT | |
| | DFFPEDITVEWQWNGQPAENYKNTQPIMNINGSYFVYSKLNVQKS | |
| | NWEAGNTFTCSVLHEGLHNHHTEKSLSHSPGK | |
| SEQ ID NO: 90 | MSSSSWLLLSLVAVTAAQSTIEEQAKTFLDKENHEAEDLFYQSSL | PxEBNCP23-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPELLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLENMLRLGKSEPWTLALENVVGAKNMNVRPLL | |
| | NYFEPLFTWLKDQNKNSFVGWSTDWSPYADVPEVSSVFIFPPKPK | |
| | DVLTITLTPKVTCVVVDISKDDPEVQFSWFVDDVEVHTAQTQPRE | |
| | EQFNSTFRSVSELPIMHQDWLNGKEFKCRVNSAAFPAPIEKTISK | |
| | TKGRPKAPQVYTIPPPKEQMAKDKVSLTCMITDFFPEDITVEWQW | |
| | NGQPAENYKNTQPIMNINGSYFVYSKLNVQKSNWEAGNTFTCSVL | |
| | HEGLHNHHTEKSLSHSPGK | |
| SEQ ID NO: 91 | MSSSSWLLLSLVAVTAAQSTIEEQAKTFLDKENHEAEDLFYQSSL | PxEBNCP24-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPELLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLENMLRLGKSEPWTLALENVVGAKNMNVRPLL | |
| | NYFEPLFTWLKDQNKNSFVGWSTDWSPYADGGGSKPLDGEYFTLQ | |
| | IRGRERFEMFRELNEALELKDAQAGKEPGGGSGMDEKTTGWRGGH | |
| | VVEGLAGELEQLRARLEHHPQGQREP | |
| SEQ ID NO: 92 | MSSSSWLLLSLVAVTAAQSTIEEQAKTELDKENHEAEDLFYQSSL | PxEBNCP25-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPELLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYSFIRYYTRTLYQFQFQEALCQAAKHEGPLH | |
| | KCDISNSTEAGQKLENMLRLGKSEPWTLALENVVGAKNMNVRPLL | |
| | NYFEPLFTWLKDQNKNSFVGWSTDWSPYAD | |
| SEQ ID NO: 93 | MSSSSWLLLSLVAVTAAQSTIEEQAKTFLDKENHEAEDLFYQSSL | PxEBNCP26-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDFRILM | |
| | CTKVTMDDELTAHHEMGHIQYDMAYAAQPFLLRNGANEGFHEAVG | |
| | EIMSLSAATPKHLKSIG | |
| SEQ ID NO: 94 | MSSSSWLLLSLVAVTAAQSTIEEQAKTELDKENHEAEDLFYQSSL | PxEBNCP27-ACE2 |
| | ASWNYNTNITEENVQNMNNAGDKWSAFLKEQSTLAQMYPLQEIQN | |
| | LTVKLQLQALQQNGSSVLSEDKSKRLNTILNTMSTIYSTGKVCNP | |
| | DAPAECLLLEPGLNEIMANSLDYNERLWAWESWRSEVGKALRPLY | |
| | EEYVVLKNEMARANHYEDYGDYWRGDYEVNGVDGYDYSRGQLIED | |
| | VEHTFEEIKPLYEHLHAYVRAKLMNAYPSYISPIGCLPAHLLGDM | |
| | WGRFWTNLYSLTVPFGQKPNIDVTDAMVDQAWDAQRIFKEAEKFF | |
| | VSVGLPNMTQGFWENSMLTDPGNVQKAVCHPTAWDLGKGDERILM | |
| | CTKVTMDDELTAHHEMGHIQYDMAYAAQPELLRNGANEGFHEAVG | |
| | EIMSLSAATPKHLKSIGLLSPDFQEDNETEINFLLKQALTIVGTL | |
| | PFTYMLEKWRWMVFKGEIPKDQWMKKWWEMKREIVGVVEPVPHDE | |
| | TYCDPASLFHVSNDYS | |
| SEQ ID NO: 95 | MFVFLVLLPLVSSQCVNLTTRTQLPPAYTNSFTRGVYYPDKVERS | pxEBNCP21-FLS |
| | SVLHSTQDLFLPFFSNVTWFHAIHVSGTNGTKRFDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDETGCVIAWNSNNLDSKVGGNYN | |
| | YLYRLERKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGENFSQILPDPS | |
| | KPSKRSFIEDLLENKVTLADAGFIKQYGDCLGDIAARDLICAQKF | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSWKQSVELAKKDSLYKDAMQYASESKDTELAEELLQWFLQEEKR | |
| | ECFGACLFTCYDLLRPDVVLELAWRHNIMDEAMPYFIQVMKEYLT | |
| | KVDKLDASESLRKEEEGGGSHHHHHHHH | |
| SEQ ID NO: 96 | MFVFLVLLPLVSSQCVNLTTRTQLPPAYTNSFTRGVYYPDKVERS | pxENBCP22-FLS |
| | SVLHSTQDLFLPFFSNVTWFHAIHVSGTNGTKREDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDETGCVIAWNSNNLDSKVGGNYN | |
| | YLYRLERKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGENFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSRMKQLEDKVEELLSKNYHLENEVARLKKLVGERGGGSHHHHHH | |
| | HH | |
| SEQ ID NO: 97 | MFVFLVLLPLVSSQCVNLTTRTQLPPAYTNSFTRGVYYPDKVERS | pxENBCP23-FLS |
| | SVLHSTQDLFLPFFSNVTWFHAIHVSGTNGTKRFDNPVLPENDGV | |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YLYRLFRKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQDVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGENFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSASSGVRLWATRQAMLGQVHEVPEGWLIFVAEQEELYVRVQNGE | |
| | RKVQLEARTPLPRGGGSHHHHHHHH | |
| SEQ ID NO: 98 | MFVFLVLLPLVSSQCVNLTTRTQLPPAYTNSFTRGVYYPDKVERS | pxENB9-FLS-B1.351 |
| | SVLHSTQDLFLPFFSNVTWFHAISGTNGTKRFDNPVLPENDGVYF | |
| | ASTEKSNIIRGWIFGTTLDSKTQSLLIVNNATNVVIKVCEFQFCN | |
| | DPFLGVYHKNNKSWMESEFRVYSSANNCTFEYVSQPFLMDLEGKQ | |
| | GNFKNLREFVEKNIDGYFKIYSKHTPINLVRDLPQGFSALEPLVD | |
| | LPIGINITRFQTLLALHRSYLTPGDSSSGWTAGAAAYYVGYLQPR | |
| | TELLKYNENGTITDAVDCALDPLSETKCTLKSFTVEKGIYQTSNE | |
| | RVQPTESIVRFPNITNLCPFGEVENATRFASVYAWNRKRISNCVA | |
| | DYSVLYNSASFSTFKCYGVSPTKLNDLCFTNVYADSFVIRGDEVR | |
| | QIAPGQTGKIADYNYKLPDDFTGCVIAWNSNNLDSKVGGNYNYLY | |
| | RLFRKSNLKPFERDISTEIYQAGSTPCNGVEGENCYFPLQSYGFQ | |
| | PTYGVGYQPYRVVVLSFELLHAPATVCGPKKSTNLVKNKCVNENE | |
| | NGLTGTGVLTESNKKELPFQQFGRDIDDTTDAVRDPQTLEILDIT | |
| | PCSFGGVSVITPGTNTSNQVAVLYQGVNCTEVPVAIHADQLTPTW | |
| | RVYSTGSNVFQTRAGCLIGAEHVNNSYECDIPIGAGICASYQTQT | |
| | NSHGAASSVASQSIIAYTMSLGAENSVAYSNNSIAIPINFTISVT | |
| | TEILPVSMTKTSVDCTMYICGDSTECSNLLLQYGSFCTQLNRALT | |
| | GIAVEQDKNTQEVFAQVKQIYKTPPIKDFGGFNFSQILPDPSKPS | |
| | KRSFIEDLLENKVTLADAGFIKQYGDCLGDIAARDLICAQKENGL | |
| | TVLPPLLTDEMIAQYTSALLAGTITSGWTFGAGAALQIPFAMQMA | |
| | YRENGIGVTQNVLYENQKLIANQFNSAIGKIQDSLSSTASALGKL | |
| | QDVVNQNAQALNTLVKQLSSNFGAISSVLNDILARLDPPEAEVQI | |
| | DRLITGRLQSLQTYVTQQLIRAAEIRASANLAATKMSECVLGQSK | |
| | RVDFCGKGYHLMSFPQSAPHGVVFLHVTYVPAQEKNFTTAPAICH | |
| | DGKAHFPREGVFVSNGTHWFVTQRNFYEPQIITTHNTFVSGNCDV | |
| | VIGIVNNTVYDPLQPELDSFKEELDKYFKNHTSPDVDLGDISGIN | |
| | ASVVNIQKEIDRLNEVAKNLNESLIDLQELGKYEQYIKWPGGGSG | |
| | YIPEAPRDGQAYVRKDGEWVLLSTFLGGGSHHHHHHHH | |
| SEQ ID NO: 99 | MFVFLVLLPLVSSQCVNLRTRTQLPPAYTNSFTRGVYYPDKVERS | pxENB9-FLS- |
| | SVLHSTQDLFLPFFSNVTWFHAIHVSGTNGTKRFDNPVLPENDGV | B1.617.2 |
| | YFASTEKSNIIRGWIFGTTLDSKTQSLLIVNNATNVVIKVCEFQF | |
| | CNDPFLDVYYHKNNKSWMESGVYSSANNCTFEYVSQPFLMDLEGK | |
| | QGNFKNLREFVEKNIDGYFKIYSKHTPINLVRDLPQGFSALEPLV | |
| | DLPIGINITRFQTLLALHRSYLTPGDSSSGWTAGAAAYYVGYLQP | |
| | RTFLLKYNENGTITDAVDCALDPLSETKCTLKSFTVEKGIYQTSN | |
| | FRVQPTESIVRFPNITNLCPFGEVENATRFASVYAWNRKRISNCV | |
| | ADYSVLYNSASFSTFKCYGVSPTKLNDLCFTNVYADSFVIRGDEV | |
| | RQIAPGQTGKIADYNYKLPDDFTGCVIAWNSNNLDSKVGGNYNYR | |
| | YRLERKSNLKPFERDISTEIYQAGSKPCNGVEGENCYFPLQSYGE | |
| | QPTNGVGYQPYRVVVLSFELLHAPATVCGPKKSTNLVKNKCVNEN | |
| | FNGLTGTGVLTESNKKELPFQQFGRDIADTTDAVRDPQTLEILDI | |
| | TPCSFGGVSVITPGTNTSNQVAVLYQGVNCTEVPVAIHADQLTPT | |
| | WRVYSTGSNVFQTRAGCLIGAEHVNNSYECDIPIGAGICASYQTQ | |
| | TNSRGAASSVASQSIIAYTMSLGAENSVAYSNNSIAIPTNFTISV | |
| | TTEILPVSMTKTSVDCTMYICGDSTECSNLLLQYGSFCTQLNRAL | |
| | TGIAVEQDKNTQEVFAQVKQIYKTPPIKDFGGFNFSQILPDPSKP | |
| | SKRSFIEDLLENKVTLADAGFIKQYGDCLGDIAARDLICAQKENG | |
| | LTVLPPLLTDEMIAQYTSALLAGTITSGWTFGAGAALQIPFAMQM | |
| | AYRENGIGVTQNVLYENQKLIANQFNSAIGKIQDSLSSTASALGK | |
| | LQNVVNQNAQALNTLVKQLSSNFGAISSVLNDILSRLDPPEAEVQ | |
| | IDRLITGRLQSLQTYVTQQLIRAAEIRASANLAATKMSECVLGQS | |
| | KRVDFCGKGYHLMSFPQSAPHGVVFLHVTYVPAQEKNFTTAPAIC | |
| | HDGKAHFPREGVFVSNGTHWFVTQRNFYEPQIITTDNTFVSGNCD | |
| | VVIGIVNNTVYDPLQPELDSFKEELDKYFKNHTSPDVDLGDISGI | |
| | NASVVNIQKEIDRLNEVAKNLNESLIDLQELGKYEQYIKWPGGGS | |
| | GYIPEAPRDGQAYVRKDGEWVLLSTFLGGGSHHHHHHHH | |
| SEQ ID NO: | MFVFLVLLPLVSSQCVNLTTRTQLPPAYTNSFTRGVYYPDKVERS | pxENB9-FLS- |
| 100 | SVLHSTQDLFLPFFSNVTWFHAIHVSGTNGTKRFDNPVLPENDGV | B1.4.27 |
| | YFASTEKSNIIRGWIFGTTLDSKTQSLLIVNNATNVVIKVCEFQF | |
| | CNDPFLGVYYHKNNKSWMESEFRVYSSANNCTFEYVSQPFLMDLE | |
| | GKQGNFKNLREFVEKNIDGYFKIYSKHTPINLVRDLPQGESALEP | |
| | LVDLPIGINITRFQTLLALHRSYLTPGDSSSGWTAGAAAYYVGYL | |
| | QPRTELLKYNENGTITDAVDCALDPLSETKCTLKSFTVEKGIYQT | |
| | SNFRVQPTESIVRFPNITNLCPFGEVENATRFASVYAWNRKRISN | |
| | CVADYSVLYNSASFSTFKCYGVSPTKLNDLCFTNVYADSFVIRGD | |
| | EVRQIAPGQTGKIADYNYKLPDDFTGCVIAWNSNNLDSKVGGNYN | |
| | YRYRLFRKSNLKPFERDISTEIYQAGSTPCNGVEGENCYFPLQSY | |
| | GFQPTNGVGYQPYRVVVLSFELLHAPATVCGPKKSTNLVKNKCVN | |
| | FNFNGLTGTGVLTESNKKELPFQQFGRDIADTTDAVRDPQTLEIL | |
| | DITPCSFGGVSVITPGTNTSNQVAVLYQGVNCTEVPVAIHADQLT | |
| | PTWRVYSTGSNVFQTRAGCLIGAEHVNNSYECDIPIGAGICASYQ | |
| | TQTNSPGAASSVASQSIIAYTMSLGAENSVAYSNNSIAIPTNFTI | |
| | SVTTEILPVSMTKTSVDCTMYICGDSTECSNLLLQYGSFCTQLNR | |
| | ALTGIAVEQDKNTQEVFAQVKQIYKTPPIKDFGGENFSQILPDPS | |
| | KPSKRSFIEDLLENKVTLADAGFIKQYGDCLGDIAARDLICAQKE | |
| | NGLTVLPPLLTDEMIAQYTSALLAGTITSGWTFGAGAALQIPFAM | |
| | QMAYRENGIGVTQNVLYENQKLIANQFNSAIGKIQDSLSSTASAL | |
| | GKLQDVVNQNAQALNTLVKQLSSNFGAISSVLNDILSRLDPPEAE | |
| | VQIDRLITGRLQSLQTYVTQQLIRAAEIRASANLAATKMSECVLG | |
| | QSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGN | |
| | CDVVIGIVNNTVYDPLQPELDSFKEELDKYFKNHTSPDVDLGDIS | |
| | GINASVVNIQKEIDRLNEVAKNLNESLIDLQELGKYEQYIKWPGG | |
| | GSGYIPEAPRDGQAYVRKDGEWVLLSTFLGGGSHHHHHHHH | |

1. A fusion protein comprising:
at least one selected from the group consisting of a full length SARS-CoV-2 spike protein, the S1 domain or the S2 domain of the SARS-CoV-2 spike protein or a fragment thereof, and a human Angiotensin Converting Enzyme 2 (ACE2) receptor of the SARS-CoV-2 spike protein or a fragment thereof;
an N-terminal signal peptide; and
at least one selected from the group consisting of a polyhistidine tag, a streptavidin binding domain, a linker, and an oligomerization tag.

2. The fusion protein, according to claim 1, wherein said N-terminal signal peptide is selected from the group consisting of a spike endogenous signal peptide, a tissue plasminogen activator (tPa) signal peptide, a human interleukin 2 (hIL2) signal peptide, a murine immunoglobulin G (IgG) heavy chain signal peptide and a angiotensin-converting enzyme 2 (ACE2) signal peptide.

3. (canceled)

4. The fusion protein, according to claim 1, wherein said fusion protein comprises the linker, wherein said linker is a flexible linker or a kinked linker.

5. (canceled)

6. The fusion protein, according to claim 1, wherein said fusion protein comprises the polyhistidine tag, wherein the polyhistidine tag consists of 4, 6, 8, 10, or 12 histidine residues.

7. (canceled)

8. The fusion protein, according to claim 1, wherein said fusion protein comprises the oligomerization tag, wherein the oligomerization tag is selected from the group consisting of a T4 fibritin foldon domain, a short T4 fibritin foldon domain, a clathrin trimerization domain, a human collagen trimerization domain, a CGN4 trimerization domain, a tetranectin trimerization domain, a p53 tetramerization domain and a murine or human Fc domain.

9. (canceled)

10. The fusion protein, according to claim 1, wherein the fusion protein comprises the streptavidin binding domain, wherein the streptavidin binding domain has an amino acid sequence selected from the group consisting of SEQ ID NO: 29, SEQ ID NO: 30, or SEQ ID NO: 31.

11. The fusion protein, according to claim 1, wherein the fusion protein comprises said full length SARS-CoV-2 spike protein, wherein the full length SARS-CoV-2 spike protein has the amino acid sequence set forth in SEQ ID NO: 32: wherein the fusion protein comprises said S1 domain of the SARS-CoV-2 spike protein or a fragment thereof, wherein the S1 domain of the SARS-CoV-2 spike protein or a fragment thereof has the amino acid sequence set forth in SEQ ID NO: 33; wherein the fusion protein comprises said S2 domain of the SARS-CoV-2 spike protein or a fragment thereof, wherein the S2 domain of the SARS-CoV-2 spike protein or a fragment thereof has the amino acid sequence set forth in SEQ ID NO: 35.

12. (canceled)

13. (canceled)

14. The fusion protein, according to claim 1, wherein the fusion protein comprises said human Angiotensin Converting Enzyme 2 (ACE2) receptor of the SARS-CoV-2 spike protein or a fragment thereof, wherein the ACE2 receptor of the SARS-CoV-2 spike protein or a fragment thereof has the amino acid sequence set forth in SEQ ID NO: 38.

15. The fusion protein, according to claim 1, wherein the full length SARS-CoV-2 spike protein, or the S1 domain or the S2 domain of the SARS-CoV-2 spike protein or a fragment thereof, comprises a mutation in one or more of the following positions: N354, D364, V367, G404, R408, A435, W436, G467, A475, T478, N481, G485, F490, Q493, G496, Q498, N501, G502, V503 or D614, relative to SEQ ID NO: 32.

16. The fusion protein, according to claim 1, wherein the fusion protein comprises said ACE2 protein or the fragment thereof, wherein the ACE2 protein or the fragment thereof comprises three alanine mutations which are Q139A, Q175A and N137A, relative to SEQ ID NO: 38.

17. The fusion protein, according to claim 1, wherein said fusion protein comprises the stabilizing mutations K986P and V987P, relative to SEQ ID NO: 32.

18. (canceled)

19. The fusion protein, according to claim 1, wherein said fusion protein contains a Thrombin cleavage site, a TEV cleavage site or a HRV 3C Protease Cleavage Site.

20. The fusion protein, according to claim 1, wherein said fusion protein comprise the amino acid sequence selected from the group consisting of SEQ ID NO: 37, and SEQ ID NO: 40 to SEQ ID NO: 100.

21. The fusion protein, according to claim 20, wherein the fusion protein comprises the full length the SARS-CoV-2 spike protein, and comprises the amino acid sequence selected from the group consisting of SEQ ID NO: 37, SEQ ID NO: 40 to SEQ ID NO: 62 and SEQ ID NO: 95 to SEQ ID NO: 100; wherein the fusion protein comprises the S1 domain of the SARS-CoV-2 spike protein or the fragment thereof, and comprises the amino acid sequence selected from the group consisting of SEQ ID NO: 63 to 72; wherein the fusion protein comprises the S2 domain of the SARS-CoV-2 spike protein of the fragment thereof, and comprises the amino acid sequence selected from the group consisting of SEQ ID NO: 73 to 74.

22. (canceled)

23. (canceled)

24. The fusion protein, according to claim 20, wherein said fusion protein comprises the human Angiotensin Converting Enzyme 2 (ACE2) receptor of the SARS-CoV-2 spike protein or the fragment thereof, and comprises the amino acid sequence selected from the group consisting of SEQ ID NO: 75 to 94.

25. A cell, comprising the fusion protein according to claim 1.

26. A nucleic acid comprising a nucleotide sequence encoding the fusion protein according to claim 1, a promoter operably linked to the nucleotide sequence and a selectable marker.

27. A cell comprising the nucleic acid of claim 26.

28. A composition comprising the fusion protein of claim 1, and a solid support, wherein the fusion protein is covalently or non-covalently bound to the solid support.
